(19) United States
(12) Patent Application Publication
(10) Pub. No.: US 2023/0118142 A1
(43) Pub. Date: Apr. 20, 2023

(54) AGENTS FOR USE IN THE TREATMENT OF TISSUE DAMAGE

(71) Applicant: UCL BUSINESS LTD, London (GB)

(72) Inventors: Mark Brian PEPYS, London (GB); Christopher SWAIN, London (GB); Graham Walter TAYLOR, London (GB); Stephen Paul WOOD, London (GB); Melanie Susanne GLOSSOP, Stevenage (GB); Charlotte Alice Louise LANE, Stevenage (GB)

(21) Appl. No.: 17/904,388

(22) PCT Filed: Feb. 18, 2021
(86) PCT No.: PCT/EP2021/054071

§ 371 Date: Aug. 17, 2022

(30) Foreign Application Priority Data
Feb. 19, 2020 (GB) 2002299.2

(51) Int. Cl. A61K 31/439; A61K 31/662; A61K 31/4025

(52) U.S. Cl. CPC A61K 31/439; A61K 31/662; A61K 31/4025

(57) ABSTRACT

The invention comprises an agent for use in medicine, wherein the agent comprises a compound of Formula (I): B-L-B′ wherein: B and B′ are independently selected from groups of formula (B-I) wherein: Z is selected from —COOH, —CH₂COOH, —PO(OH)(OR¹), or —CH₂PO(OH) (OR¹), wherein R¹ is H or a phosphate protecting group; W is an alicyclic amine group having from 5 to 12 carbon atoms and at least one amine nitrogen atom; W′ is H, or W′ is linked to W to form said alicyclic amine group; and Y is selected from —NH—, —N(CH₃)—, —CH₂—, —NHCO—, —CH₂CONH—, —CONH—, CH₂NHCO—, or —NHCH₂—; and L is a linker group.

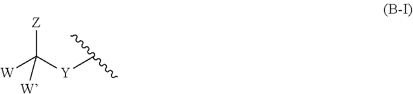

### FIELD OF THE INVENTION

The present invention relates to agents that are specifically bound by C-reactive protein (CRP) in vivo, thereby inhibiting the binding of CRP to autologous cellular and tissue ligands, and to compositions containing such agents for use in the treatment or prevention of tissue damage, in particular in ischaemic, traumatic, infectious, inflammatory and neoplastic conditions.

### BACKGROUND OF THE INVENTION

C-reactive protein (CRP) is a normal plasma protein of the pentraxin protein family, the other member of which is serum amyloid P component (SAP) (1) CRP is the classical acute phase protein, the circulating concentration of which increases dramatically in response to most forms of tissue injury, infection, inflammation and cancer. In most conditions the CRP value attained correlates closely with the extent and activity of disease. CRP is a calcium dependent ligand binding protein, which binds with highest affinity to phosphocholine residues, though it also binds a variety of other ligands of both autologous and extrinsic origin. Autologous ligands include native and modified plasma lipoproteins, damaged cell membranes, a number of different phospholipids and related compounds, and small nuclear ribonucleoprotein particles. Extrinsic ligands include some glycan, phospholipid and other components of micro-organisms, such as capsular and somatic components of bacteria, fungi and parasites, as well as plant products. CRP bound to macromolecular ligands activates the classical complement pathway via C1q, leading to activation and fixation of C3, the main adhesion molecule of the complement system, production of the major chemotactic factors, C3a and C5a, and engagement of the terminal lytic phase, C5-C9.

In addition to closely reflecting the extent and activity of whatever disease process has triggered increased CRP production, higher circulating concentrations of CRP also significantly predict progression of disease, incidence of complications and clinical outcome. Extensive clinical observations of this association, across a wide spectrum of diseases, are consistent with a pathogenic role of CRP in exacerbating tissue damage and thus disease severity. CRP does not bind to normal healthy cells but binds avidly to ligands exposed on dead and damaged cells and it then activates complement. Whilst CRP-mediated complement activation may contribute to clearance of cellular debris from the tissues and to host defence against some micro-organisms, it is clear that, just as in many antibody-mediated hypersensitivity reactions, complement activation can cause severe tissue damage.

The complement dependent pathogenicity of human CRP was first confirmed experimentally by the demonstration that administration of human CRP to rats undergoing coronary artery ligation increased the size of the resulting acute myocardial infarcts (2). Human CRP and activated rat complement were deposited in and around the infarct and the exacerbation of tissue damage was absolutely complement dependent. Similar observations were made in the middle cerebral artery occlusion model of stroke in rats (3). Subsequently several different independent groups have made comparable observations in a range of different animal models.

The design of the first small molecule inhibitor of CRP binding for use in vivo, bis(phosphocholine)hexane (BPC6), enabled conclusive confirmation of the pathogenic role of human CRP in exacerbating tissue damage after ischaemic infarction (4). Administration of this compound to rats undergoing coronary artery ligation and receiving human CRP completely abrogated the increased damage that occurred in human CRP treated animals which did not receive the treatment. Subsequently, bis(phosphocholine)octane (BPC8), was found to be a more potent inhibitor of CRP binding in vitro and it had the same protective effect against human CRP pathogenicity in the rat acute myocardial infarction model, including the ischaemia reperfusion design as well as after terminal coronary artery ligation (Pepys, unpublished observations). Human CRP was thus validated as a therapeutic target and efficacy of intervention via a small molecule inhibitor of CRP binding was demonstrated.

These observations opened the way to a novel avenue for reducing disease severity in the very wide variety of tissue damaging conditions in which there are increased circulating concentrations of CRP. Inhibition of CRP binding in vivo will obviously not prevent or cure diverse diseases with very different aetiologies. However, reducing the extent, severity and duration of tissue damage and thus prolonging survival in patients with heart attacks, strokes, rheumatoid arthritis and other chronic inflammatory disease of unknown cause, burns, bacterial and viral infections or cancer cachexia, and many other conditions, remains an urgent major unmet medical need.

WO03/097104 A1 describes an agent that is bound by CRP and inhibits CRP binding or other ligands. The agent comprises a plurality of ligands covalently co-linked so as to form a complex with a plurality of C-reactive protein (CRP) molecules, wherein (i) at least two of the ligands are the same or different and are capable of being bound by ligand binding sites present on the CRP molecules; or (ii) at least one of the ligands is capable of being bound by a ligand binding site present on a CRP molecule, and at least one other of the ligands is capable of being bound by a ligand binding site present on a serum amyloid P component (SAP) molecule. Suitable ligands for CRP are bis(phosphocholine) ligands, and an exemplified compound, designated BPC8, has the following formula (BPC8):

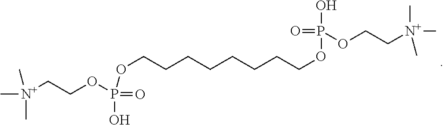

The number 8 in BPC8 refers to the n-octyl linker group in the above formula. Corresponding compounds BPC6, BPC7, etc. having n-hexyl, n-heptyl, etc. linker groups are also disclosed.

BPC6 and BPC8 are avidly bound by CRP, cross linking pairs of the native pentameric protein molecules. They completely abrogate the adverse effects of human CRP in the rat acute myocardial infarction model (4 and Pepys et al., unpublished observations). However, the bis(phosphocholine) alkane series of compounds were difficult to synthesise and purify at scale.

A need therefore remains for agents or compounds that are more easily prepared for use in the treatment of medical conditions which are exacerbated by CRP and which provide improved properties over the compounds described in the prior art.

### SUMMARY OF THE INVENTION

In a first aspect, there is provided an agent for use in medicine, wherein the agent comprises a compound of Formula (I):

B-L-B′

wherein:
B and B′ are independently selected from groups of formula:

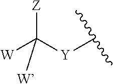

wherein:
Z is selected from —COOH, —CH₂COOH, —PO(OH)(OR¹), or —CH₂PO(OH)(OR¹),
wherein R¹ is a phosphate protecting group;
W is an alicyclic amine group having from 5 to 12 carbon atoms and at least one amine nitrogen atom;
W′ is H, or W′ is linked to W to form said alicyclic amine group; and
Y is selected from —NH—, —N(CH₃)—, —CH₂—, —NHCO—, —CH₂CONH—, —CONH—, —CH₂NHCO—, or —NHCH₂—; and
L is a linker group selected from: a direct bond; a saturated or unsaturated chain of from 1 to 12 carbon atoms in which from 1 to 4 of the carbon atoms are optionally replaced by O or S, and wherein the chain is optionally substituted by one or more groups selected from halogen, C1-C6 alkyl, C2-C6 alkenyl, C6-C12 (hetero)aryl, C6-C12 (hetero)arylC1-C4alkyl, or C1-C6 alkoxy; or L is a group of formula -L¹-Cy-L²- wherein Cy is a (hetero)aryl or (hetero)cycloalkyl group and L¹ and L² are independently selected from a direct bond or C1-C4 alkenyl groups in which one or two of the carbon atoms are optionally replaced by O or S, including stereoisomers (including enantiomers, diastereoisomers and racemic and scalemic mixtures thereof), and pharmaceutically acceptable salts, solvates, prodrugs or derivatives thereof.

Suitable and/or preferred agents according to the invention as defined above are described in detail below, and in the accompanying claims.

In another aspect, the present invention provides an agent for use in medicine comprising a compound of Formula (XII):

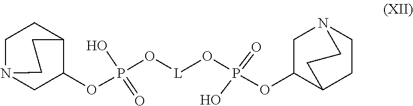

wherein L is a linker group as defined above in relation to the first aspect of the invention. Suitably, L is a linear or branched alkylene group of formula —CₙH₂ₙ— wherein n is from 1 to about 12, or a linear or branched alkenylene group of formula —CₙH₂ₙ₋₂— wherein n is from 1 to about 12. More suitably, L is a linear alkylene group of formula —(CH₂)ₙ— wherein n is from 1 to about 12, more suitably from 5 to 10, still more suitably wherein n is an even number, for example wherein n is 2, 4, 6 or 8.

The invention also encompasses any stereoisomer, enantiomer or geometric isomer of the agents disclosed herein, and mixtures thereof.

In another aspect, the present invention provides an agent according to the invention for use in the treatment or prevention of tissue damage in a subject having an inflammatory and/or tissue damaging condition.

In another aspect, the present invention provides the use of an agent according to the invention, for the manufacture of a medicament for treatment or prevention of tissue damage in a subject having an inflammatory and/or tissue damaging condition.

In another aspect, the present invention provides a pharmaceutical composition comprising an agent according to the invention in admixture with one or more pharmaceutically acceptable excipients, diluents or carriers.

The agents according to the invention, comprising the compound of Formula (I), may be administered concurrently with one or more other pharmaceutically active medications, simultaneously, separately or sequentially. Such other pharmaceutically active medications may include, for example, anti-inflammatory drugs such as corticosteroids; anti-viral, anti-bacterial, anti-fungal or anti-parasitic drugs; inhibitors/antagonists of pro-inflammatory cytokines such as IL-1, IL-6, TNF; anti-coagulants; inhibitors of complement activation or its bioactive fragments.

In a further aspect, the present invention provides a method for treatment or prevention of an inflammatory and/or tissue damaging condition in a patient in need thereof, comprising administering to the patient a therapeutic amount of an agent according to the first aspect of the invention or a pharmaceutical composition according to the invention.

The inflammatory and/or tissue damaging condition may comprise acute coronary syndrome/unstable angina/plaque rupture/incipient atherothrombosis. Or the inflammatory and/or tissue damaging condition is selected from an infection, an allergic complication of infection, an inflammatory disease, ischemic or other necrosis, traumatic tissue damage and malignant neoplasia.

In embodiments, the condition is an infection selected from a bacterial infection including sepsis, a mycobacterial infection, a viral infection, a fungal infection and a parasitic infection, and including complex tissue damaging conditions in which infection is a component, such as chronic obstructive pulmonary disease (COPD).

In embodiments, the condition is an inflammatory disease selected from rheumatoid arthritis, juvenile chronic (rheumatoid) arthritis, ankylosing spondylitis, psoriatic arthritis, systemic vasculitis, polymyalgia rheumatica, Reiter's disease, Crohn's disease and auto-inflammatory diseases.

In embodiments, the condition is tissue necrosis selected from myocardial infarction, ischaemic stroke, tumour embolization and acute pancreatitis.

In embodiments, the condition is trauma selected from elective surgery, burns, chemical injury, fractures and compression injury.

In embodiments, the condition is malignant neoplasia selected from lymphoma, Hodgkin's disease, carcinoma and sarcoma, and the terminal cachexia caused by any of these.

In embodiments, the condition is an allergic complication of infection selected from rheumatic fever, glomerulonephritis, and erythema nodosum leprosum.

In embodiments, the condition is an infection or complication of infection with a severe acute respiratory syndrome (SARS) coronavirus, in particular SARS-Cov-2.

Suitably, the method involves administering to a patient an amount of the agent according to the invention sufficient to be bound by all soluble CRP in the circulation and extracellular tissue fluids. For example, the amount may be sufficient to be bound by at least about 70% of the available CRP, preferably at least about 90% of available CRP and optimally 95%, 99% or 100% of the available CRP.

### DETAILED DESCRIPTION OF THE INVENTION

In the present patent application, including the accompanying claims, the aforementioned substituents have the following meanings:

Halogen atom or “halo” means fluorine, chlorine, bromine or iodine.

Alkyl groups and portions thereof (unless otherwise defined) maybe a straight or branched chain or cycloalkyl.

The term “C1-Cn alkyl” as used here refers to a straight or branched chain or cyclic carbon chain consisting of 1 to n carbon atoms, which can be optionally substituted by one or more halogens.

The term “C2-Cn alkenyl” as used here refers to a chain consisting of 2 to n carbon atoms, which contains one double bond which can be located in any position of the respective unsaturated radical.

The term “C2-Cn alkynyl” as used here refers to a chain consisting of 2 to n carbon atoms, which contains one triple bond which can be located in any position of the respective unsaturated moiety.

The term “C1-Cn alkoxy” as used here refers to a straight or branched or cyclic carbon chain consisting of 1 to n carbon atoms, which is connected via an oxygen atom to another group.

Pharmaceutically-acceptable salts of the agents disclosed herein include salts with a base or acid, which may be organic or inorganic. Salts of inorganic bases include those of alkali metals, alkaline earth metals and ammonium salts. Organic bases include pyridine, trimethylamine, triethylamine, ethanolamine, lysine, or the like. Inorganic acids include hydrochloric acid, sulphuric acid, nitric acid and phosphoric acid. Organic acids include amino acids which may be basic or acidic, formic acid, acetic acid, citric acid, tartaric acid, fumaric acid and oxalic acid.

It is noted that in this disclosure, terms such as “comprises”, “comprised”, “comprising”, “contains”, “containing” and the like can have the meaning attributed to them; e.g., they can mean “includes”, “included”, “including” and the like. Terms such as “consisting essentially of and “consists essentially of” have the meaning attributed to them, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of and “consisting of have the meaning ascribed to them; namely, that these terms are closed ended.

In a first aspect, the present invention provides an agent for use in medicine, wherein the agent comprises a compound of Formula (I):

B-L-B′

wherein:
B and B′ are independently selected from groups of formula B-I as follows:

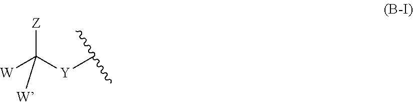

wherein:
Z is selected from —COOH, —CH₂COOH, —PO(OH)(OR¹), or —CH₂PO(OH)(OR¹),
wherein R¹ is a phosphate protecting group;
W is an alicyclic amine group having from 5 to 12 carbon atoms and at least one amine nitrogen atom;
W′ is H, or W′ is linked to W to form said alicyclic amine group; and
Y is selected from —NH—, —N(CH₃)—, —CH₂—, —NHCO—, —CH₂CONH—, —CONH—, —CH₂NHCO—, or —NHCH₂—; and
L is a linker group selected from: a direct bond; a saturated or unsaturated chain of from 1 to 12 carbon atoms in which from 1 to 4 of the carbon atoms are optionally replaced by O or S, and wherein the chain is optionally substituted by one or more groups selected from halogen, C1-C6 alkyl, C2-C6 alkenyl, C6-C12 (hetero)aryl, C6-C12 (hetero)arylC1-C4alkyl, or C1-C6 alkoxy; or L is a group of formula -L¹-Cy-L²- wherein Cy is a (hetero)aryl or (hetero)cycloalkyl group and L¹ and L² are independently selected from a direct bond or C1-C4 alkenyl groups in which one or two of the carbon atoms are optionally replaced by O or S, including individual stereoisomers thereof, stereoisomer mixtures thereof, and pharmaceutically acceptable salts, solvates, prodrugs or derivatives thereof.

It has been found that the above bivalent ligand compounds of Formula (I) are avidly bound by human CRP in vitro and in vivo, forming stable complexes of pairs of native pentameric CRP molecules cross-linked by up to 5 ligand molecules. The ligand binding pockets of each CRP protomer are blocked, and the whole binding (B) face of each CRP pentamer is fully occluded in this complex so that CRP cannot mediate tissue damaging action in vivo. Furthermore, dissociation of the individual, non-covalently associated, protomers of native CRP from within the CRP-ligand complex is completely inhibited under physiological conditions.

Suitably, the ligand groups B and B′ are the same. In these embodiments, the linker group L may also be symmetrical, whereby the compound of Formula (I) is a palindromic compound.

Suitably, Z is —COOH. In other embodiments, Z is —PO(OH)(OR¹), wherein R¹ is H, or preferably a phosphate protecting group. Examples of phosphate protecting groups include C1-C7 alkyl groups, C1-C7 alkenyl groups, or a C5-C6 aryl group linked to the phosphate through a C1-C4 alkylene group (i.e. C5-C6arylC1-C4 alkyl groups), any of which may optionally substituted with one or more halogen, —CN, or nitro groups. A benzyl group, diphenylmethyl group, triphenylmethyl group, 1-phenethyl group or 2-phenethyl group is suitable, and a benzyl group is especially suitable.

Suitably, the alicyclic amine group defined by W (or by W and W′) has one or more amine nitrogens in the ring. The amine group may then be secondary, tertiary or quaternary amine. Suitably, the amine group is a tertiary or quaternary amine group. The alicyclic amine may be a monocyclic amine group such as a piperidine, a pyrrolidine, a piperazine, a pyrimidine or a morpholine. Or the alicyclic amine may be a bicyclic amine group, such as an aza or diaza bicyclic [2.2.2], [2.2.1] or [3.2.1] bicyclic group. In any of these alicyclic groups the amine nitrogen may be alkylated with one or more C1-C4 alkyl groups to provide a tertiary or quaternary amine group in the ring. The alicyclic amine group may be linked to the rest of moiety B or B′ through any position on the alicyclic ring, including through the amine nitrogen. In embodiments, the alicyclic amine group is linked to the rest of the moiety B or B′ through a carbon atom located β or γ to the amine nitrogen. The alicyclic group may optionally be substituted with from 1 to 3 groups selected from halo, C1-C4 alkyl and C1-C4 alkoxy. In embodiments, the group W is quinuclidin-3-yl, quinuclidin-4-yl, N-methylpyrollidone-3-yl or N-methylpiperidine-4-yl.

In embodiments, the groups B and/or B′ are selected from the following groups B-II to B-XIII:

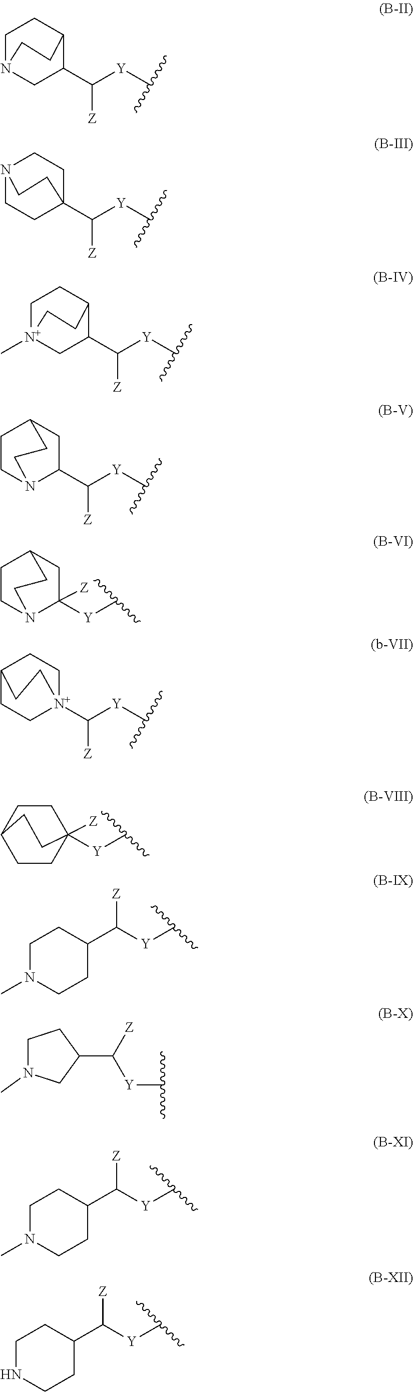

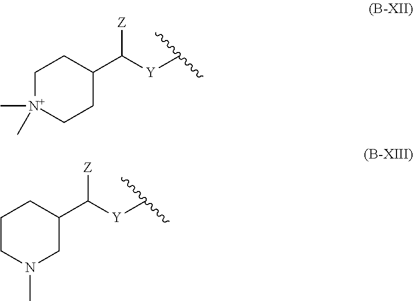

In more specific embodiments, the groups B and/or B′ are selected from the following groups B-XIV to B-XXI:

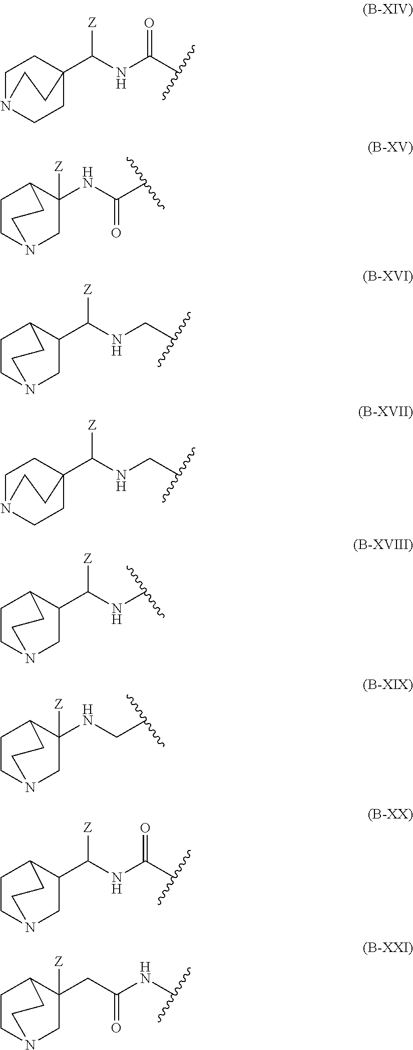

wherein Z is as defined in claim 1, preferably wherein Z is —COOH or —PO(OH)OR¹, wherein R¹ is a phosphate protecting group as defined above, suitably wherein R¹ is benzyl (C₆H₅CH₂—).

In these embodiments, the groups B and B′ are suitably selected from the groups B-XVI and B-XX above, in particular B-XX.

The stereochemistry at the chiral quinuclidine carbon is suitably R as shown above, but may be S. The stereochemistry at the carbon atom alpha to the quinuclidine ring is likewise preferably R. Suitably, when B and B′ are the same, they also have the same stereochemistry. All stereoisomers of the disclosed compounds, and mixtures thereof, are encompassed in the present disclosure.

Suitably, the linker group L is selected from a direct bond, a saturated or unsaturated alkylene or alkenylene chain of from 1 to 8 carbon atoms wherein the chain is optionally substituted by one or more C1-C4 alkyl groups or phenyl groups, or a linker group selected from one of L-I to L-IV as follows:

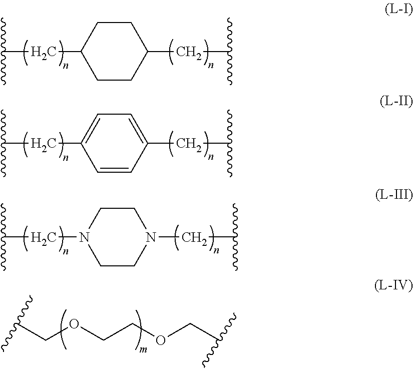

wherein n and m are 0, 1 or 2.

In these embodiments, the linker group L is suitably selected from a direct bond, an alkylene (—CₙH₂ₙ—) or alkenylene (—CₙH₂ₙ₋₂—) chain of 2, 4, 6 or 8 carbon atoms, or a linker group selected from one of L-V to L-VIII as follows:

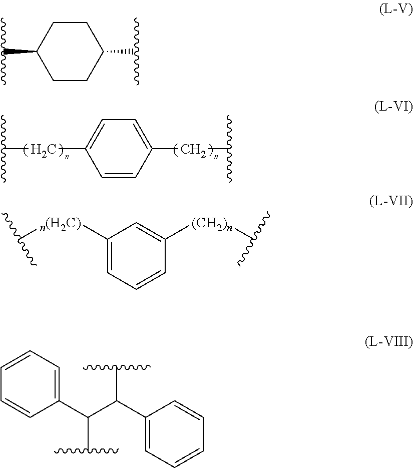

wherein n is 0 or 1.

In embodiments, the compound of Formula (I) is has the following Formula (II):

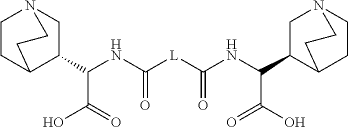

or the following Formula (III):

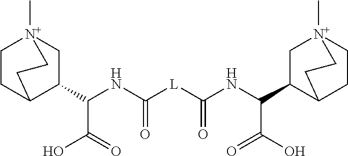

wherein L is a direct bond or a linker group of formula —(CH₂)ₙ— wherein n is from 1 to about 8, preferably wherein L is a direct bond or a linker group of formula —(CH₂)ₙ— wherein n is 2, 4, 6 or 8.

Specific compounds of Formulas (II) and (III) disclosed herein are as follows:

Compound P1d-0 having Formula (II) with L=direct bond;

Compound P1d-1 having Formula (II) with n=1;

Compound P1d-2 having Formula (II) with n=2;

Compound P1d-3 having Formula (II) with n=3;

Compound P1d-4 having Formula (II) with n=4;

Compound P1d-5 having Formula (II) with n=5;

Compound P1d-6 having Formula (II) with n=6;

Compound P1d-7 having Formula (II) with n=7;

Compound P1d-8 having Formula (II) with n=8; and

Compound P1d-4A having Formula (III) with n=4.

In embodiments, the present invention provides an agent for use in medicine, wherein the compound of Formula (I) comprises, consists essentially of, or consists of a compound of Formula (IV):

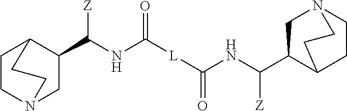

wherein:
Z are independently selected from —COOH, —CH₂COOH, —PO(OH)(OR¹), or —CH₂PO(OH)(OR¹), wherein R¹ is a phosphate protecting group; and
L is a linker group selected from:
   a direct bond;
   —CH₂CH₂— or —CH═CH— (preferably trans-CH═CH—), optionally substituted by one or more groups selected from halogen, hydroxy, trifluoromethyl, C1-C4 alkyl, C1-C4 alkoxy, C2-C4 alkenyl, or C6-C12 (hetero)aryl;
   an aryl linker group Ar; or
   a group of Formula (VI):

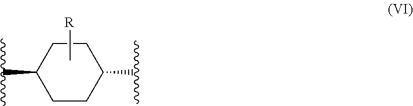

wherein R represents one, two or three optional substituents selected from halogen, hydroxy, trifluoromethyl, C1-C4 alkyl, C1-C4 alkoxy, C2-C4 alkenyl
including individual stereoisomers thereof, stereoisomer mixtures thereof, and pharmaceutically acceptable salts, solvates, prodrugs or derivatives thereof.

Suitably, the groups Z are independently selected from —COOH and —PO(OH)OR¹. In these embodiments, suitably either both groups Z are —COOH or both groups Z are PO(OH)OR¹. R¹ is preferably benzyl.

In these embodiments, the linker group L is suitably an aryl linker group Ar. The Ar linker group is suitably a monocyclic, bicyclic, or fused bicyclic aryl group optionally containing 1, 2 or 3 hetero atoms in the aromatic ring(s), the hetero atoms suitably being selected from N or S. The Ar linker group suitably contains from 4 to 12 carbon atoms in the aromatic rings (i.e. excluding carbon atoms in optional substituent groups). The aromatic ring(s) of the Ar group are linked to the palindromic end groups of the compounds of Formula (I) through amide bonds as shown in Formula (I). Suitably, the bond angle between the two Ar—CO bonds is about 180 degrees. Thus, for example, where Ar is a single six-membered aromatic ring such as a phenyl group, the bonds are suitably located para (1,4) on the ring. It appears that the resulting conformational relationship positions the quinuclidinyl end groups appropriately for binding to respective receptors in the CRP.

In embodiments, the Ar group is selected from 1,4-phenyl, 2,6-naphthyl or 4,4′-biphenyl, or groups of the same ring system containing 1, 2 or 3 heteroatoms in the ring(s), (e.g. 2,6-pyridyl instead of 1,4-phenyl). In each case, the aromatic rings may be substituted with one or more substituent groups R as defined below.

In these embodiments, the linker group Ar may be selected from the group consisting of the following general Formulae Ar-I to Ar-VI:

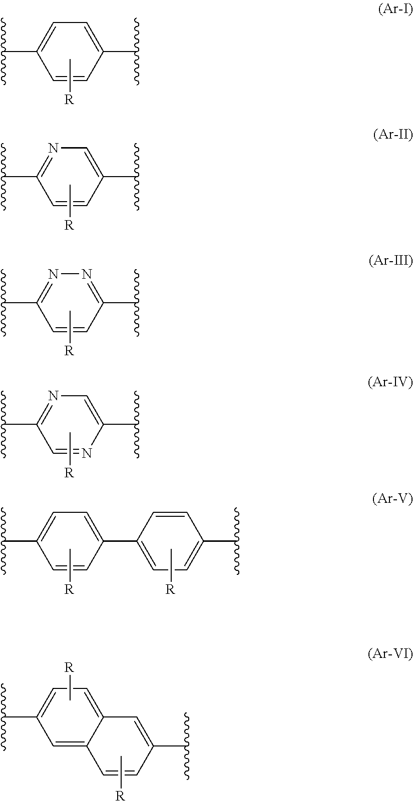

wherein R represents one or more optional substituents on the aryl ring(s). Suitably, R may be selected from halogen, hydroxy, cyano, —CONH₂, or C1-C5 (cyclo)alkyl or C1-C5 (cyclo)alkoxy wherein the alkyl groups are optionally substituted with a phenyl group (e.g. wherein R is —O-benzyl) or with one or more halogen atoms, for example trifluoromethyl. More suitably, R may be C1-C4 alkyl or C1-C4 alkoxy, for example methyl. Suitably, there are 0, 1 or 2 R substituents on the aryl linker, more suitably 0 or 1 R substituents, and in some cases no R substituents. In specific embodiments, the Ar linker group is a 1,4-phenyl linker group having 0, 1 or 2 R substituents.

In embodiments, the aryl linker group Ar is selected from the group consisting of groups having formulae Ar-VII to Ar-XVI.

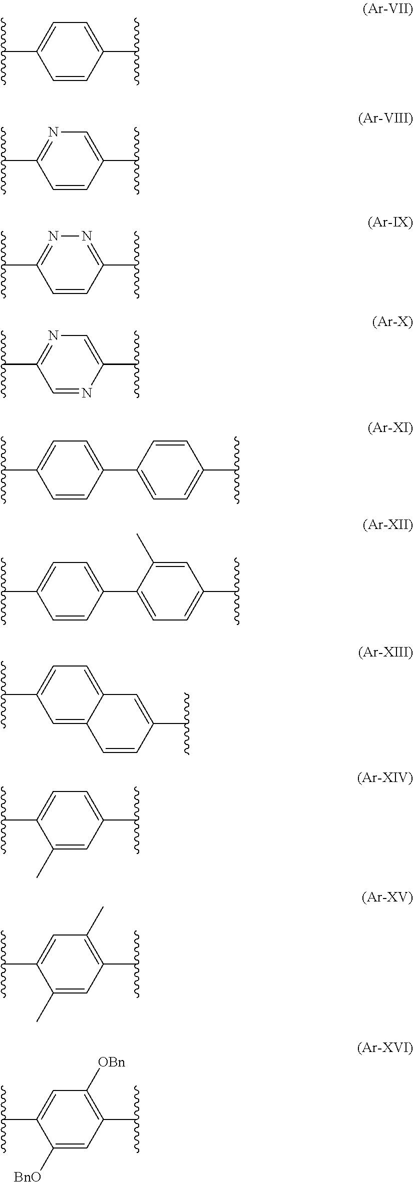

In alternative embodiments, the linker group L is selected from one of the groups L-I or L-II:

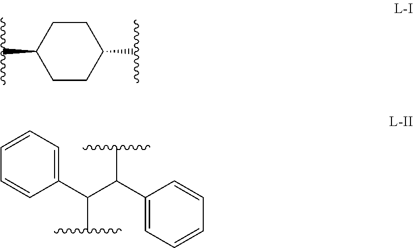 [SMILES: *[C@H]1CC[C@H](*)CC1 ; *C(c1ccccc1)C(*)c1ccccc1]

Suitably, the compound of Formula (IV) has the following Formula (V), also referred to herein as Compound P2B-D:

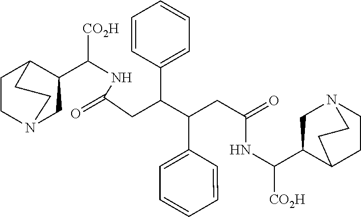 [SMILES: O=C(CC(c1ccccc1)C(CC(=O)NC(C(=O)O)[C@H]1CN2CCC1CC2)c1ccccc1)NC(C(=O)O)[C@H]1CN2CCC1CC2]

or the following Formula (VI), also referred to herein as Compound P3A-C (Bn in the structure below represents a benzyl group):

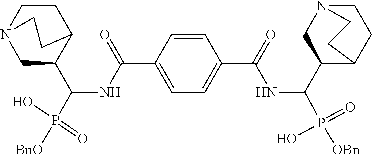

wherein Bn represents a benzyl group;
or the following Formula (VII), also referred to herein as Compound P2B-H:

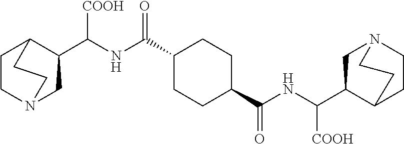 [SMILES: O=C(O)C(NC(=O)[C@H]1CC[C@H](C(=O)NC(C(=O)O)[C@H]2CN3CCC2CC3)CC1)[C@H]1CN2CCC1CC2]

Most suitably, the compound of Formula (IV) has Formula (VIII), also referred to herein as P2B-B or APL-2191:

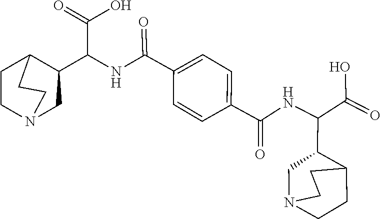 [SMILES: O=C(NC(C(=O)O)[C@H]1CN2CCC1CC2)c1ccc(C(=O)NC(C(=O)O)[C@H]2CN3CCC2CC3)cc1]

In particular the R,R,R,R stereoisomer thereof:

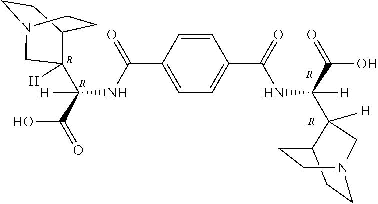

The compounds of Formula (IV) are suitably R,R,R,R stereoisomers. The other stereoisomers of this structure are thought to have lesser activity. The S,S,S,S isomer is thought to be the most active alternative stereoisomer.

Suitably, the diastereomeric purity of the (R,R,R,R) stereoisomer is at least about 50% by weight, suitably at least about 60%, more suitably at least about 75%, still more suitably at least about 90%, and most suitably at least about 98% in the agents of the invention. That is to say, the amount of the (R,R,R,R) stereoisomer suitably exceeds the amount of all other stereoisomers of this compound present in the agent. Most suitably, at least about 98% by weight of all stereoisomers of this compound present in the agent is the R,R,R,R stereoisomer.

Crystalline or dissolved forms of the compounds of Formula (I) that comprise both an alkylamino group (such as quinuclidinyl) and a —COOH group (e.g. when Z is —COOH) may exist in a zwitterionic form (COO− QNH+), and such zwitterionic forms are hereby encompassed in the definitions of Formula (I). Likewise, the definitions herein encompass all crystalline forms and polymorphs of the said compounds.

In embodiments, the compound of Formula (I) has the following Formula (IX), also referred to herein as Compound P2B-G:

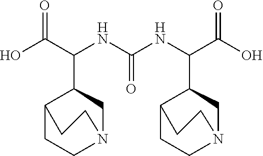 [SMILES: O=C(NC(C(=O)O)[C@H]1CN2CCC1CC2)NC(C(=O)O)[C@H]1CN2CCC1CC2]

or the following Formula (X), also referred to herein as Compound P2B-E:

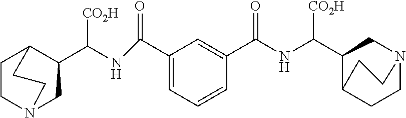 [SMILES: O=C(NC(C(=O)O)[C@H]1CN2CCC1CC2)c1cccc(C(=O)NC(C(=O)O)[C@H]2CN3CCC2CC3)c1]

or the following Formula (XI), also referred to herein as Compound P2B-C:

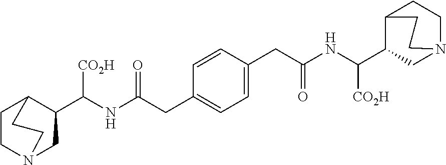 [SMILES: O=C(Cc1ccc(CC(=O)NC(C(=O)O)[C@H]2CN3CCC2CC3)cc1)NC(C(=O)O)[C@H]1CN2CCC1CC2]

or the following Formula (XII), also referred to herein as Compound P5A-B:

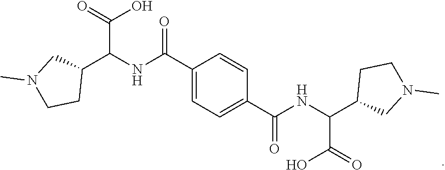

or the following Formula (XIII), also referred to herein as Compound P3A-C (Bn in the structure below represents a benzyl group):

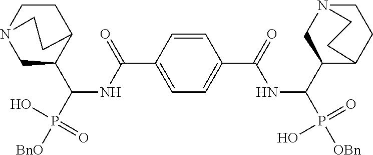 [SMILES: O=C(NC([C@H]1CN2CCC1CC2)P(=O)(O)OCc1ccccc1)c1ccc(C(=O)NC([C@H]2CN3CCC2CC3)P(=O)(O)OCc2ccccc2)cc1]

In another aspect, the present invention provides an agent for use in medicine comprising a compound of Formula (XII):

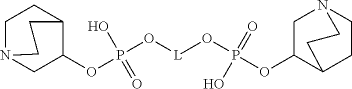

wherein L is a linker group as defined above in relation to the first aspect of the invention. Suitably, L is a linear or branched alkylene group of formula —CₙH₂ₙ— wherein n is from 1 to about 12, or a linear or branched alkenylene group of formula —CₙH₂ₙ₋₂— wherein n is from 1 to about 12. More suitably, L is a linear alkylene group of formula —(CH₂)ₙ— wherein n is from 1 to about 12, still more suitably wherein n is an even number from 2 to about 12, for example wherein n is 2, 4, 6 or 8.

Specific compounds of Formulas (XII) disclosed herein are as follows:

Compound PK-025 having Formula (XII) wherein L=—(CH₂)ₙ— with n=6;

Compound PK-026 having Formula (XII) wherein L=—(CH₂)ₙ— with n=7;

Compound PK-023 having Formula (XII) wherein L=—(CH₂)ₙ— with n=8;

Compound PK-028 having Formula (XII) wherein L=—(CH₂)ₙ— with n=9;

Compound PK-027 having Formula (XII) wherein L=—(CH₂)ₙ— with n=10;

Compound PK-033 having Formula (XII) wherein L=—CH₂C(CH₃)₂(CH₂)₄C(CH₃)₂CH₂—;
and Compound PK-032 having Formula (XII), wherein L=—CH₂C(CH₃)₂CH₂CH═CHCH₂C(CH₃)₂CH₂—

The specific stereochemistries disclosed herein are preferred. Where no stereochemistry is disclosed, the most preferred stereochemistry has not been determined with certainty. In embodiments, the preferred stereoisomer is the stereoisomer having the structure and/or properties (e.g. chromatographic elution rate) of the stereoisomers produced in the embodiments exemplified in the experimental section below. However, the invention also encompasses any stereoisomer, enantiomer or geometric isomer of the agents/compounds disclosed herein, and mixtures thereof.

Suitably, the compound of Formula (I) or Formula (IV) is an inhibitor of human C-reactive protein (CRP) having an IC₅₀ of about 200 μM or less as determined by the Roche immunoturbidimetric assay as described hereinbelow in relation to the supplementary examples, preferably about 50 μM or less, more preferably about 20 μM or less, still more preferably about 10 μM or less, or about 5 μM or less, or about 2 μM or less or less, or about 1 μM or less.

In a further aspect, the present invention provides an agent according to the invention for use in the treatment or prevention of a medical condition mediated by CRP. In another aspect, the present invention provides the use of an agent according to the first aspect of the invention for the manufacture of a medicament for treatment or prevention of a medical condition mediated by CRP.

The present invention further provides a method for treating a medical condition mediated by CRP in a patient in need thereof, comprising administering to the patient a therapeutic amount of an agent according to the invention, or a pharmaceutical composition according to the invention.

The medical condition mediated by CRP may be an ischemic condition, including for example acute myocardial infarction or stroke. The medical condition mediated by CRP may be an infection, a chronic inflammatory disease, or cancer cachexia. The inflammatory and/or tissue damaging condition may comprise acute coronary syndrome/unstable angina/plaque rupture/incipient atherothrombosis. Or the inflammatory and/or tissue damaging condition is selected from an infection, an allergic complication of infection, an inflammatory disease, ischemic or other necrosis, traumatic tissue damage and malignant neoplasia.

In embodiments, the condition is an infection selected from a bacterial infection including sepsis, a viral infection, a fungal infection and a parasitic infection, and complex conditions in which infection plays a role, such as chronic obstructive pulmonary disease (COPD). In embodiments, the condition is an allergic complication of infection selected from rheumatic fever, glomerulonephritis, and erythema nodosum leprosum. In embodiments, the condition is an inflammatory disease selected from rheumatoid arthritis, juvenile chronic (rheumatoid) arthritis, ankylosing spondylitis, psoriatic arthritis, systemic vasculitis, polymyalgia rheumatica, Reiter's disease, Crohn's disease and familial Mediterranean fever and other autoinflammatory conditions. In embodiments, the condition is tissue necrosis selected from myocardial infarction, ischaemic stroke, tumour embolization and acute pancreatitis. In embodiments, the condition is trauma selected from elective surgery, burns, chemical injury, fractures and compression injury. In embodiments, the condition is malignant neoplasia selected from lymphoma, Hodgkin's disease, carcinoma and sarcoma.

The present invention further provides a pharmaceutical composition comprising an agent according to the invention in admixture with one or more pharmaceutically acceptable excipients, diluents or carriers.

Pharmaceutical compositions may be formulated comprising an agent or a pharmaceutically acceptable salt, ester or prodrug thereof according to the present invention optionally incorporating a pharmaceutically acceptable carrier, diluent or excipient (including combinations thereof). By the term “pharmaceutically acceptable salt” is meant salts the anions or cations of which are known and accepted in the art for the formation of salts for pharmaceutical use. Acid addition salts, for example, may be formed by mixing a solution of the agent with a solution of a pharmaceutically acceptable, non-toxic acids, which include but are not limited to hydrochloric acid, oxalic acid, fumaric acid, maleic acid, succinic acid, acetic acid, citric acid, tartaric acid, carbonic acid or phosphoric acid. In especially suitable embodiments, the salt is a salt with HCl, in particular the 0.2HCl salt. Where the agent carries a carboxylic acid group, the invention also contemplates salts thereof, preferably non-toxic, pharmaceutically acceptable salts thereof, which include, but are not limited to the sodium, potassium, calcium and quaternary ammonium salts thereof.

Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Antioxidants and suspending agents may be also used.

The pharmaceutical compositions may be in the form of a prodrug comprising the agent or a derivative thereof which becomes active only when metabolised by the recipient. The exact nature and quantities of the components of such pharmaceutical compositions may be determined empirically and will depend in part upon the route of administration of the composition. Where appropriate, the pharmaceutical compositions of the present invention can be administered by inhalation, in the form of a suppository or pessary, topically (including ophthalmically) in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly, subcutaneously or intra-arterially.

For preparing solid compositions such as tablets, the principal active ingredient is mixed with a pharmaceutical carrier, e.g. conventional tabletting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of an agent, or a nontoxic, pharmaceutically acceptable salt thereof. The liquid forms in which the compositions of the present invention may be incorporated for administration orally or by injection include aqueous emulsions with edible oils such as cottonseed oil, sesame oil, coconut oil and peanut oil, as well as elixirs and similar pharmaceutical vehicles. Suitable dispersing or suspending agents for aqueous suspension include synthetic and natural gums such as tragacanth, acacia, alginate, dextran, sodium carboxymethylcellulose, methylcellulose, polyvinyl-pyrrolidone and gelatin.

For parenteral administration, the compositions may be best used in the form of a sterile aqueous solution which may contain other substances, for example buffers to adjust pH, or enough salts or monosaccharides to make the solution isotonic with blood. For buccal or sublingual administration the compositions may be administered in the form of tablets or lozenges which can be formulated in a conventional manner. For convenience of use, dosages according to the present invention are preferably administered orally but this will depend on the actual drug and its bioavailability.

Use of the compounds of the present invention aims to saturate with the ligand drug all circulating and other soluble CRP molecules in the body. The daily dose of drug required is therefore suitably that which provides at least about 1 mol of drug, more suitably at least about 5 mol of drug per mol of native pentameric CRP to be complexed.

The precise form of pharmaceutical composition and dosage thereof may also be dependent on the subject to be treated, including body weight, route of administration and disease conditions. These would be determined as a matter of routine by the skilled addressee.

### EXAMPLES

### I. Reagents and Methods

### 1. Reagents

(i) Human CRP was isolated, purified and characterised as previously reported (5). Rat CRP was isolated and purified as described (6). All preparations used herein were ≥95% pure. Stock solutions were stored frozen at −80° C. When required, stock CRP was thawed at 37° C. and working dilutions thereof prepared that were kept at 4° C. for the duration of an experiment. CRP concentration was determined spectrophotometrically (Beckman Coulter DU 650) in quartz cuvettes with a 1 cm light path, by measuring A₂₈₀ after correction for absorbance at 320 nm (light scattering) and using the 1% measured absorption coefficient A_{1 cm}^{1%}=17.5 for human CRP (7); an assumed value of 17.0 was used for rat CRP based on the earlier value measured for human CRP by interferometry (8)

(ii) ¹²⁵I-labelled CRP was freshly prepared from carrier-free Na ¹²⁵I (Perkin Elmer) and stock CRP by the N-bromosuccinimide method, and evaluated, as described (9). Specific activity was typically 12.8 MBq/nmol of pentamer.

(iii) Synthetic methods for the CRP-binding compounds are given below. A stock solution of each CRP-binding compound was prepared at 10 mM concentration in TC buffer and stored frozen at −30° C.; working dilutions for the experiments were prepared from the same stock solution. Ligand purity, for relative data analysis purposes, was assumed to be 100% unless stated otherwise. All compounds tested were soluble under the conditions used. A highly purified standard (>99.99%) of BPC8 was prepared by Almac. Pure batches of BPC8 and Pk-023 were also produced by Peakdale.

(iv) BPC8 for in vivo infusion was prepared in sterile pyrogen-free water at 366 mg/ml by Carbogen.

(v) Unless stated otherwise, all reagents and in vitro assays were conducted in 10 mM Tris, 2 mM CaCl₂, 140 mM NaCl, 0.1% w/v NaN₃, pH 8.0 (TC buffer) prepared in type 1 ultrapure water (Millipore Integral 10). TC buffer without azide was used for all in vivo studies

### 2. Immobilised Phosphocholine Plate Assay

(i) Phosphocholine was immobilised on Greiner NHS-activated microtitre plates by incubation with 4-aminophenyl-1-phosphocholine. Remaining active sites were blocked by incubation with 4% w/v BSA in TC buffer (TCB).

(ii) Human CRP at 1 μg/ml in TC buffer was spiked with ¹²⁵I-labelled CRP to provide 10⁶ cpm/ml.

(iii) Compounds were prepared from up to 10 mM stock solutions by dilution in TC buffer to 0.5 mM (labelled S1). Serial dilutions were prepared by diluting 100 μl ligand solution with 200 μl TC buffer (S2-S10). A ligand-free control was also used (SO, TC only).

(iv) A 100 μl volume of CRP solution was incubated with 10 μl of each compound (S1-S10, SO) for 1 h at room temperature without stirring, to provide final ligand concentrations across the range 45.5 μM-6.9 nM with a ligand:CRP monomer molar ratio of 1151-0.06. In some cases, single fixed ligand concentrations were also assayed in comparison with PC.

(v) A 100 μl volume of each CRP-ligand mixture was added to the plate wells and incubated for 2 h at room temperature. Solutions were then discarded and the plates washed with TCB buffer. Bound radioactivity was measured (1 min counts) using a gamma counter (Perkin Elmer Wizard 2470).

(vi) Data were plotted with Sigmaplot v14 software using the 4-parameter logistic curve y=min+(max−min)/(1+(x/EC₅₀)⁻Hill slope) to calculate IC₅₀.

### 3. Roche Immunoturbidimetric Assay: Pure CRP

(i) Human CRP at ˜90 μg/ml (3.9 μM monomer or 0.78 μM pentamer) in TC buffer was prepared from a stock solution; a 75 μl aliquot was used in the assay.

(ii) Compounds were prepared from stock solutions at up to 10 mM in TC (labelled S1). They were serially diluted with TC buffer (100 μl ligand+200 μl TC) to provide up to 9 dilutions, S2-S10. A TC buffer control (S0) was included in each assay.

(iii) A 15 μl volume of each ligand solution was incubated with 75 μl of CRP for 1 h at 37° C. The final concentrations were CRP pentamer=0.73 μM, ligands S1-S10=625-0.03 μM, corresponding to ligand:CRP pentamer ratios of 853-0.04.

(iv) CRP concentrations were measured by the Roche microparticle-enhanced immunoturbidimetric assay (10) on the COBAS MIRA autoanalyser, and results expressed as a percentage of the CRP control.

(v) Data were plotted using Sigmaplot (V14) using the 4 parameter logistic curve y=min+(max−min)/(1+(x/EC₅₀)⁻Hill slope) to calculate EC₅₀.

(vii) All compounds were assayed in comparison with a highly purified preparation of BPC8, that was prepared by Carbogen AMCIS AG, dissolved in sterile water and diluted into TC buffer as required.

### 4. Roche Immunoturbidimetric Assay: CRP in Acute Phase Human Serum

(i) Pooled whole acute phase human serum (APHS) was thawed from storage at −30° C. and clarified by centrifugation before the CRP concentration was determined by the Roche immunoturbidimetric assay run on the Roche COBAS MIRA instrument and by the Dade Behring/Siemens immunonephelometric assay run on the BN II instrument (11).

(ii) Serial 10-fold dilutions of CRP-binding compounds, and phosphocholine, in TC buffer were prepared from stock solutions of concentration up to 10 mM, using positive displacement pipettes (Anachem Microman series). Due to the limited supply of the human serum pool used, only four 10-fold dilutions of each compound were studied.

(iii) A 10 μl aliquot of each compound dilution was added to a 290 μl aliquot of neat APHS in autoanalyser tubes in the presence or absence of 10 mM di-tetra EDTA; replicate tubes were prepared where possible. Tube contents were mixed then incubated at 37° C. for 30 min.

(iv) Tube contents were mixed again, then the CRP concentration was measured immediately by the Roche assay to assess inhibition of CRP recognition, and by the BN II assay to determine total CRP concentration.

(v) Dose-response curves for Roche assay results were plotted by non-linear least squares analysis using ORIGIN v7.0 (MicroCal LLC) to calculate IC₅₀.

### 5. Size Exclusion Chromatography (SEC)

(i) Ligands were diluted to 0.5 mM in TC buffer from up to 10 mM stocks. Serial dilutions were prepared as required.

(ii) Ligands (10 μl of 0.5 mM solution and serial dilutions) were incubated for 1 h at room temperature with CRP (60 μl of 1 mg/ml, 2.6 nmol monomer, 520 pmol pentamer); this is about 10-fold ligand excess over CRP pentamer.

(iii) The complex was chromatographed on a Superdex200 column (10/300 GL; 24 ml bed volume), connected to the ÅKTA100 Explorer HPLC system, and equilibrated and eluted in TC buffer at 0.5 ml/min. Eluates were monitored at 280 nm and fractions (0.5 ml) collected as required.

### 6. X-Ray Crystallography

(i) Crystals of human CRP with and without ligands, were grown from by hanging-drop vapour diffusion in Linbro plates using previously known CRP crystallisation conditions (12).

(ii) X-ray data were collected to the resolutions mentioned below at the Diamond Light Source (Oxfordshire) beamlines, and processed with CCP4 programmes.

(iii) Each structure was solved by a molecular replacement method with the Phaser software package. Model building and refinement were performed with Phenix.

### 7. In Vivo Clearance of Ligand

(i) Ligand (0.5 mg/animal) dissolved in PBS or saline (200 μl) was injected intravenously into the tail vein of six male; 23-27 week old wild-type C57BL/6 mice. Up to two blood samples plus a terminal sample per mouse were collected at each time point up to 180 minutes.

(ii) Serum was prepared and ˜200 ng of internal standard (either ²H₄-BPC6 or BPC9) added. Serum proteins were precipitated with 4 volumes of either ice cold methanol or acetonitrile. The supernatant was dried under vacuum and re-dissolved in 0.1% v/v aqueous formic acid. (iii) Samples were chromatographed on a Phenomenex Aqua column using a gradient of 0.1% v/v formic acid-60% v/v acetonitrile in formic acid, and the eluate passed into the positive ion source of a Quattro II triple quadrupole mass spectrometer. The MRM transitions used were m/z 449.3→390.2 and 453.→394.2 (BPC6) and m/z 477.3→418.3, 491.3.3→432.3 (BPC8).

(iv) Ligand concentrations were measured against an extracted standard curve and were plotted using Sigmaplot (v14) and fitted to a 2-parameter exponential decay.

### 8. In Vivo Clearance of Preformed CRP/Ligand Complexes

(i) CRP at 1 mg/ml in azide-free TC buffer (containing, in some experiments, ˜10⁶ cpm/ml of ¹²⁵I-CRP) was incubated with a 5-fold molar excess of ligand. A control (ligand-free) sample was also prepared. Cross linking of CRP was confirmed by SEC in TC buffer of a small sample of complex.

(ii) Two groups of eight wild-type C57BL/6 mice (male; 23-27 weeks old) were used.

The complex or control (200 μl, 200 μg CRP) was injected into the tail vein of each mouse. A single timed collection of blood was taken from the tail vain of each mouse; a second terminal sample was also collected. This yielded 4 samples each at 30, 60, 120 and 180 min for control and treated groups.

(iii) Blood samples were weighed and CRP measured in the Roche assay and, in some cases by electroimmunoassay (4, 13). ¹²⁵I-CRP was measured by gamma counting (Perkin Elmer Wizard; protocol #1).

### 9. In Vivo Clearance of CRP/Ligand Complexes Generated In Vivo

(i) Two groups of 8 wild-type C57BL/6 mice (male; 23-27 weeks old) were used: group 1 (control) and group 2 (treated).

(ii) Both groups of animals were given 200 μl of human CRP (5 mg/ml in azide-free TC buffer) subcutaneously on day 1.

(iii) On day 2 (+18 h), control animals were given a 200 μl intravenous bolus of TC buffer. Treated animals were given a 200 μl iv bolus of ligand (prepared at 5 mg/ml in TC buffer).

(iv) One timed blood sample and a terminal blood sample were collected from each mouse into heparinised tubes and weighed. This generated data in quadruplicate at 4 time points, normally 30, 60, 120 and 360 minutes.

(v) ¹²⁵I radiolabel was measured (Perkin Elmer Wizard; protocol #1) in whole blood to quantify total CRP. In some cases, electroimmunoassay as above was also used to determine total CRP concentration.

(vi) Plasma was prepared and free CRP determined by Roche immunoassay.

### II. Synthesis of Ligand Compounds

### Abbreviations

### DMAP 4-Dimethylaminopyridine

### TEA Triethylamine

### DCM Dichloromethane

### EA Ethyl Acetate

KHMDS Potassium bis(trimethylsilyl)amide

### THF Tetrahydrofuran

TFA Trifluoroacetic acid

### PE Petroleum Ether

### DIPEA Disopropylethylamine

### DMF Dimethylformamide

### EtOAc Ethyl Acetate

### ACN Acetonitrile

### Procedure 1: Preparation of [(3S)-quinuclidin-3-yl] 4-bromobenzenesulfonate

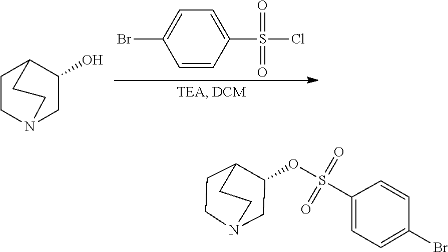 [SMILES: O[C@@H]1CN2CCC1CC2 ; O=S(=O)(Cl)c1ccc(Br)cc1 ; O=S(=O)(O[C@@H]1CN2CCC1CC2)c1ccc(Br)cc1]

To a solution of (3S)-quinuclidin-3-ol (2.00 g, 15.73 mmol, 1.00 eq), DMAP (19.21 mg, 157.30 gmol, 0.01 eq) and TEA (4.78 g, 47.19 mmol, 6.54 mL, 3.00 eq) in DCM (40.00 mL) was added 4-bromobenzenesulfonyl chloride (6.03 g, 23.60 mmol, 1.50 eq) at 0° C. The mixture was stirred for 16 hours at 20° C. The mixture was washed with sat. NaHCO₃ (100 mL). The sat. NaHCO₃ layer was extracted with EA (100 mL×2). The combined organic layers were dried over Na₂SO₄ and concentrated under reduced pressure to give a crude product as a yellow oil. The yellow oil was purified by chromatography on silica gel eluted with DCM:MeOH=30:1 to give [(3S)-quinuclidin-3-yl] 4-bromobenzenesulfonate (3.20 g, 8.32 mmol, 52.88% yield, 90% purity) as a yellow solid, which was analyzed by ¹HNMR.

¹H NMR: (400 MHz, CDCl₃) δ=7.81-7.57 (m, 4H), 4.65-4.49 (m, 1H), 3.04 (dd, J=8.4, 15.2 Hz, 1H), 2.89-2.48 (m, 5H), 1.93 (d, J=2.8 Hz, 1H), 1.80-1.71 (m, 1H), 1.61 (tdd, J=4.6, 9.5, 13.9 Hz, 1H), 1.47-1.22 (m, 2H).

### Procedure 2: Preparation of 2-(benzhydrylideneamino)-2-[(3R)-quinuclidin-3-yl]acetate

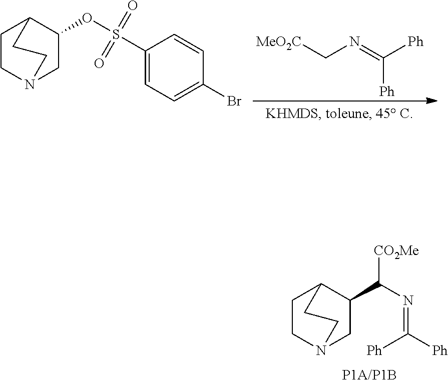 [SMILES: O=S(=O)(O[C@@H]1CN2CCC1CC2)c1ccc(Br)cc1 ; COC(=O)CN=C(c1ccccc1)c1ccccc1 ; COC(=O)C(N=C(c1ccccc1)c1ccccc1)[C@H]1CN2CCC1CC2]

The compounds P1A and P1B are stereoisomers having opposite stereochemistry (R or S) at the carbon atom attached to the quinuclidine ring. The absolute stereochemistry of the isomers are not known with certainty, but it is thought that P1A may have R stereochemistry at the alpha carbon.

To a solution of [(3S)-quinuclidin-3-yl] 4-bromobenzenesulfonate (3.10 g, 8.95 mmol, 1.00 eq) and methyl 2-(benzhydrylideneamino)acetate (3.86 g, 15.22 mmol, 1.70 eq) in toluene (62.00 mL) was added KHMDS (1 M, 12.53 mL, 1.40 eq) at 45° C. The mixture was stirred for 16 hours at 45° C. Sat. NH₄Cl (500 mL) was added to the reaction mixture. The mixture was extracted with EtOAc (300 mL×2). The combined organic layers were washed with saturated brine (200 mL), dried over Na₂SO₄ and concentrated to give a crude product. The crude product was purified twice by column chromatography on silica gel eluted with EtOAc:MeOH (NH₃, 7M)=50:1 to give 600 mg of unreacted bromobenzene sulfonate as a yellow solid, 450 mg of a mixture of unreacted bromobenzene sulfonate and P1A as a yellow oil, 590 mg of P1A as a yellow solid and 500 mg of a mixture of P1A and P1B as a yellow oil. 450 mg of the mixture of unreacted bromobenzene sulfonate and P1A and 500 mg of a mixture of P1A and P1B were purified by Prep-TLC (EtOAc:MeOH (NH₃, 7M)=10:1) to give 202.25 mg of P1A (91.7% purity, 98.6% ee.) as a yellow solid, 114.50 mg of P1B (97.7% purity, 94.3% ee.) as a yellow oil and 82.88 mg of a mixture of P1A and P1B (92.1% purity) as a yellow oil.

LCMS: m/z=363.2 (M+H⁺)

P1A: ¹H NMR: (400 MHz, CDCl₃) δ=7.66-7.58 (m, 2H), 7.52-7.45 (m, 3H), 7.43-7.31 (m, 3H), 7.19 (dd, J=1.6, 7.4 Hz, 2H), 4.22 (d, J=8.3 Hz, 1H), 3.73-3.68 (m, 3H), 3.17-3.03 (m, 1H), 2.92-2.82 (m, 2H), 2.64-2.51 (m, 3H), 1.77-1.55 (m, 3H), 1.52-1.40 (m, 1H), 1.37-1.25 (m, 1H)

P1B: ¹H NMR: (400 MHz, CDCl₃) δ=7.65-7.56 (m, 2H), 7.54-7.44 (m, 3H), 7.43-7.29 (m, 3H), 7.20 (dd, J=2.9, 6.4 Hz, 2H), 4.08 (d, J=10.0 Hz, 1H), 3.76-3.72 (m, 3H), 3.24-3.09 (m, 1H), 3.00-2.80 (m, 2H), 2.61-2.38 (m, 3H), 1.81-1.58 (m, 3H), 1.28-1.15 (m, 1H), 1.12-1.00 (m, 1H)

Spectra:

### Procedure 3: Preparation of methyl 2-amino-2-[(3R)-quinuclidin-3-yl]acetate

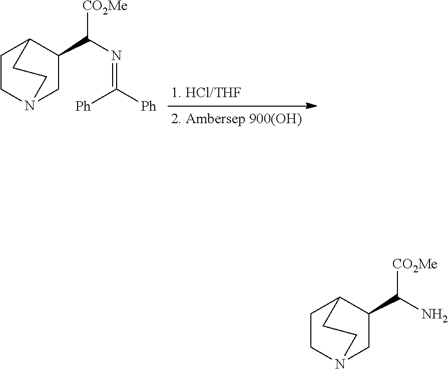 [SMILES: COC(=O)C(N=C(c1ccccc1)c1ccccc1)[C@H]1CN2CCC1CC2 ; COC(=O)C(N)[C@H]1CN2CCC1CC2]

To a solution of the above-prepared P1A stereoisomer of methyl 2-(benzhydrylideneamino)-2-[(3R)-quinuclidin-3-yl]acetate, (390.00 mg, 1.08 mmol, 1.00 eq) in THF (6.00 mL) was added HCl (12 M, 780.09 μL, 37% purity, 8.70 eq) at 0° C. The reaction mixture was stirred for 1 hour at 0° C. The mixture was concentrated to remove THF. To the mixture was added methyl tertiary butyl ether (20 mL) and water (20 mL). The aqueous layer was concentrated under reduced pressure to give methyl 2-amino-2-[(3R)-quinuclidin-3-yl]acetate (250.00 mg, crude, 2HCl) as a yellow solid. Ambersep 900(OH) resin (2.6 g) was washed with MeOH (2×20 mL). A slurry of the resin in MeOH (12 mL) was added to a stirred solution of methyl 2-amino-2-[(3R)-quinuclidin-3-yl]acetate (250.00 mg, 921.90 gmol, 1.00 eq, 2HCl) in MeOH (12 ml). The reaction mixture was stirred for 1 hour at 20° C. The mixture was filtered. The filtrate was concentrated to give a residue. The residue was dissolved into CHCl₃ (20 mL) and filtered. The filtrate was concentrated to give a yellow oil, which was co-evaporated with toluene (20 mL×2) to give methyl 2-amino-2-[(3R)-quinuclidin-3-yl]acetate (150.00 mg, 696.06 gmol, 75.50% yield, 92% purity) as a yellow oil. The product may be purified by prep-HPLC (TFA) to obtain a colorless oil for use in subsequent synthesis steps.

¹H NMR (400 MHz, CDCl₃) δ=3.78-3.65 (m, 3H), 3.35 (d, J=10.5 Hz, 1H), 3.22-3.11 (m, 1H), 2.91-2.80 (m, 5H), 1.95 (s, 1H), 1.88-1.39 (m, 6H)

LCMS: RT=4.040, m/z=199.2 (M+H+)

The product may be purified by prep-HPLC (TFA) to obtain a colorless oil for use in subsequent synthesis steps.

### Example 1: Preparation of P1d-n Series Compounds_n (n=2, 3, 4, 5, 6, 7, 8)

Step 1

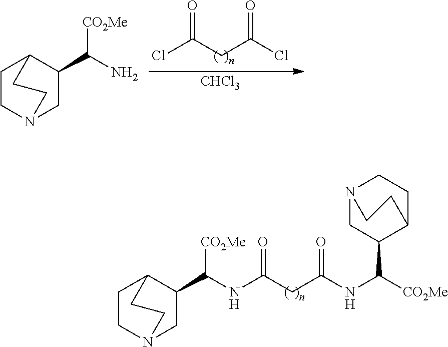

To a mixture of the above-prepared methyl 2-amino-2-[(3R)-quinuclidin-3-yl]acetate (100.00 mg, 504.39 gmol, 1.00 eq) in CHCl₃ (8.00 mL) was added 1,n- (n-alkyl) dicarbonyl dichloride (0.40 eq) in CHCl₃ (1.00 mL) at 25° C. The mixture was stirred for 16 hours at 25° C. under N₂ atmosphere. The mixture was concentrated to remove CHCl₃. To the bottle was added sat. NaHCO₃ (2 mL). The mixture was concentrated to give a white solid, which was washed with MeOH (50 mL). The MeOH layer was concentrated to give the crude product as a white solid.

Step 2

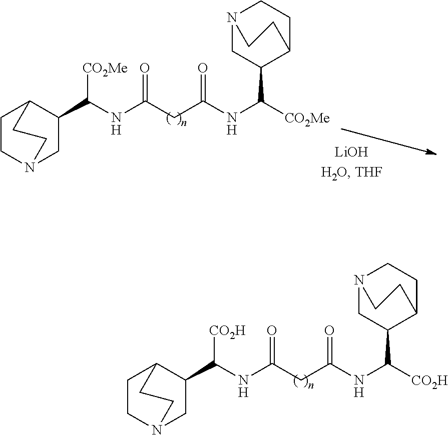 [SMILES: COC(=O)C(NC(=O)CC(=O)NC(C(=O)OC)[C@H]1CN2CCC1CC2)[C@H]1CN2CCC1CC2 ; O=C(CC(=O)NC(C(=O)O)[C@H]1CN2CCC1CC2)NC(C(=O)O)[C@H]1CN2CCC1CC2]

To a solution of the dimethyl ester produced in step 1 (1.00 eq) in water (2.00 mL) was added LiOH (0.5N in THF, 8.00 eq) at 20° C. The mixture was stirred for 16 hours at 20° C. The mixture was concentrated to remove THF. To the mixture was added water (5 mL) and HCl (1N) to pH=2. The mixture was concentrated to remove water to give a residue, which was washed with MeOH (20 mL×2). The MeOH layer was concentrated to give a crude product. The crude product was purified by Prep-HPLC to give P1d-n as a white solid.

P1d-2: ¹H NMR (400 MHz, D2O) 4.50-4.38 (m, 2H), 3.47 (br t, J=11.7 Hz, 2H), 3.37-3.17 (m, 8H), 3.00-2.88 (m, 2H), 2.65-2.44 (m, 6H), 2.21-2.04 (m, 4H), 2.01-1.80 (m, 6H)

LCMS: RT=4.54, m/z=451.3 (M+H⁺)

P1d-3: ¹H NMR (400 MHz, DEUTERIUM OXIDE) 4.40 (d, J=11.0 Hz, 2H), 3.45 (t, J=11.7 Hz, 2H), 3.33-3.15 (m, 8H), 2.92 (ddd, J=2.2, 7.0, 13.1 Hz, 2H), 2.52-2.38 (m, 2H), 2.36-2.22 (m, 4H), 2.19-2.02 (m, 4H), 1.98-1.75 (m, 8H)

LCMS: RT=4.80, m/z=465.4 (M+H⁺)

P1d-4: ¹H NMR (400 MHz, DEUTERIUM OXIDE) 4.40 (d, J=11.0 Hz, 2H), 3.45 (br t, J=11.7 Hz, 2H), 3.34-3.11 (m, 8H), 2.89 (ddd, J=2.3, 7.0, 13.1 Hz, 2H), 2.51-2.37 (m, 2H), 2.24 (br s, 4H), 2.17-2.02 (m, 4H), 2.01-1.80 (m, 6H), 1.51 (t, J=3.0 Hz, 4H)

LCMS: RT=5.08, m/z=479.4 (M+H⁺)

P1d-5: ¹H NMR (400 MHz, DEUTERIUM OXIDE) 4.38 (d, J=11.0 Hz, 2H), 3.44 (t, J=11.7 Hz, 2H), 3.37-3.10 (m, 8H), 2.88 (ddd, J=1.8, 7.0, 12.9 Hz, 2H), 2.52-2.35 (m, 2H), 2.22 (t, J=7.3 Hz, 4H), 2.16-2.03 (m, 4H), 2.01-1.77 (m, 6H), 1.51 (quin, J=7.4 Hz, 4H), 1.21 (td, J=7.6, 14.7 Hz, 2H)

LCMS: RT=5.57, m/z=493.1 (M+H⁺)

P1d-6: ¹H NMR (400 MHz, DEUTERIUM OXIDE) 4.40 (d, J=11.0 Hz, 2H), 3.46 (br t, J=11.4 Hz, 2H), 3.34-3.17 (m, 8H), 2.96-2.81 (m, 2H), 2.55-2.39 (m, 2H), 2.22 (t, J=7.3 Hz, 4H), 2.17-2.04 (m, 4H), 2.00-1.81 (m, 6H), 1.51 (t, J=6.8 Hz, 4H), 1.29-1.16 (m, 4H)

LCMS: RT=1.66, m/z=507.2 (M+H⁺)

P1d-7: ¹H NMR (400 MHz, DEUTERIUM OXIDE) 4.41 (d, J=11.0 Hz, 2H), 3.45 (br t, J=11.5 Hz, 2H), 3.36-3.13 (m, 8H), 2.89 (ddd, J=1.9, 7.0, 13.0 Hz, 2H), 2.54-2.39 (m, 2H), 2.22 (t, J=7.3 Hz, 4H), 2.16-2.02 (m, 4H), 2.01-1.78 (m, 6H), 1.57-1.44 (m, 4H), 1.27-1.15 (m, 1H), 1.27-1.15 (m, 5H)

LCMS: RT=2.163, m/z=521.3 (M+H⁺)

P1d-8: ¹H NMR (400 MHz, DEUTERIUM OXIDE) 4.41 (d, J=11.0 Hz, 2H), 3.45 (t, J=11.7 Hz, 2H), 3.35-3.14 (m, 8H), 2.96-2.82 (m, 2H), 2.54-2.37 (m, 2H), 2.22 (t, J=7.3 Hz, 4H), 2.15-2.01 (m, 4H), 2.01-1.78 (m, 6H), 1.50 (t, J=6.5 Hz, 4H), 1.20 (s, 8H)

LCMS: RT=2.281, m/z=535.3 (M+H⁺)

(Note: Compounds of the P1d-n series having n=0 or n=1 were also successfully made by the above synthetic method using oxalyl dichloride or propan-1,3-dicarbonylchloride, respectively, in step 1.)

### Example 2: Preparation of 2-[[4-[[-carboxy-[(3R)-quinuclidin-3-yl]methyl]carbamoyl]benzoyl]amino]-2-[(3R)-quinuclidin-3-yl]acetic acid (Compound P2B-B)

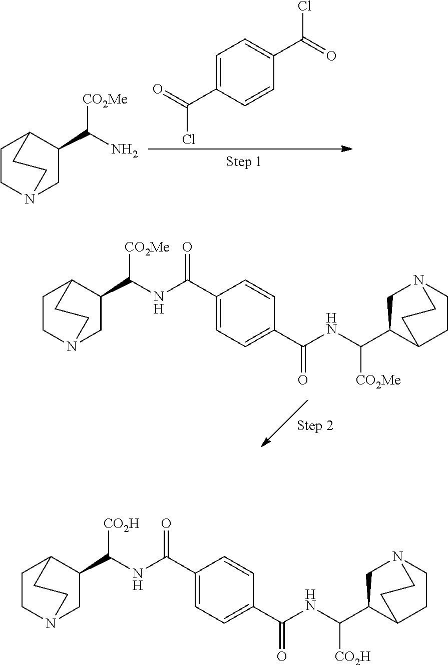 [SMILES: COC(=O)C(N)[C@H]1CN2CCC1CC2 ; O=C(Cl)c1ccc(C(=O)Cl)cc1 ; COC(=O)C(NC(=O)c1ccc(C(=O)NC(C(=O)OC)[C@H]2CN3CCC2CC3)cc1)[C@H]1CN2CCC1CC2 ; O=C(NC(C(=O)O)[C@H]1CN2CCC1CC2)c1ccc(C(=O)NC(C(=O)O)[C@H]2CN3CCC2CC3)cc1]

Step 1: To a solution of the above-prepared methyl 2-amino-2-[(3R)-quinuclidin-3-yl]acetate (100.00 mg, 504.39 gmol, 1.00 eq) in CHCl3 (8.00 mL) was added benzene-1,4-dicarbonyl chloride (51.20 mg, 252.19 gmol, 0.50 eq) at 30° C. The mixture was stirred for 16 hours at 30° C. The mixture was concentrated to give a crude product as a white solid. Compound methyl 2-[[4-[-2-methoxy-2-oxo-1-[(3R)-quinuclidin-3-yl]ethyl]carbamoyl]benzoyl] amino]-2-[(3R)-quinuclidin-3-yl]acetate (180.00 mg, crude, HCl) was obtained.

LCMS: m/z=527.5 (M+H⁺)

Step 2: To a solution of methyl 2-[[4-[[-2-methoxy-2-oxo-1-[(3R)-quinuclidin-3-yl]ethyl]carbamoyl]benzoyl]amino]-2-[(3R)-quinuclidin-3-yl]acetate (180.00 mg, 341.80 gmol, 1.00 eq) in THF (2.00 mL) was added a solution of LiOH (48.00 mg, 2.00 mmol, 5.86 eq) in H₂O (2.00 mL) at 25° C. The mixture was stirred for 1 hours at 25° C. The mixture was concentrated under reduced pressure to remove THF. To the mixture was added HCl (1N) to pH=3. The mixture was concentrated under reduced pressure to give a residue. The residue was dissolved in to MeOH (5 mL) and purified by prep-HPLC (TFA) to give 2-[[4-[[carboxy-[(3R)-quinuclidin-3-yl]methyl]carbamoyl]benzoyl]amino]-2-[(3R)-quinuclidin-3-yl]acetic acid (24.20 mg, 48.05 gmol, 14.06% yield, 99% purity) as a white solid, which was analysed by HNMR of UCL_P2B_B and LCMS of UCL_P2B_B.

¹H NMR (400 MHz, DEUTERIUM OXIDE) δ 7.77 (s, 4H), 4.62 (d, J=11.2 Hz, 2H), 3.50 (br t, J=10.9 Hz, 2H), 3.41-3.14 (m, 8H), 3.11-2.96 (m, 2H), 2.65-2.57 (m, 2H), 2.32-2.07 (m, 4H), 2.05-1.79 (m, 6H)

LCMS: RT=5.91, m/z=499.3 (M+H⁺)

### Example 3: Preparation of 2-[[2-[4-[2-[[carboxy-[(3R)-quinuclidin-3-yl]methyl]amino]-2-oxo-ethyl]phenyl]acetyl]amino]-2-[(3R)-quinuclidin-3-yl]acetic acid (Compound P2B-C)

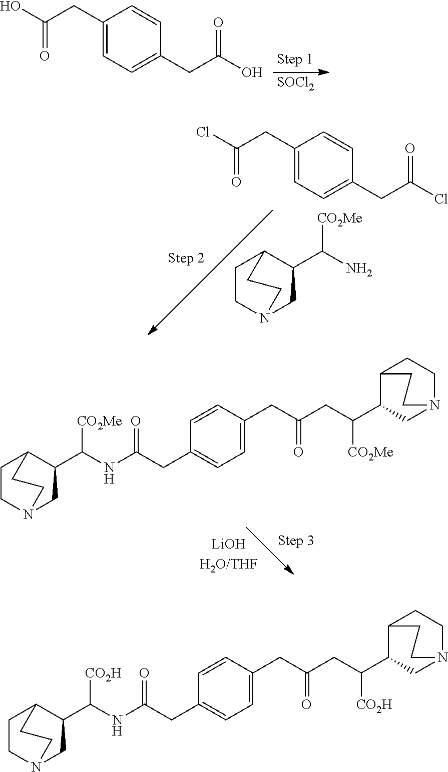

Step 1: To a solution of 2-[4-(carboxymethyl)phenyl]acetic acid (100.00 mg, 514.99 gmol, 1.00 eq) and DMF (3.76 mg, 51.50 gmol, 3.96 μL, 0.10 eq) in DCM (4.00 mL) was added SOCl2 (122.54 mg, 1.03 mmol, 74.72 μL, 2.00 eq) at 30° C. The mixture was stirred for 16 hours at 30° C. The mixture was concentrated under reduced pressure to give 2-[4-(2-chloro-2-oxo-ethyl)phenyl]acetyl chloride (100.00 mg, 419.77 gmol, 81.51% yield, 97% purity) as a yellow solid.

LCMS: RT=0.813, m/z=223.0 (M+H⁺)

Step 2: To a solution of the above-prepared methyl 2-amino-2-[(3R)-quinuclidin-3-yl]acetate (82.00 mg, 413.60 gmol, 1.00 eq) and TEA (83.70 mg, 827.20 gmol, 114.66 μL, 2.00 eq) in CHCl₃ (4.00 mL) was added a solution of 2-[4-(2-chloro-2-oxo-ethyl)phenyl]acetyl chloride (47.79 mg, 206.80 gmol, 0.50 eq) in CHCl₃ (1.00 mL) at 30° C. The mixture was stirred for 16 hours at 30° C. The mixture was concentrated under reduced pressure to a crude product as yellow oil.

LCMS: RT=0.990, m/z=555.3 (M+H⁺)

Step 3: To a solution of methyl 2-[[2-[4-[2-[[-2-methoxy-2-oxo-1-[(3R)-quinuclidin-3-yl]ethyl]amino]-2-oxo-ethyl]phenyl]acetyl]amino]-2-[(3R)-quinuclidin-3-yl]acetate (43.00 mg, 77.52 gmol, 1.00 eq) in H₂O (2.00 mL) was added a solution of LiOH (96.00 mg, 4.01 mmol, 51.71 eq) in THF (2.00 mL) at 30° C. The mixture was stirred for 24 hours at 30° C. The mixture was concentrated under reduced pressure to give a crude product as a yellow solid. The crude product was purified by prep-HPLC (TFA) to give 2-[[2-[4-[2-[[carboxy-[(3R)-quinuclidin-3-yl]methyl]amino]-2-oxo-ethyl]phenyl]acetyl]amino]-2-[(3R)-quinuclidin-3-yl]acetic acid (34.60 mg, 55.40 gmol, 71.47% yield, 96% purity, 2HCl) as a white solid, which was analysed by LCMS of UCL_P2B_C and HNMR of UCL_P2B_C.

¹H NMR (400 MHz, DEUTERIUM OXIDE) δ 7.20 (s, 4H), 4.41 (d, J=10.8 Hz, 2H), 3.62-3.52 (m, 4H), 3.45-3.33 (m, 2H), 3.31-3.11 (m, 8H), 2.82 (ddd, J=2.0, 7.1, 13.0 Hz, 2H), 2.55-2.40 (m, 2H), 2.11 (br d, J=2.2 Hz, 2H), 2.06-1.71 (m, 8H)

LCMS: RT=6.28, m/z=527.3 (M+H⁺)

### Example 4: Synthesis of Compound P2B-D

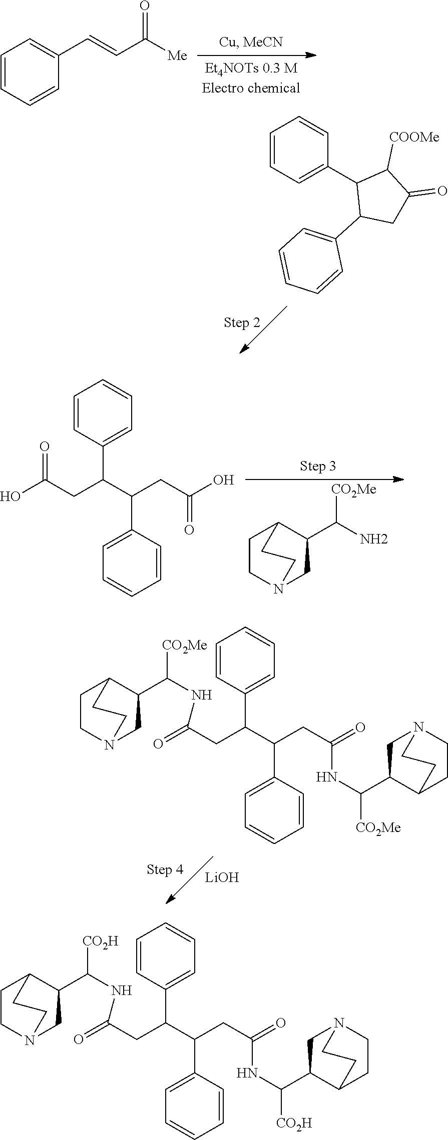

Step 2: A mixture of the bisphenyl cyclopentanone ester shown above (420.00 mg, 1.43 mmol, 1 eq) and NaOH (2.5 M, 42.24 mL, 74.00 eq) in dioxane (20.00 mL) and H₂O (20.00 mL) was stirred at 120° C. for 6 hrs. When the reaction was complete which was detected by TLC (PE:EtOAc=5:1), the organic solvent was concentrated under vacuum, then the product was washed with EtOAc (10 mL*2). The aqueous layer was acidified with 3N HCl to pH=3, then the mixture was filtered, the filter cake was washed with H₂O (5 mL*4), and dried over under vacuum to give a crude product 3,4-bisphenyl hexane-1,6-dioic acid (0.32 g, crude) as a white solid which was confirmed by ¹H-NMR

¹H-NMR (400 MHz, CD₃OD) 7.14-7.10 (m, 6H), 6.94-6.92 (m, 4H), 3.34-3.30 (m, 2H), 2.77-2.75 (m, 2H), 2.60-2.55 (m, 2H).

Step 3: To the solution of 3,4-bisphenyl hexane-1,6-dioic acid (80.00 mg, 268.16 gmol, 1 eq), the above-prepared methyl 2-amino-2-[(3R)-quinuclidin-3-yl]acetate (228.63 mg, 536.31 gmol, 2.00 eq, 2TFA) and DIPEA (138.63 mg, 1.07 mmol, 186.83 μL, 4.00 eq) in DMF (1.00 mL) and DCM (2.00 mL) was added HATU (224.32 mg, 589.95 gmol, 2.20 eq) at 0° C., after addition, the resulting mixture was stirred at 25° C. for 0.5 hr. When the reaction was complete, which was detected by LCMS, the mixture was concentrated by N₂ stream to give a crude product. The crude product was purified by prep-HPLC (TFA). The product with shorter retention time was assigned as Peak 1 product (50.00 mg, 64.70 gmol, yield: 24.13%, and was obtained as colorless oil which was confirmed by ¹H-NMR. The product with middle retention time was assigned as Peak 2 product (30.00 mg, 38.82 gmol, yield: 14.48%) and was obtained as a colorless oil and was confirmed by ¹H-NMR. The product with longest retention time was assigned as Peak 3 product (60.00 mg, 77.63 gmol, yield: 28.95%, and was obtained as a colorless oil and was confirmed by ¹H-NMR.

¹H-NMR (Peak 1) (400 MHz, CD₃OD) 7.16-7.07 (m, 6H), 6.98-6.96 (m, 4H), 4.41 (d, 2H, J=10.8 Hz), 3.69 (s, 6H), 3.46-3.44 (m, 2H), 3.31-3.29 (m, 2H), 3.25-3.10 (m, 4H), 3.10-3.00 (m, 2H), 2.80-2.76 (m, 4H), 2.62-2.61 (m, 2H), 2.22-2.21 (m, 2H), 2.08-2.05 (m, 2H), 2.03-1.93 (m, 6H), 1.76-1.75 (m, 4H).

The spectrum of the Peak 2 product was too complex to be analyzed.

¹H-NMR (Peak 3) (400 MHz, CD₃OD) 7.16-7.11 (m, 6H), 6.93-6.91 (m, 4H), 4.38 (d, 2H, J=11.2 Hz), 3.51 (s, 6H), 3.50-3.49 (m, 2H), 3.30-3.20 (m, 8H), 2.81-2.74 (m, 4H), 2.65-2.61 (m, 2H), 2.40-2.35 (m, 2H), 2.05-1.97 (m, 6H), 1.87-1.83 (m, 4H).

Step 4: To a solution of the Peak 1 compound (50.00 mg, 64.70 gmol, 1 eq, TFA) in MeOH (2.00 mL) and THF (2.00 mL) was added a solution of LiOH-H₂O (40.72 mg, 970.44 gmol, 15.00 eq) in H₂O (1.00 mL), the resulting mixture was stirred at 25° C. for 12 hrs. When the reaction was complete which was detected by LCMS the organic solvent was removed by N₂ stream to give a crude product. This was acidified by HCl (1 N) to pH=2-3. The residue was detected by LCMS and it was purified by prep-HPLC to obtain P2B-D_peak1 (12.00 mg, 17.61 gmol, yield: 27.22%, 99.324% purity) as white solid which was confirmed by QC-LCMS and ¹H-NMR.

¹H-NMR (400 MHz, D₂O) 7.07-6.95 (m, 10H), 4.02 (d, 2H, J=11.2 Hz), 3.29-3.28 (m, 2H), 3.07-2.99 (m, 6H), 2.87-2.83 (m, 4H), 2.58-2.52 (m, 4H), 2.08-2.06 (m, 2H), 1.91-1.90 (m, 2H), 1.80-1.75 (m, 4H), 1.75-1.66 (m, 4H), 1.53-1.51 (m, 2H).

LCMS: Rt=2.316 min, m/z=631.4 (M+H⁺)

The Peak 3 material from Step 4 was reacted in the same way to prepare P2B-D peak 3.

¹H-NMR (400 MHz, D₂O) 7.08-7.00 (m, 6H), 6.90-6.89 (m, 4H), 3.90 (d, 2H, J=10.8 Hz), 3.30-3.28 (m, 2H), 3.12-2.99 (m, 8H), 2.87-2.82 (m, 4H), 2.57-2.54 (m, 2H), 2.43-2.41 (m, 2H), 2.07-2.05 (m, 2H), 1.87-1.69 (m, 10H).

LCMS: Rt=2.379 min, m/z=631.4 (M+H⁺)

### Example 5: Preparation of 2-[[3-[[carboxy-[(3R)-quinuclidin-3-yl]methyl]carbamoyl]benzoyl]amino]-2-[(3R)-quinuclidin-3-yl]acetic acid (Compound P2B-E)

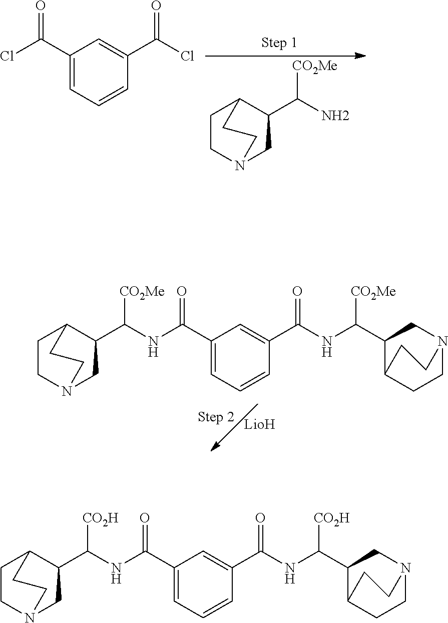 [SMILES: O=C(Cl)c1cccc(C(=O)Cl)c1 ; COC(=O)C(N)[C@H]1CN2CCC1CC2 ; COC(=O)C(NC(=O)c1cccc(C(=O)NC(C(=O)OC)[C@H]2CN3CCC2CC3)c1)[C@H]1CN2CCC1CC2 ; O=C(NC(C(=O)O)[C@H]1CN2CCC1CC2)c1cccc(C(=O)NC(C(=O)O)[C@H]2CN3CCC2CC3)c1]

Step 1: To a solution of methyl 2-amino-2-[(3R)-quinuclidin-3-yl]acetate (100.00 mg, 504.39 gmol, 1.00 eq) in CHCl₃ (4.00 mL) was added benzene-1,3-dicarbonyl chloride (51.20 mg, 252.20 gmol, 0.50 eq) at 30° C. The mixture was stirred for 16 hours at 30° C. The mixture was concentrated under reduced pressure to give a crude product as a yellow solid. Compound methyl 2-[[3-[[(2-methoxy-2-oxo-1-[(3R)-quinuclidin-3-yl]ethyl] carbamoyl]benzoyl] amino]-2-[(3R)-quinuclidin-3-yl]acetate (156.00 mg, crude, HCl) was obtained.

LCMS: RT=0.881, m/z=527.3 (M+H⁺)

Step 2: To a solution of methyl 2-[[3-[[2-methoxy-2-oxo-1-[(3R)-quinuclidin-3-yl]ethyl]carbamoyl]benzoyl]amino]-2-[(3R)-quinuclidin-3-yl]acetate (165.00 mg, 313.31 gmol, 1.00 eq) in THF (4.00 mL) was added LiOH (96.00 mg, 4.01 mmol, 12.79 eq) in H₂O (4.00 mL) at 30° C. The mixture was stirred for 2 hours at 30° C. The mixture was concentrated under reduced pressure to remove THF. To the mixture was added water (10 mL) and HCl (1N) to pH=2. The mixture was concentrated under reduced pressure to give a crude product. The crude product was purified by prep-HPLC to give 2-[[3-[[carboxy-[(3R)-quinuclidin-3-yl]methyl]carbamoyl]benzoyl]amino]-2-[(3R)-quinuclidin-3-yl]acetic acid (Compound P2B-E) (37.20 mg, 63.79 gmol, 40.72% yield, 98% purity, 2HCl) as a white solid, which was analysed by HNMR and LCMS.

¹H NMR (400 MHz, DEUTERIUM OXIDE) δ 8.10-8.01 (m, 1H), 7.88 (dd, J=1.7, 7.8 Hz, 2H), 7.54 (t, J=7.8 Hz, 1H), 4.62 (d, J=11.0 Hz, 2H), 3.59-3.50 (m, 2H), 3.38-3.15 (m, 8H), 3.11-2.99 (m, 2H), 2.71-2.54 (m, 2H), 2.26-2.07 (m, 4H), 2.06-1.78 (m, 6H)

LCMS: RT=5.9, m/z=499.3 (M+H⁺)

### Example 6: Preparation of 2-[[-carboxy-[(3R)-quinuclidin-3-yl]methyl]carbamoylamino]-2-[(3R)-quinuclidin-3-yl]acetic acid (Compound P2B-G)

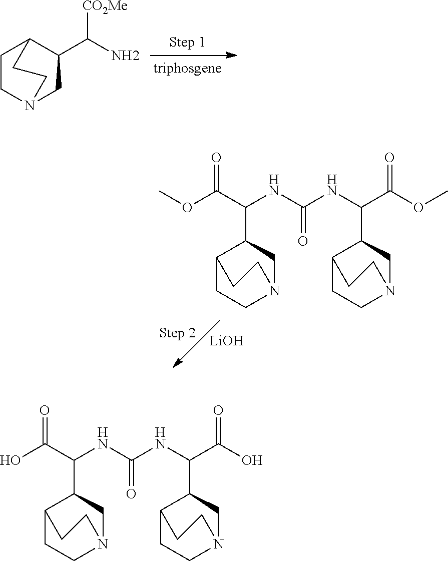

Step 1: To a solution of methyl (2R)-2-amino-2-[(3R)-quinuclidin-3-yl]acetate (100.00 mg, 504.39 gmol, 1.00 eq) and TEA (102.08 mg, 1.01 mmol, 139.83 μL, 2.00 eq) in CHCl₃ (3.00 mL) was added triphosgene (25.45 mg, 85.75 gmol, 0.17 eq) in CHCl₃ (1.00 mL) at 0° C. The mixture was stirred for 20 hours at 30° C. The mixture was concentrated to give a residue. The residue was dissolved into MeOH (5 mL) and purified by prep-HPLC (F to give methyl 2-[[2-methoxy-2-oxo-1-[(3R)-quinuclidin-3-yl]ethyl]carbamoylamino]-2-[(3R)-quinuclidin-3-yl]acetate (150.00 mg, crude) as a white solid, which was analysed by LCMS.

LCMS: RT=6.78, m/z=423.3 (M+H⁺)

Step 2: To a solution of methyl 2-[[2-methoxy-2-oxo-1-[(3R)-quinuclidin-3-yl]ethyl]carbamoylamino]-2-[(3R)-quinuclidin-3-yl]acetate (150.00 mg, 355.01 gmol, 1.00 eq) in THF (2.00 mL) was added LiOH (48.00 mg, 2.00 mmol, 5.65 eq) in H₂O (2.00 mL) at 30° C. The mixture was stirred for 16 hours at 30° C. The mixture was concentrated under reduced pressure to remove THF. To the mixture was added water (10 mL) and HCl (1N) to pH=2. The mixture was concentrated under reduced pressure to give a crude product. The crude product was purified by prep-HPLC to give 2-[[-carboxy-[(3R)-quinuclidin-3-yl]methyl]carbamoylamino]-2-[(3R)-quinuclidin-3-yl]acetic acid (56.00 mg, crude) as a white solid, which was analysed by LCMS and HNMR as UCL_P2B_G.

¹H NMR (400 MHz, DEUTERIUM OXIDE) δ 4.32-4.16 (m, 2H), 3.61-3.38 (m, 2H), 3.32-2.92 (m, 11H), 2.56-2.34 (m, 2H), 2.17-1.77 (m, 2H)

LCMS: m/z=395.2 (M+H⁺)

### Example 7: Preparation of 2-[[4-[[-carboxy-[(3R)-quinuclidin-3-yl]methyl]carbamoyl]cyclohexanecarbonyl]amino]-2-[(3R)-quinuclidin-3-yl]acetic acid (Compound P2B-H)

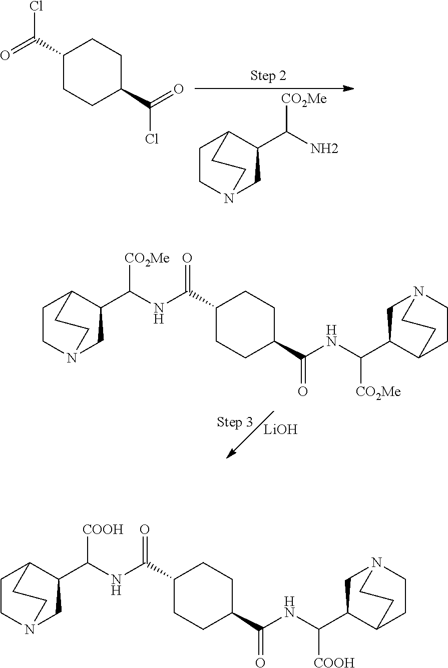 [SMILES: O=C(Cl)[C@H]1CC[C@H](C(=O)Cl)CC1 ; COC(=O)C(N)[C@H]1CN2CCC1CC2 ; COC(=O)C(NC(=O)[C@H]1CC[C@H](C(=O)NC(C(=O)OC)[C@H]2CN3CCC2CC3)CC1)[C@H]1CN2CCC1CC2 ; "LiOH" ; O=C(O)C(NC(=O)[C@H]1CC[C@H](C(=O)NC(C(=O)O)[C@H]2CN3CCC2CC3)CC1)[C@H]1CN2CCC1CC2]

Step 1: To a mixture of cyclohexane-1,4-dicarboxylic acid (1.00 g, 5.81 mmol, 1.00 eq) in DCM (20.00 mL) was added SOCl₂ (2.07 g, 17.43 mmol, 1.26 mL, 3.00 eq) and DMF (4.25 mg, 0.01 eq) at 15° C. The mixture was stirred for 16 hours at 38° C. A sample from reaction mixture was quenched by MeOH. The mixture was concentrated under reduced pressure to give cyclohexane-1,4-dicarbonyl chloride (1.20 g, crude) as a white solid.

LCMS: RT=0.870, m/z=201.1 (M+H⁺)

Step 2: To a solution of methyl 2-amino-2-[(3R)-quinuclidin-3-yl]acetate (95.00 mg, 479.17 gmol, 2.00 eq) in DCM (4.00 mL) was added cyclohexane-1,4-dicarbonyl chloride (50.09 mg, 239.58 gmol, 1.00 eq) at 15° C. The mixture was stirred for 16 hours at 15° C. The mixture was concentrated under reduced pressure to give a crude product. The crude product was purified by prep-HPLC to methyl 2-[[4-[[2-methoxy-2-oxo-1-[(3R)-quinuclidin-3-yl]ethyl]carbamoyl]cyclohexanecarbonyl]amino]-2-[(3R)-quinuclidin-3-yl]acetate (80.00 mg, 148.68 gmol, 62.06% yield, 99% purity) as a white solid, which was analysed by LCMS.

LCMS: RT=0.718, m/z=533.5 (M+H⁺)

Step 3: To a mixture of methyl 2-[[4-[[2-methoxy-2-oxo-1-[(3R)-quinuclidin-3-yl]ethyl]carbamoyl]cyclohexanecarbonyl]amino]-2-[(3R)-quinuclidin-3-yl]acetate (80.00 mg, 150.19 gmol, 1.00 eq) in a mixture of THF (2.00 mL) and H₂O (2.00 mL) was added LiOH (20.21 mg, 844.05 gmol, 5.62 eq) at 15° C. The mixture was stirred for 1 hours at 15° C. The mixture was concentrated under reduced pressure to remove THF. To the mixture was added H₂O (10 mL) and HCl (1N) to pH=2. The mixture was concentrated under reduced pressure to give a crude product. The crude product was purified by Prep-HPLC to give 2-[[4-[[-carboxy-[(3R)-quinuclidin-3-yl]methyl]carbamoyl]cyclohexanecarbonyl]amino]-2-[(3R)-quinuclidin-3-yl]acetic acid (23.60 mg, 43.96 gmol, 29.27% yield, 94% purity) as a white solid, which was analysed by LCMS and HNMR as UCL_P2B_H.

¹H NMR (400 MHz, DEUTERIUM OXIDE) δ 4.38 (d, J=11.0 Hz, 2H), 3.48-3.33 (m, 2H), 3.32-3.10 (m, 8H), 2.92-2.78 (m, 2H), 2.51-2.36 (m, 2H), 2.24 (br s, 2H), 2.16-1.99 (m, 4H), 1.98-1.74 (m, 10H), 1.42-1.26 (m, 4H), 1.17 (t, J=7.3 Hz, 1H)

LCMS: RT=5.44, m/z=505.3 (M+H⁺)

### Example 8: Preparation of Compounds P3A-C and P3A-D

Step 1:

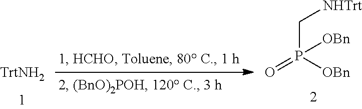

A mixture of triphenylmethanamine (25 g, 96.40 mmol, 1.00 eq) in toluene (250 mL) was combined with HCHO (3.2 g, 106.57 mmol, 2.94 mL, 1.11 eq) and AcOH (1.33 g, 22.17 mmol, 1.27 mL, 0.23 eq) and stirred for 1 hour at 80° C. Then dibenzyl hydrogen phosphite (26 g, 99.15 mmol, 21 mL, 1.03 eq) was added and the reaction mixture was stirred at 120° C. for 3 hours. Then Et₃N (3.61 g, 35.67 mmol, 4.94 mL, 0.37 eq) was added. When the reaction was complete which was detected by LC-MS, the mixture was concentrated in vacuo. The residue was washed with EtOAc and PE (20 mL/100 mL), and filtered, the filter cake was dried to obtain Compound 2 (51 g, crude) as a white solid.

Step 2:

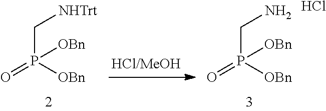

A mixture of Compound 2 (51 g, 95.58 mmol, 1 eq) in HCl/MeOH (250 mL, 2 N) was stirred at 25° C. for 1 hour. When the reaction was complete which was detected by LC-MS, the mixture was concentrated in vacuo to obtain Compound 3 (27 g, crude) as a white solid.

Step 3:

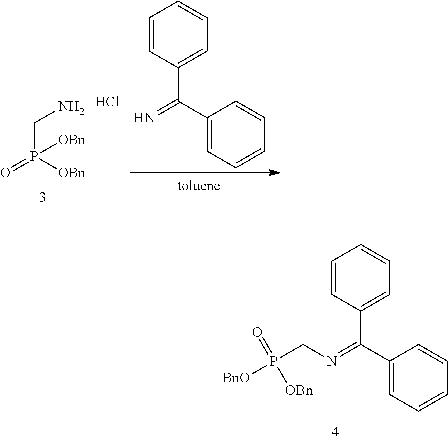 [SMILES: NCP(=O)(OCc1ccccc1)OCc1ccccc1 ; Cl ; N=C(c1ccccc1)c1ccccc1 ; O=P(CN=C(c1ccccc1)c1ccccc1)(OCc1ccccc1)OCc1ccccc1]

To a solution of Compound 3 (36 g, 109.84 mmol, 1.00 eq, HCl) in toluene (200 mL) was added diphenylmethanimine (21.70 g, 119.73 mmol, 20.09 mL, 1.09 eq), the mixture was stirred at 25° C. for 12 hours. When the reaction was complete which was detected on LCMS and TLC (PE:EtOAc=3:1), the mixture was concentrated in vacuo. The residue was purified by column chromatography (SiO₂, Petroleum ether:Ethyl acetate=20:1 to 1:1) to obtain Compound 4 (10 g, 21.95 mmol, 19.99% yield) as a colorless oil.

Step 4:

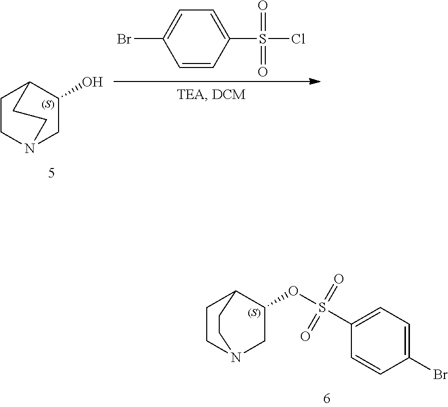

A mixture of Compound 5 (5 g, 39.31 mmol, 1 eq), Et₃N (5.97 g, 58.97 mmol, 8.21 mL, 1.5 eq) in DCM (50 mL) was degassed and purged with N₂ for 3 times, and then the mixture was cooled to 0° C., then to the mixture was added 4-bromobenzenesulfonyl chloride (10.55 g, 41.28 mmol, 1.05 eq), the mixture was stirred at 0° C. for 3 hrs under N₂ atmosphere. When the reaction was complete which was detected by LC-MS, the reaction was quenched by sat. NaHCO₃ (aq., 40 mL) slowly and then extracted with DCM (10 mL*3). The combined organic phase was washed with brine (50 mL), dried over anhydrous Na₂SO₄, filtered and concentrated in vacuo. The residue was purified by column chromatography (SiO₂, Ethyl acetate:Methanol=1:0 to 10:1) to obtain Compound 6 (7.8 g, 22.53 mmol, 57.30% yield) as a white solid which was checked by ¹H NMR.

¹H NMR (400 MHz, CDCl₃) 7.71 (d, 2H, J=8.8 Hz), 7.63 (d, 2H, J=8.8 Hz), 4.59-4.57 (m, 1H), 3.05-3.01 (m, 1H), 2.79-2.57 (m, 5H), 1.94-1.93 (m, 1H), 1.76-1.75 (m, 1H), 1.62-1.61 (m, 1H), 1.39-1.10 (m, 2H).

Step 5:

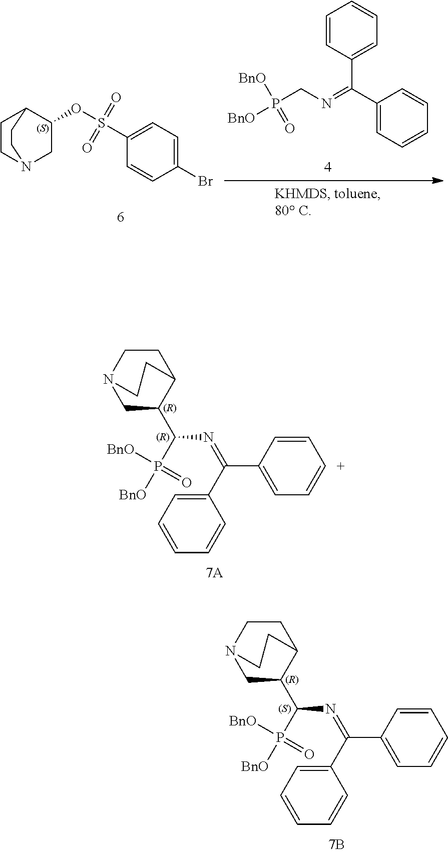

To a solution of Compound 6 (2 g, 5.78 mmol, 1 eq) and Compound 4 (5.26 g, 11.55 mmol, 2 eq) in toluene (40 mL) was added KHMDS (1 M, 11.55 mL, 2 eq) under N₂. The mixture was stirred for 12 hours at 80° C. When the reaction was complete which was detected by LC-MS, the two batches were quenched by sat. NH₄Cl (100 mL, aq.), and then were extracted with EtOAc (50 mL*3). The combined organic phases were washed with brine (150 mL), dried over anhydrous Na₂SO₄, filtered and concentrated in vacuo. The residue was purified by column chromatography (SiO₂, Ethyl acetate:Methanol=1:0 to 0:1) and prep-HPLC (basic condition) (column: Phenomenex luna(2) C18 250*50 10u; mobile phase: [water (10 mM NH₄HCO₃)-ACN]; B %: 19%-49%, 20 min), and get 1 g mixture product. The 1 g mixture product was purified by prep-TLC (SiO₂, Dichloromethane:Methanol=10:1) (with 5% NH₃.H₂O) to obtain Compound 7A (320 mg, crude) as a white solid which was checked by SFC (AD-H_3_10-40-Gradient_2.5_35.met) and ¹H NMR, and Compound 7B (450 mg, crude) as a white solid which was checked by SFC (AD-H_3_10-40-Gradient_2.5_35.met) and ¹H NMR.

¹H NMR (400 MHz, CDCl₃) 7.61-7.27 (m, 20H), 5.11-5.06 (m, 1H), 4.97-4.92 (m, 1H), 4.54-4.51 (m, 1H), 4.20-4.17 (m, 1H), 3.87-3.52 (m, 6H), 3.22-3.20 (m, 1H), 2.66-2.65 (m, 2H), 1.91-1.79 (m, 4H), 1.51-1.45 (m, 1H).

¹H NMR (400 MHz, CDCl₃) 7.33-7.01 (m, 20H), 4.70-4.65 (m, 2H), 4.50-4.47 (m, 1H), 4.34-4.33 (m, 1H), 4.01-3.74 (m, 6H), 3.52-3.51 (m, 1H), 3.39-3.38 (m, 1H), 3.14-3.13 (m, 1H), 2.89-2.87 (m, 2H), 1.81-1.80 (m, 1H), 1.60-1.56 (m, 2H), 1.35-1.34 (m, 1H).

Step 6:

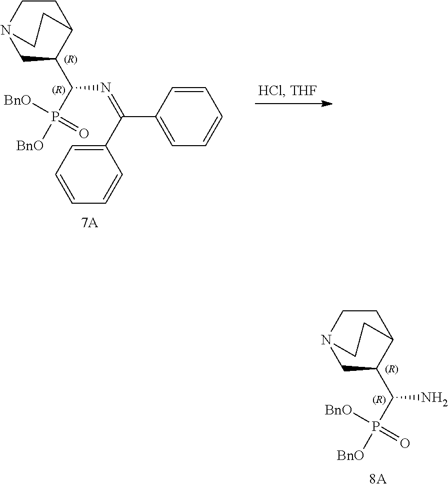

To a solution of Compound 7A (320.00 mg, 566.72 gmol, 1 eq) in THF (5 mL) was added HCl (502.62 mg, 5.10 mmol, 492.76 μL, 37% purity, 9 eq) at 0° C. The mixture was stirred for 1 hour at 0° C. and 11 hours at 25° C. LCMS showed Compound 7A was consumed completely and one main peak with desired MS was detected. The mixture was dissolved in H₂O (10 mL), then washed with TBME (5 mL*3). Then the aqueous phase was concentrated in vacuum to obtain Compound 8A (250 mg, crude, 2HCl) as a yellow oil which was confirmed by ¹H NMR and used directly in the next step without further purification.

¹H NMR (400 MHz, CD₃OD) 7.52-7.32 (m, 10H), 5.07-5.04 (m, 1H), 4.48-4.35 (m, 2H), 3.74-3.59 (m, 1H), 3.54-3.39 (m, 5H), 3.31-3.20 (m, 2H), 2.68-2.54 (m, 1H), 2.22-2.13 (m, 3H), 2.00-1.92 (m, 2H).

Step 7:

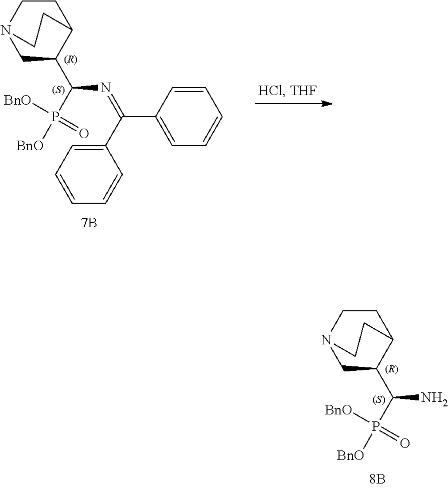

To a solution of Compound 7B (400 mg, 708.40 gmol, 1 eq) in THF (5 mL) was added HCl (349.03 mg, 3.54 mmol, 342.19 μL, 37% purity, 5 eq) at 0° C. The mixture was stirred for 1 hour at 0° C. and 11 hours at 25° C. LCMS showed Compound 7B was consumed completely and one main peak with desired MS was detected. The mixture was dissolved in H₂O (10 mL), then washed with TBME (5 mL*3). Then the aqueous phase was concentrated in vacuum and then by lyophilization to obtain Compound 8B (320 mg, crude, 2HCl) as a white solid which was checked by ¹H NMR and used directly in the next step.

¹H-NMR (400 MHz, CD₃OD) 7.56-7.34 (m, 10H), 5.12-5.06 (m, 1H), 4.52-4.46 (m, 2H), 3.72-3.30 (m, 7H), 3.21-3.20 (m, 1H), 2.66-2.60 (m, 1H), 2.14-1.85 (m, 5H).

Step 8:

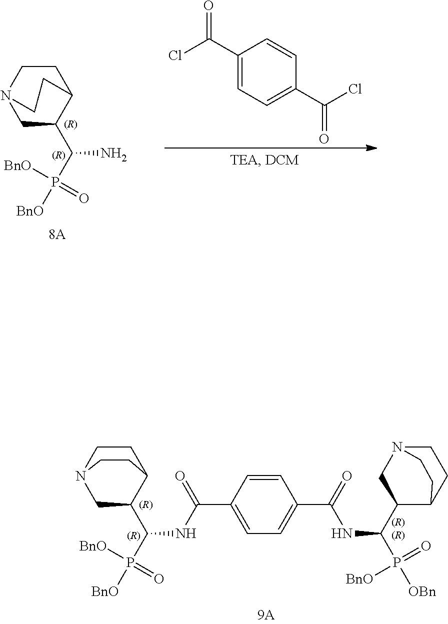

To the solution of Compound 8A (160.00 mg, 338.00 gmol, 1 eq, 2HCl) in DCM (4 mL) was added Et₃N (171.01 mg, 1.69 mmol, 235.23 μL, 5.00 eq) at 0° C., it was stirred at 0° C. for 10 mins, then the solution of benzene-1,4-dicarbonyl chloride (30.88 mg, 152.10 gmol, 0.45 eq) in DCM (1 mL) was added at 0° C., after addition, the resulting mixture was stirred at 25° C. under the protection of N₂ for 12 hrs. LCMS showed some Compound 8A was remained, and the ms of 548 was detected, so T₃P (215.09 mg, 338.00 gmol, 201.02 μL, 50% purity, 1 eq) was added, and the resulting mixture was stirred at 25° C. for 1 hr. The mixture was concentrated under vacuum to give a crude product. The crude product was purified by prep-HPLC (TFA) to obtain Compound 9A (18.00 mg, 17.22 gmol, 5.10% yield, TFA) as colorless oil which was used directly in the next step.

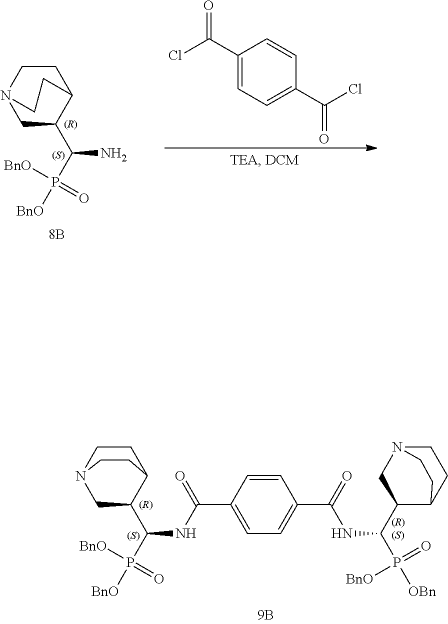

To the solution of Compound 8B (80.00 mg, 169.00 gmol, 1 eq, 2HCl) in DCM (6 mL) was added Et₃N (85.51 mg, 845.00 gmol, 117.61 μL, 5.00 eq) at 0° C., it was stirred at 0° C. for 10 mins, then the solution of benzene-1,4-dicarbonyl chloride (15.44 mg, 76.05 gmol, 0.45 eq) in DCM (1 mL) was added at 0° C., after addition, the resulting mixture was stirred at 25° C. under the protection of N₂ for 12 hrs. LCMS showed some Compound 8B was remained, so more Et₃N (34.20 mg, 338.00 gmol, 47.05 μL, 2.00 eq) was added, and the resulting mixture was stirred at 25° C. for another 1 hr. The mixture was concentrated under vacuum to give a crude product. The crude product was purified by prep-HPLC (TFA) to obtain Compound 9B (30.00 mg, 28.71 gmol, 16.99% yield, TFA) as colorless oil which was used directly in the next step.

Step 9:

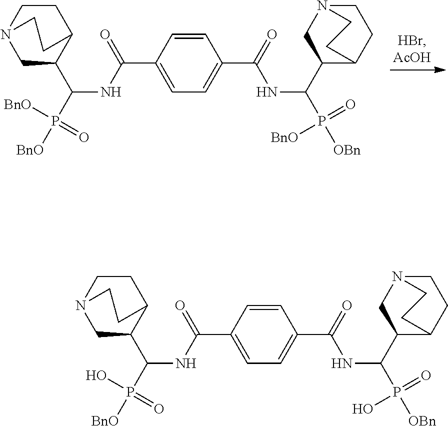 [SMILES: O=C(NC([C@H]1CN2CCC1CC2)P(=O)(OCc1ccccc1)OCc1ccccc1)c1ccc(C(=O)NC([C@H]2CN3CCC2CC3)P(=O)(OCc2ccccc2)OCc2ccccc2)cc1 ; O=C(NC([C@H]1CN2CCC1CC2)P(=O)(O)OCc1ccccc1)c1ccc(C(=O)NC([C@H]2CN3CCC2CC3)P(=O)(O)OCc2ccccc2)cc1]

To the solution of Compound 9B (30.00 mg, 28.71 gmol, 1 eq, TFA) in AcOH (1 mL) was added HBr (1 mL) (40% in water), the resulting mixture was stirred at 25° C. for 12 hrs. When the reaction was complete which was detected by LCMS, the mixture was concentrated under vacuum to give a crude product. The crude product was purified by prep-HPLC (HCl) to obtain P3A-C (4.90 mg, 5.87 gmol, 20.43% yield, 94.229% purity, HCl) as yellow solid which was confirmed by ¹H-NMR, PNMR and special LCMS.

(It is possible that some of the P3A-C could also arise directly from the presence of monobenzyl precursor present as a by-product in the product of Step 8.)

¹H-NMR (400 MHz, CD₃OD) 7.84 (s, 4H), 7.44-7.30 (m, 10H), 7.47-7.41 (m, 1H), 4.35-4.32 (m, 4H), 3.61-3.60 (m, 1H), 3.42-3.38 (m, 9H), 3.03-3.00 (m, 2H), 2.68-2.65 (m, 4H), 2.19-2.18 (m, 2H), 2.00-1.91 (m, 4H), 1.86-1.84 (m, 2H).

LCMS: Rt=1.480 min, m/z=751.4 (M+H⁺)

To the solution of Compound 9A (18.00 mg, 17.22 gmol, 1 eq, TFA) in AcOH (2 mL) was added HBr (2 mL) (40% in water), the resulting mixture was stirred at 25° C. for 12 hrs. When the reaction was complete which was detected by LCMS, the mixture was concentrated by N₂ stream to give a crude product. The crude product was purified by prep-HPLC (HCl) to obtain P3A-D (4.10 mg, 5.08 gmol, 29.51% yield, 97.602% purity, HCl) as yellow solid which was confirmed by, ¹H-NMR, PNMR and QC-LCMS.

¹H-NMR (400 MHz, CD₃OD) 7.95 (s, 4H), 7.57-7.55 (m, 10H), 7.62-7.58 (m, 2H), 4.51-4.45 (m, 4H), 3.86-3.83 (m, 2H), 3.57-3.33 (m, 10H), 2.72-2.69 (m, 2H), 2.21-2.20 (m, 4H), 2.15-2.00 (m, 4H), 1.88-1.85 (m, 2H).

LCMS: Rt=1.717 min, m/z=751.4 (M+H⁺)

### Example 9: Preparation of 2-[[4-[[-carboxy-[(3R)-1-methylpyrrolidin-3-yl]methyl]carbamoyl]benzoyl]amino]-2-[(3R)-1-methylpyrrolidin-3-yl]acetic acid (Compound PSA-B)

Step 1: To a mixture of methyl (2S)-2-amino-2-[(3R)-1-methylpyrrolidin-3-yl]acetate (150.00 mg, 718.77 gmol, 1.00 eq, HCl) in MeOH (10.00 mL) was added Ambersep 900 (OH) (2.00 g, 718.77 gmol, 1.00 eq) at 15° C. The mixture was stirred for 1 hour at 15° C.

LCMS showed desired product was detected.

The mixture was filtered and filtrate was concentrated under reduced pressure to give a residue. To the residue was added CHCl₃ (20 mL) and filtered. The filtrate was concentrated under reduced pressure to give methyl (2S)-2-amino-2-[(3R)-1-methylpyrrolidin-3-yl]acetate (80.00 mg, crude) as a yellow oil.

Step 2:

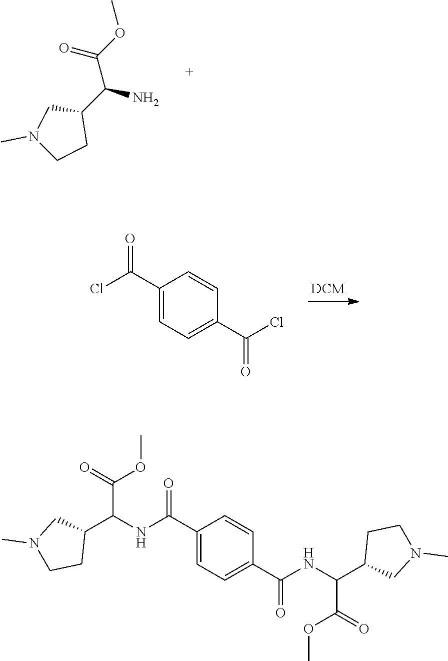

To a solution of methyl (2S)-2-amino-2-[(3R)-1-methylpyrrolidin-3-yl]acetate (80.00 mg, 464.50 gmol, 1.00 eq) in DCM (4.00 mL) was added benzene-1,4-dicarbonyl chloride (47.15 mg, 232.25 gmol, 0.50 eq) at 10° C. The mixture was stirred for 16 hours at 10° C.

LCMS showed desired product was detected.

The mixture was concentrated under reduced pressure to give methyl (2S)-2-[[4-[[(1S)-2-methoxy-1-[(3R)-1-methylpyrrolidin-3-yl]-2-oxo-ethyl]carbamoyl]benzoyl]amino]-2-[(3R)-1-methylpyrrolidin-3-yl]acetate (120.00 mg, crude) as a yellow solid.

Step 3:

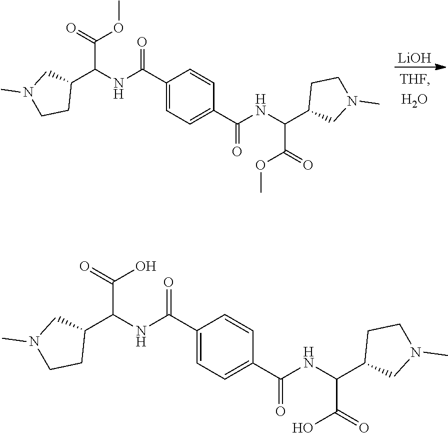

To a solution of methyl (2S)-2-[[4-[[(1S)-2-methoxy-1-[(3R)-1-methylpyrrolidin-3-yl]-2-oxo-ethyl]carbamoyl]benzoyl]amino]-2-[(3R)-1-methylpyrrolidin-3-yl]acetate (120.00 mg, 252.87 gmol, 1.00 eq) in a mixture of THF (2.00 mL) and H2O (2.00 mL) at 10° C. The mixture was stirred for 16 hours at 10° C. To the mixture was added LiOH (23.98 mg, 1.00 mmol, 3.96 eq) at 10° C. The mixture was stirred for 6 hrs at 10° C.

LCMS showed desired product was detected.

To the mixture was added HCl (1N) to pH=2. The mixture was concentrated under reduced pressure to give a crude product.

The crude product was purified by Prep-HPLC to give 2-[[4-[[-carboxy-[(3R)-1-methylpyrrolidin-3-yl]methyl]carbamoyl]benzoyl]amino]-2-[(3R)-1-methylpyrrolidin-3-yl]acetic acid (32.40 mg, 71.84 gmol, 28.41% yield, 99% purity) as a white solid, which was analysed by LCMS and HNMR.

### Example 10: Preparation of Compounds PK-023 and PK-025 to PK-028

The above compounds were prepared according to the following general reaction scheme:

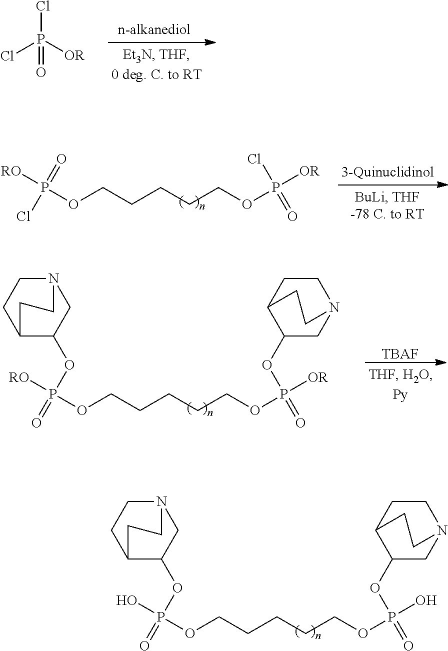

wherein the protecting group R is 4-chlorophenyl, and n=1 to 5.

Specifically, compound Pk-023 (n=4 in the above schemes) was prepared as follows according to the above general scheme (but with a different base for deprotection of the phosphate group in the final step):

Step 1: A solution of phenyldichlorophosphate (5.0 g, 23.6 mmol) in tetrahydrofuran (50 ml) was cooled to 0° C. and a solution of 1,8-octanediol (2.0 g, 11.8 mmol, 0.5 eq) and triethylamine (3.9 ml, 1.2 eq) in tetrahydrofuran (100 ml) added dropwise. The resultant mixture was stirred to room temperature overnight, after which time analysis by ³¹P NMR showed a new product had formed. The reaction was filtered, a small portion was concentrated for analysis and the remainder stored under Argon for use in subsequent reactions.

³¹P NMR: δ 0.36 ppm (major signal)

¹H NMR: corresponds to desired product (˜85-90%)

Step 2: A solution of (S)-quinuclidinol (1.27 g, 10 mmol) in tetrahydrofuran (20 ml) was stirred under argon and cooled to −78° C. A solution of n-butyllithium (7 ml, 1.7 M in hexanes, 11.9 mmol) was added dropwise and the resultant solution stirred to ambient temperature over 1 hour. After cooling to 0° C., a solution of octane-1,8-diyl diphenyl bis(phosphorochloridate) in tetrahydrofuran (35 ml, ca. 2.75 mmol) was added and the reaction stirred to ambient temperature over three hours. Analysis by 31P NMR showed the disappearance of the starting material and formation of a new product. The reaction mixture was diluted with ethyl acetate, washed with water, dried and concentrated. The residue thus obtained (2.1 g) was washed with diethyl ether, re-concentrated to remove residual solvent and used without further purification in the subsequent reaction.

Step 3: To an aqueous solution of barium hydroxide. 8H₂O (0.1M, 27 ml, 2.7 mmol) heated at 95° C. was added 8-(((phenyloxy)(quinuclidin-3-yloxy)phosphoryl)oxy)octyl phenyl quinuclidin-3-yl phosphate (200 mg). After heating overnight, the reaction was allowed to cool and an aqueous solution of ammonium sulfate (530 mg in 27 ml) added. After stirring for fifteen minutes, the mixture was filtered, and the filtrate concentrated in vacuo. The residue was combined with those obtained from three similar runs (1×200 mg scale & 2×150 mg scale) and purified by PEAX column eluted first with methanol, then 1% HCl in methanol. The acidic fractions were concentrated, and the residue obtained further purified on a mass directed auto-purification system (MDAP). Appropriate fractions were concentrated to give the desired product−yield=8.8 mg.

¹H NMR (400 MHz, CDOD) δ 8.36 (OH, 2H), 4.54 (m, 2H), 3.86 (m, 4H), 3.60 (m, 2H), 3.35-3.15 (m, 10H), 2.35 (m, 2H), 2.25 (m, 2H), 2.01 (m, 2H), 1.90-1.75 (m, 4H), 1.65-1.55 (m, 4H), 1.45-1.30 (m, 8H).

¹³C NMR (300 MHz, CDOD) δ 67.61, 64.43, 54.56, 46.50, 45.00, 30.45, 28.79, 25.41, 19.82, 16.22

³¹P NMR (300 MHz, CDOD) δ 0.48

HPLC (Acid method, ELS): 2 peaks (ratio 75.4:24.6) both showing [M−H]-=523.31 (ES⁻).

### III. Properties of Ligand Compounds

### 1. Results—Immobilised Phosphocholine Plate Assay

The calcium dependent binding of CRP to phosphocholine covalently immobilised on 96-well immunoassay plates is reversibly inhibited by free soluble ligand compounds that occupy the ligand binding site. The bis-phosphocholine ligand BPC8 described in WO03/097104 (see above) was a very effective inhibitor of ¹²⁵I-CRP binding to immobilised phosphocholine, with an IC₅₀ of 2.2 μM—five times better than that of free phosphocholine itself.

The bivalent alkyl bis(phosphoquinuclidines), including the C8 moiety Pk-023, of the present disclosure (see above for formula), resemble BPC8 but with the choline replaced by the more drug-like quinuclidine. In the PC-plate assay, Pk-023 was an even more potent inhibitor of CRP binding than BPC8 with an IC₅₀ of 0.15 μM. A few other alkyl bis(phosphoquinuclidines) were tested, and were similarly effective, as shown in Table 1 below. Clearly, replacement of choline by quinuclidine generated a superior CRP ligand.

TABLE 1
IC₅₀ values for inhibition of ¹²⁵I-CRP binding to immobilised
phosphocholine by bivalent ligands.

| Compound | IC₅₀ (μM) | Structure |
|---|---|---|
| Pk-023 | 0.15 | Octane-1,8-diyl di(S)-quinuclidin-3-yl) |
| | | bis(hydrogen phosphate |
| Pk-025 | 0.28 | Hexane-1,6-diyl di(S)-quinuclidin-3-yl) |
| | | bis(hydrogen phosphate |
| Pk-026 | 0.18 | Heptane-1,7-diyl di(S)-quinuclidin-3-yl) |
| | | bis(hydrogen phosphate |
| Pk-027 | 0.18 | Decane-1,10-diyl di(S)-quinuclidin-3-yl) |
| | | bis(hydrogen phosphate |
| Pk-028 | 0.16 | Nonane-1,9-diyl di(S)-quinuclidin-3-yl) |
| | | bis(hydrogen phosphate |
| BPC8 | 2.2 | 1,8-bis(phosphocholine)-octane |

The PC-plate assay is a low-throughput, time-consuming, manual assay that requires a constant supply of freshly radiolabelled CRP. The Roche immunoturbidimetric CRP assay on the automated COBAS MIRA instrument is more suitable for screening purposes.

### 2. Results—Roche Immunoturbidimetric Assay

The immunoturbidimetric CRP assay on the Roche COBAS MIRA Plus autoanalyser, utilises two different sized microparticles that are covalently coupled with two different monoclonal antibodies, which recognise different CRP epitopes. The assay was developed by Roche for clinical measurement of CRP serum and plasma with high sensitivity and a very wide dynamic range. Serendipitously, one of the two antibodies binds to an epitope present on the ligand binding B-face of CRP. Thus, when the binding pocket is occupied by ligand and, especially, when the B-face is occluded by the B-face to B-face interaction of two native CRP pentamers produced by cross linking via our unique bivalent ligands, the assay fails to detect the CRP. In contrast, the CRP is all detected and assayed normally by other assays that disrupt the crosslinking and/or use antibodies which recognise different, non-occluded, epitopes. Inhibition of CRP recognition in the Roche assay is thus a convenient tool to monitor the efficacy and potency of complex formation between our unique ligands and CRP.

BPC8 produced dose-dependent inhibition of Roche CRP recognition, with a 2.5-fold excess of ligand over CRP pentamer causing 50% reduction of immunoreactivity. The reproducibility of results, determined in 12 assays, was excellent. The mean and SD (range) of IC₅₀ concentrations were 1.88, 0.51 (0.83-2.99) μM. Other bis(phosphocholines) with varying alkyl chain linkers also reduced Roche CRP immunoreactivity, but none were as good as BPC8. The C8 bis(phosphoquinuclidine) Pk-023 was about 3-fold more potent than BPC8 with a mean (SD) IC₅₀ of 0.60 (0.06) μM. There was no evidence that a large excess of ligand, with the resultant saturation of the PC binding site on every CRP pentamer, could inhibit cross linking of pairs of CRP molecules. At the maximum concentration tested, >650-fold molar excess of compound over CRP pentamer, all of the CRP was still cross linked in pairs of pentamers. This important observation demonstrates that even the highest conceivable dosing with our bivalent cross linking ligands does not abrogate their desired therapeutic effect of inhibiting the pathogenic binding of CRP to autologous cellular ligands in vivo.

When screened in the Roche assay (Table 2), several bis(quinuclidine)-carboxylates according to the present invention were more effective inhibitors than BPC8. These included P1d-0, P1d-2, P1d-4, P2B-B, P2B-D peak 1 and P2B-H. Of the quinuclidine phosphonate compounds, free phosphonic acid and its di-benzyl ester failed to inhibit the Roche CRP assay, but the bismonobenzyl derivative (P3A-C) was one of the most effective compounds examined (IC₅₀<1 μM)

TABLE 2
Inhibition of Roche CRP immunoreactivity by bivalent compounds, ranked by IC₅₀.

| Compound | IC₅₀ (μM), SD | Structure |
|---|---|---|
| P1d-1 | >400 | 2-[(3R)-1-azabicyclo[2.2.2]octan-3-yl]-2-(2-{[[(3R)-1-azabicyclo[2.2.2]octan-3- |
| | | yl](carboxy)methyl]carbamoyl}acetamido)acetic acid |
| P1d-3 | >400 | 2-[(3R)-1-azabicyclo[2.2.2]octan-3-yl]-2-(4-{[[(3R)-1-azabicyclo[2.2.2]octan-3- |
| | | yl](carboxy)methyl]carbamoyl}butanamido)acetic acid |
| P1d-5 | >400 | 2-[(3R)-1-azabicyclo[2.2.2]octan-3-yl]-2-(6-{[[(3R)-1-azabicyclo[2.2.2]octan-3- |
| | | yl](carboxy)methyl]carbamoyl}hexanamido)acetic acid |
| P1d-7 | >400 | 2-[(3R)-1-azabicyclo[2.2.2]octan-3-yl]-2-(8-{[[(3R)-1-azabicyclo[2.2.2]octan-3- |
| | | yl](carboxy)methyl]carbamoyl}octanamido)acetic acid |
| Dm-BPC8 | 127.0 | bis(2-(dimethylamino)ethyl) octane-1,8-diyl bis(hydrogen phosphate). |
| | | (BPC8 analogue with replacement of choline by dimethlyamino.) |
| P2B-G | 26.17 | 2-[(3R)-1-azabicyclo[2.2.2]octan-3-yl]-2-[({[(3R)-1-azabicyclo[2.2.2]octan-3- |
| | | yl](carboxy)methyl}carbamoyl)amino]acetic acid |
| P1d-8 | 18.8, 15.3 | 2-[(3R)-1-azabicyclo[2.2.2]octan-3-yl]-2-(9-{[[(3R)-1-azabicyclo[2.2.2]octan-3- |
| | | yl](carboxy)methyl]carbamoyl}nonamido)acetic acid |
| P1d-6 | 9.92 | 2-[(3R)-1-azabicyclo[2.2.2]octan-3-yl]-2-[7-({[(3R)-1-azabicyclo[2.2.2]octan-3- |
| | | yl](carboxy)methyl}carbamoyl)heptanamido]acetic acid |
| BPC6 (crude) | 4.70 | {[6-({hydroxy[2-(trimethylazaniumyl)ethoxy]phosphoryl}oxy)hexyl]oxy}[2- |
| | | (trimethylazaniumyl)ethoxy]phosphinic acid. Hexyl-1,6 bisphosphocholine. |
| P2B-E | 4.56, 1.99 | 2-[(3R)-1-azabicyclo[2.2.2]octan-3-yl]-2-{[3-({[(3R)-1-azabicyclo[2.2.2]octan-3- |
| | | yl](carboxy)methyl}carbamoyl)phenyl]formamido}acetic acid |
| P2B-C | 2.69 | 2-[(3R)-1-azabicyclo[2.2.2]octan-3-yl]-2-[({4-[({[(3R)-1-azabicyclo[2.2.2]octan-3- |
| | | yl](carboxy)methyl}amino)methyl]phenyl}methyl)amino]acetic acid |
| | | P2B_B analogue with a 1,4-CH₂.Ph.CH₂-linker. |
| BPC8 | 1.88, 0.51 | {[8-({hydroxy[2-(trimethylazaniumyl)ethoxy]phosphoryl}oxy)octyl]oxy} |
| | (n = 12) | [2-(trimethylazaniumyl)ethoxy]phosphinic acid. |
| P1d-4 | 1.61, 0.09 | 2-[(3R)-1-azabicyclo[2.2.2]octan-3-yl]-2-(8-{[[(3R)-1-azabicyclo[2.2.2]octan-3- |
| | (n = 3) | yl](carboxy)methyl]carbamoyl}pentamido)acetic acid |
| P1d-4A | 1.43 | (3R)-3-{carboxy[5-({carboxy[(3R)-1-methyl-1-azabicyclo[2.2.2]octan-1-ium-3- |
| | | yl]methyl}carbamoyl)pentanamido]methyl}-1-methyl-1-azabicyclo[2.2.2]octan-1-ium |
| P1d-2 | 1.23, 0.06 | 2-[(3R)-1-azabicyclo[2.2.2]octan-3-yl]-2-[3-({[(3R)-1-azabicyclo[2.2.2]octan-3- |
| | (n = 3) | yl](carboxy)methyl}carbamoyl)propanamido]acetic acid |
| Pk-025 | 1.08 | [(3R)-1-azabicyclo[2.2.2]octan-3-yloxy]({[6-({[(3R)-1-azabicyclo[2.2.2]octan-3- |
| | | yloxy](hydroxy{phosphoryl}oxy)hexyl]oxy})phosphinic acid |
| P1d-0 | 0.97 | 2-[(3R)-1-azabicyclo[2.2.2]octan-3-yl]-2-[({[(3R)-1-azabicyclo[2.2.2]octan-3- |
| | | yl](carboxy)methyl}carbamoyl)formamido]acetic acid |
| P3A-C | 0.91 | {[(3R)-1-azabicyclo[2.2.2]octan-3-yl]({[4-({[(3R)-1-azabicyclo[2.2.2]octan-3- |
| | | yl][(benzyloxy)(hydroxy)phosphoryl]methyl}carbamoyl)phenyl]formamido})methyl} |
| | | (benzyloxyphosphinic acid |
| Pk-032 | 0.72 | di((S)-1-azabicyclo[2.2.2]octan-3-yl) (E)-2,2,7,7-tetramethyloct-4-ene-1,8-diyl |
| | | bis(hydrogen phosphate). Unsaturated tetramethyl linker analogue of Pk-023. |
| P2B-D peak1 | 0.66 | 2-[(3R)-1-azabicyclo[2.2.2]octan-3-yl]-2-(5-{[[(3R)-1-azabicyclo[2.2.2]octan-3- |
| | | yl](carboxy)methyl]carbamoyl}-3,4-diphenylpentanamido)acetic acid. |
| | | (Stereochemistry not characterised.) |
| P2B-B | 0.64 ± 0.17 | 2-[(3R)-1-azabicyclo[2.2.2]octan-3-yl]-2-{[4-({[(3R)-1-azabicyclo[2.2.2]octan-3- |
| | (n = 3) | yl](carboxy)methyl}carbamoyl)phenyl]formamido}acetic acid |
| Pk-023 | 0.60, 0.06 | [(3S)-1-azabicyclo[2.2.2]octan-3-yloxy]({[8-({[(3S)-1-azabicyclo[2.2.2]octan-3- |
| | (n = 5) | yloxy](hydroxy)phosphoryl}oxy)octyl]oxy})phosphinic acid |
| | | (Highly purified gold standard.) |
| P2B-H | 0.60 | 2-[(3R)-1-azabicyclo[2.2.2]octan-3-yl]-2-[(4-{[[(3R)-1-azabicyclo[2.2.2]octan-3- |
| | | yl](carboxy)methyl]amino}cyclohexyl)amino]acetic acid |
| Pk-033 | 0.59 | [(3S)-1-azabicyclo[2.2.2]octan-3-yloxy]({[8-({[(3S)-1-azabicyclo[2.2.2]octan-3- |
| | | yloxy](hydroxy)phosphoryl}oxy)-2,2,7,7-tetramethyloctyl]oxy})phosphinic acid |

(In Table 2: The stereochemistry of the P1-P3 series is assigned as (3R)-quinuclidinyl, with Ca undefined. 1-azabicyclo[2.2.2]octan-3-yl is the 3-linked quinuclidine group, quinuclidin-3-yl.)

Interestingly, the odd numbered alkane chain linkers in the P1D series (C1-C7; P1d-1, P1d-3, P1d-5, P1d-7) were poor inhibitors with IC₅₀ values >400 μM, whereas the even numbered alkane series (C0-C8; P1d-0, P1d-2, P1d-4, P1d-6 [Pk-025], P1d-8 [Pk-023]) were excellent. P1d-2, P1d-4 and, surprisingly, P1d-0, were also very good (IC₅₀<2 μM). The bis(phosphoquinuclidines) Pk-023 and two alkane linker analogues Pk-032 and Pk-033 (2,2,7,7 tetramethyl C8:2 and 2,2,7,7 tetramethyl C8) exhibited the lowest IC₅₀ values, although these are so close to the LOD of the assay that the actual values may not be robust

### 3. Results—Size Exclusion Chromatography

Size exclusion chromatography on a Superdex 200 column equilibrated and eluted with TC buffer pH 8.0, and calibrated with standard globular marker proteins and with human CRP, the native CRP pentamer (Mᵣ 115 kDa) eluted at 13.9 ml (range 13.7-14.0 ml, n=10).

Following pre-incubation with either BPC8 or Pk-023, CRP eluted from Superdex 200 at 12.6 ml, indicating that pairs of pentameric CRP molecules were in stable cross-linked complexes that remained intact after removal of all free ligand by gel filtration. Cross linking was dose-dependent for CRP/Pk-023 interaction. Here, a ˜1.6-fold excess of ligand over CRP pentamer was sufficient to convert 50% of pentamer, with a five-fold excess resulting in almost complete cross linking. Incubation with a 50-fold excess of ligand did not affect the amount of cross linking.

When the fraction containing the CRP/Pk-023 complex was re-chromatographed, the complex remained intact, eluting at the same volume as before. The complex was also stable after overnight storage in TC buffer at room temperature.

The other quinuclidine and phosphonate compounds of interest also all generated CRP pair complexes isolated by SEC and stable to rechromatography (Table 3). In the case of P2B-B, a ˜1.8-fold ratio of ligand:CRP resulted in 50% cross linking. There was a small increase in retention volume for the re-chromatographed complex together with a slight broadening of the peak; this suggests that the complex is at the limit of its stability at this dilution and in the absence of free ligands, and is beginning to decompose on the column. Consistent with the steeper Hill slope of the inhibition curves obtained in the Roche CRP assay for Pk-023 and P2B-B compared to BPC8, the CRP/BPC8 complex was not stable to rechromatography and dissociated to the native single pentamer.

TABLE 3
EC₅₀ of ligand: CRP ratios for bivalent compounds cross linking
CRP pentamers determined by size exclusion chromatography.

| | Ligand/CRP | |
|---|---|---|
| Ligand | ratio: EC50 | Stable to rechromatography |
| BPC8 | 2.5 | No |
| Pk-023 | 1.6 | Yes |
| P1d-2 | 3.2 | Yes |
| P1d-4 | 2.4 | Partial |
| P2B-B | 1.8 | Yes |
| P2B-D pk1 | 2.1 | Yes |
| P2B-H | 1.6 | Yes |

### 4. Results—Stability in Mouse Plasma and Whole Blood

Pre-formed complexes of CRP/Pk-023 (with 5-fold molar excess of ligand over CRP pentamer), or CRP alone, were co-incubated, unstirred, in TC buffer (control), fresh mouse heparinised plasma or fresh heparinised whole mouse blood at 37° C. for up to 3 hours. CRP concentrations in plasma were then measured by Roche immunoassay. The complexes were stable throughout the experiment with low Roche CRP concentrations in the presence of ligand. On addition of EDTA to dissociate CRP ligand binding, Roche CRP concentrations in all CRP/Pk-023 samples recovered to ˜70% of control values showing that the protein was still present and immunoreactive.

### 5. Results—Stability to Shear Forces

To determine whether shear forces might have an effect on stability, pre-formed CRP/Pk-023 complexes prepared with either a 2.5- or 5-fold molar excess of ligand were added to heparinised mouse plasma and stirred rapidly at room temperature for 3 h. Again, the complexes were stable throughout the experiment as demonstrated by the inhibition of Roche CRP reactivity.

### 6. Results—Stability to Dialysis

CRP/Pk-023 complexes formed at 5-fold ligand excess were stable during exhaustive dialysis against TC buffer for up to 96 h at room temperature, as shown by the Roche CRP assay values. After 30 h, ˜8% of the complex had dissociated, with a further 2.5% dissociation at 96 h. Similarly, CRP/Pk-023 complexes pre-formed in serum were stable during dialysis against TC buffer for 24 h at room temperature with shear forces produced by a magnetic stir bar inserted into the dialysis tubing. ¹²⁵I-CRP was included to correct for dilution account for any effects of dilution. The Roche CRP value did not change and addition of EDTA to the 24 h samples resulted in full recovery of Roche CRP reactivity.

### 7. Results—X-ray Crystallography

A. Bis(phosphocholine)-alkanes

X-ray crystal structures were determined for the C5-C9 series of bis(phosphocholine)-alkane (BPC) compounds in complex with CRP using the CRP-phosphocholine complex (pdb code B109) as the search model for phase determination by molecular replacement. The crystal quality was variable across the series, providing data between 2.5 and 3.5 Å resolution.

All structure solutions showed CRP pentamers positioned in a B-face to B-face fashion with clear electron density for the phosphocholine head group and more diffuse density for the linker atoms. As the linker length increased, the relative rotations of the constituent pentamers of the cross linked CRP decamers about their common five-fold axis gradually increased from 14 to 22°. Changes in pentamer packing occur providing orthorhombic crystals for C5,C6: monoclinic for C7: hexagonal for C8,C9. The rotations reflect the exit bond vector of the phosphate-ether that in turn derive from the orientation of the phosphate oxygen ligands to the protein bound calcium ions.

It is likely that the initial product of interaction would be a rather open decamer to enable access to all sites, but that the full complex condenses with inclined ligands both directing and limiting the rotations. Of these compounds, the C8 ligand, BPC8, produced superior results in binding and inhibition studies. The protein-protein interactions in the structure of the BPC8-induced CRP decamer interface, are not extensive and there are quite a few water molecules present. However, an interaction does occur in the region of residues 68-71, a $-turn protruding on the B-face and quite close to the ligand binding site. As this turn is close to the 5-fold axis of the decamer the rotations have a subtle, pivot-like, impact on the contact site.

B. Bis(quinuclidine)-phosphates

Crystallisation of these compounds with CRP was difficult. Co-crystals were obtained with Pk-012, Pk-023, Pk-024 and Pk-029. In all cases the data resolution was limited to between 3.5-4.0 Å. In some cases, the unit cell volume was very big, for example an axis of ˜500 Å was obtained for Pk-023. This created problems in structure solution. In all cases the electron density for the ligand was essentially a “blob”. However, the blob was in the vicinity of the expected ligand binding site.

C. Bis(quinuclidine) amino-carboxylates

Crystal structures were determined for all members of this family that showed inhibition of CRP recognition in the Roche assay and/or CRP cross linking in size exclusion chromatography (P1d-0, P1d-6, P2B-B, P2B-H, P2B-D). Activity was observed with a (3R)-quinuclidinyl configuration. Alkyl linkers with odd numbers of carbons precipitated CRP and no crystals were obtained. All of the complexes crystallised in a body centred orthorhombic space group (I2₁2₁2₁) with one pentamer and five half-ligands in the unique volume of the crystal lattice. Data quality was rather good, often being better than 2 Å resolution. The pentamer separation was generally rather wide and there was little if any evidence of pentamer rotation. As anticipated from modelling, the quinuclidine rings partially occupy the pocket adjacent to Phe66/Gu81, the carboxylate interacts with the protein bound calcium ions and the amino group projects away from the protein surface.

D. Bis(quinuclidine) amino benzyl-phosphonate

In view of the expected higher binding affinity of phosphate or phosphonate for calcium a programme was initiated to produce aminophosphonate compounds. This led to production of a singly protected product (by LCMS), P3A-C with outstanding biological properties. It also produced very good crystals with CRP with data extending to 1.8 Å. The general organisation of the cross linked decamer looks very much like that seen with P2B-B above. However, the geometry of the calcium coordination appears to be tidier, in particular as the amide nitrogens of Asn61 and Gln150 are now in H-bond distance of the phosphonate oxygens. The electron density clearly shows the shape of the quinuclidine ring. There is no clear density for the benzyl protecting group ring or connecting carbon. They must be either highly mobile or absent.

### 8. Results—In Vivo Clearance

### A. Clearance of Ligand

The two best bis(phosphocholine) ligands, BPC6 and BPC8, were both rapidly cleared from the mouse circulation with a half-life (t_{1/2}) of 16.1 and 15.4 minutes respectively. Pk-023 also had a similar plasma t_{1/2} of ˜15 min,

### B. Clearance of CRP/Ligand Complexes Formed In Vitro

Complexes were prepared in vitro using ¹²⁵I-labelled CRP and an excess of ligand, and the mixture was then injected intravenously into mice. Clearance of CRP was monitored by counting of the ¹²⁵I-CRP tracer and stable CRP-ligand complexes were detected by lack of CRP detection in the Roche assay of the plasma samples. The formation of CRP/ligand complexes was confirmed by size exclusion chromatography before use in each in vivo experiment.

CRP-ligand complexes were cleared at the same rate, t_{1/2}˜130 minutes, as CRP alone but the persistence of the preformed complexes in the circulation varied with the different ligands. With Pk-023, the Roche CRP value was suppressed at 15 min but was had returned to control value at 60 min. With P2B_B and with P2B-D peak 1, the Roche value was suppressed for at least 60 min.

The limited stability of the CRP-ligand complexes in vivo was unexpected, since these complexes were all stable in vitro in the absence of free ligand. However, interestingly, the complex with P3A-C, for which there was evidence of more avid binding in vitro, produced complexes that were stable in vivo for at least 6 hours.

### C. Clearance of CRP/Ligand Complexes Formed In Vivo

Subcutaneous injection of human CRP provided circulating CRP concentrations with which to test for effects of our ligands in vivo. (Table 4).

TABLE 4
Circulating CRP concentrations after
subcutaneous of 1 mg CRP in mice

| | 16 h | 18 h | 24 h | |
|---|---|---|---|---|
| Mouse 1 | 58.2 | 44.0 | 24.0 | mg/l |
| Mouse 2 | 69.2 | 57.2 | 32.2 | mg/l |

Eighteen hours after a subcutaneous injection of CRP (1 mg) an i.v. bolus of BPC8 (1 mg) into CRP-treated mice immediately blocked detection of circulating CRP in the Roche assay. At one hour, the Roche assay again detected some CRP and by 3 hours the Roche value was the same as in control mice that had received CRP alone. Although Pk-023 was a much more effective crosslinking ligand in vitro, its in vivo effect was the same as BPC8. The bis(quinuclidine) carboxylate ligand, P2B-B, also generated a CRP:ligand complex in vivo that was stable for at least 1 hour. P2B-H (the P2B-B analogue with a 1,4 cyclohexyl group replacing the benzene linker) produced a complex that was partially stable for at least 3 hours.

### Additional Examples

The invention will now be further illustrated, but not limited, by reference to the specific embodiments described in the following additional examples. Compounds are named using conventional IUPAC nomenclature, or as named by the chemical supplier.

The following synthetic procedures are provided for illustration of the methods used; for a given preparation or step the precursor used may not necessarily derive from the individual batch synthesized according to the step in the description given.

### Analytical Methods

Where examples and preparations cite analytical data, one of the following analytical methods were used unless otherwise specified.

NMR: 400 MHz Bruker Avance III and Bruker Avance Neo.

### LC-MS or HPLC Method:

### Method 1:

MS instrument type: SHIMADZU LC-MS-2020, Column: Kinetex EVO C18 30×2.1 mm, 5 μm, mobile phase A: 0.0375% TFA in water (v/v), B: 0.01875% TFA in Acetonitrile (v/v), gradient: 0.0 min 0% B→0.8 min 60% B→1.20 min 60% B→1.21 min 0% B→1.55 min 0% B flow rate: 1.5 mL/min, oven temperature: 50° C.; PDA detection: 220 nm & 254 nm.

### Method 2:

MS instrument type: Agilent 1200 LC/G1956A MSD, Column: Kinetex EVO C18 2.1×30 mm, 5 μm, mobile phase A: 0.0375% TFA in water (v/v), B: 0.01875% TFA in Acetonitrile (v/v), gradient: 0.0 min 90% B→0.35 min 90% B flow rate: 1.5 mL/min, oven temperature: 50° C.; DAD: 100-1000.

### Method 3:

HPLC instrument type: SHIMADZU LC-20AB, Column: Kinetex C18 LC Column 4.6×50 mm, 5 μm, mobile phase A: 0.0375% TFA in water (v/v), B: 0.01875% TFA in Acetonitrile (v/v), gradient: 0.0 min 0% B→4.20 min 60% B→5.30 min 60% B→5.31 min 0% B→6.00 min 0% B, flow rate: 1.5 mL/min, oven temperature: 50° C.; PDA detection: PDA (220 nm&215 nm&254 nm).

### Method 4:

MS instrument type: SHIMADZU LC-MS-2020, Column: Kinetex EVO C18 30×2.1 mm, 5 μm, mobile phase A: 0.0375% TFA in water (v/v), B: 0.01875% TFA in Acetonitrile (v/v), gradient: 0.0 min 0% B→3.0 min 60% B→3.50 min 60% B→3.51 min 0% B→4.00 min 0% B flow rate: 0.8 mL/min, oven temperature: 50° C.; PDA detection: 220 nm & 254 n.

### Method 5:

MS instrument type: SHIMADZU LC-MS-2020, Column: Kinetex EVO C18 2.1×30 mm, 5 μm, mobile phase A: 0.025% NH3.H2 in Water (v/v), B: Acetonitrile, gradient: 0.0 min 0% B→0.8 min 60% B→1.20 min 60% B→1.21 min 0% B→1.55 min 0% B flow rate: 1.5 mL/min, oven temperature: 40° C.; PDA detection: 220 nm & 254 n.

### Method 6:

HPLC instrument type: SHIMADZU LC-20AB, Column: Kinetex C18 LC Column 4.6×50 mm, 5 μm, mobile phase A: 0.0375% TFA in water (v/v), B: 0.01875% TFA in Acetonitrile (v/v), gradient: 0.0 min 0% B→4.20 min 30% B→5.30 min 30% B→5.31 min 0% B→6.00 min 0% B, flow rate: 1.5 mL/min, oven temperature: 50° C.; PDA detection: PDA (220 nm&215 nm&254 nm).

### Method 7:

MS instrument type: SHIMADZU LC-20AB, Column: Kinetex C18 LC Column 4.6×50 mm, 5 μm, mobile phase A: 0.0375% TFA in water (v/v), B: 0.01875% TFA in Acetonitrile (v/v), gradient: 0.0 min 0% B→2.40 min 30% B→3.70 min 30% B→3.71 min 0% B→4.00 min 0% B flow rate: 1 mL/min, oven temperature: 50° C.; PDA detection: 220 nm & 254 nm.

### Method 8:

MS instrument type: Agilent 1100 LC & Agilent G1956A, Column: Waters XSelect HSS T3 3.5 μm 4.6×50 mm, mobile phase A: 0.0375% TFA in water (v/v), B: 0.01875% TFA in Acetonitrile (v/v), gradient: 0.0 min 0% B→5.00 min 30% B→6.00 min 100% B→6.50 min 100% B→6.51 min 0% B→7.00 min 0% B flow rate: 1 mL/min, oven temperature: 40° C.; PDA detection: 220 nm & 254 n.

### Method 9

MS instrument type: SHIMADZU LCMS-2020, Column: Kinetex EVO C18 2.1×30 mm, 5 μm, mobile phase A: 0.025% NH₃.H₂O in Water (v/v), B: Acetonitrile, gradient: 0.0 mins 5% B→0.8 mins 95% B→1.2 mins 95% B→1.21 mins 5% B→1.55 mins 5% B, flow rate: 1.5 mL/mins, oven temperature: 40° C.; UV detection: 220 nm & 254 nm.

### Method 10

MS instrument type: Agilent 1100 LC & Agilent G1956A, Column: K Waters XSelect HSS T3 3.5 μm 4.6×50 mm, mobile phase A: 0.0375% TFA in water (v/v), B: 0.01875% TFA in Acetonitrile (v/v), gradient: 0.0 mins 0% B→5 mins 30% B→6 mins 100% B→6.5 mins 100% B→6.51 mins 0% B, flow rate: 0.6 mL/mins, oven temperature: 40° C.; UV detection: 220 nm & 254 nm.

### HPLC Method 1:

MS instrument type: SHIMADZU LC-20AB, Column: XBridge® C18 3.5 μm 4.6×150 mm, mobile phase A: 0.0375% TFA in water (v/v), B: 0.01875% TFA in Acetonitrile (v/v), gradient: 0.0 mins 0% B→10.0 mins 60% B→15.0 mins 60% B→15.01 mins 0% B→15.02 mins 0% B→20.0 mins 0% B, flow rate: 1.0 mL/mins, oven temperature: 40° C.; UV detection: 220 nm &215 nm & 254 nm.

### Abbreviations

Where the following abbreviations have been used, the following meanings apply:

ACN or MeCN is acetonitrile,
CDCl₃ is deuterochloroform,
CSA is Camphor-O-sulfonic acid,
D₂O is deuterium oxide,
DCM is dichloromethane,

### DIPEA or DIEA is N,N-diisoproylethylamine,

DMAP is 4-(dimethylamino)pyridine,
DMSO is dimethyl sulfoxide,
EA is ethyl acetate,
EtOH is ethanol,
FA is formic acid,
H₂O is water,
HCl is hydrochloric acid,
HPLC is high performance liquid chromatography,
IPA is isopropyl alcohol,
KHMDS is potassium bis (trimethylsilyl)amide,
KOH is potassium hydroxide,
LCMS is liquid chromatography mass spectrometry,
MeOH is methanol,
MTBE is methyl tert butyl ether,
N₂ is nitrogen,
Na₂SO₄ is sodium sulfate;
NH₃ is ammonia,
NH₄HCO₃ is ammonium bicarbonate,
NMR is nuclear magnetic resonance,
PDA is photodiode array detector,
SFC is supercritical fluid chromatography,
TBTU is 2-(1H-Benzotriazole-1-yl)-1,1,3,3-tetramethylaminium tetrafluoroborate,
TEA is triethylamine,
TFA is trifluoroacetic acid,
THF is tetrahydrofuran and
TLC is thin layer chromatography.

### Preparations

### Preparation of Compound 2: [(3S)-quinuclidin-3-yl] 4-bromobenzenesulfonate

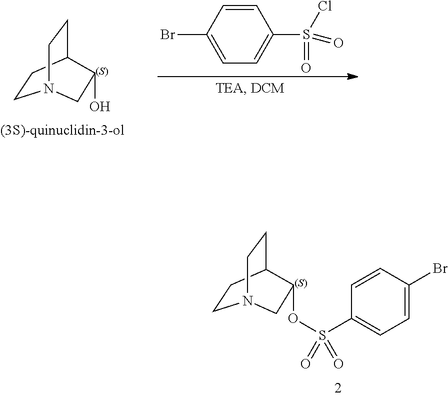

To a solution of (3S)-quinuclidin-3-ol (2.00 g, 15.73 mmol, 1.00 eq), DMAP (19.21 mg, 157.30 gmol, 0.01 eq) and TEA (4.78 g, 47.19 mmol, 6.54 mL, 3.00 eq) in DCM (40.00 mL) was added 4-bromobenzenesulfonyl chloride (6.03 g, 23.60 mmol, 1.50 eq) at 0° C. The mixture was stirred for 16 hours at 20° C. The mixture was washed with sat. NaHCO₃ (100 mL). The sat. NaHCO₃ layer was extracted with EA (100 mL×2). The combined organic layers were dried over Na₂SO₄ and concentrated under reduced pressure to give a crude product as a yellow oil. The yellow oil was purified by chromatography on silica gel eluted with DCM:MeOH=30:1 to give [(3S)-quinuclidin-3-yl] 4-bromobenzenesulfonate (3.20 g, 8.32 mmol, 52.88% yield, 90% purity) as a yellow solid, which was analyzed by ¹HNMR.

¹H NMR: (400 MHz, CDCl₃) δ=7.81-7.57 (m, 4H), 4.65-4.49 (m, 1H), 3.04 (dd, J=8.4, 15.2 Hz, 1H), 2.89-2.48 (m, 5H), 1.93 (d, J=2.8 Hz, 1H), 1.80-1.71 (m, 1H), 1.61 (tdd, J=4.6, 9.5, 13.9 Hz, 1H), 1.47-1.22 (m, 2H).

### Preparation of Diastereomers 4A and 4B

### Isomer 4A: (R)-methyl 2-((diphenylmethylene)amino)-2-((3R)-quinuclidin-3-yl)acetate

### Isomer 4B:_(S)-methyl 2-((diphenylmethylene)amino)-2-((3R)-quinuclidin-3-yl)acetate

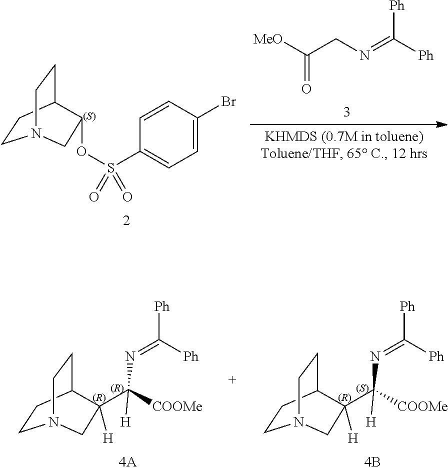

To a solution of (3S)-quinuclidin-3-yl 4-bromobenzenesulfonate (63 g, 182 mmol) and methyl 2-[(diphenylmethylidene)amino]acetate (92.2 g, 364 mmol) in Toluene (578 mL) and THF (186 mL) was added KHMDS (0.70 M in toluene, 520 mL) under N₂ and the reaction was stirred at 65° C. for 12 hours. The reaction mixture was cooled, poured into water (1.00 L) and ethyl acetate (1500 mL) was added. The phases were separated and the aqueous phase was extracted with ethyl acetate (3×1.00 L). The organic layer was washed with brine (2×500 mL), dried over Na₂SO₄, filtered and concentrated in vacuo. The crude mixture (113 g) was obtained as a dark brown oil and used directly in the next step. Selected NMR data of crude material showed dr (R,R) (R,S)=2.3:1 ¹H-NMR: 400 MHz, DMSO-d₆: crude selected δ ppm 4.08 (d, J=8.8 Hz, 2H), 3.95 (d, J=10.2 Hz, 1H).

### Preparation of 5. 2 (+)-CSA Salt (R,R)

### (R)-methyl 2-amino-2-((3R)-quinuclidin-3-yl)acetate bis(((1S,4R)-7,7-dimethyl-2-oxobicyclo[2.2.1]heptan-1-yl)methanesulfonate)

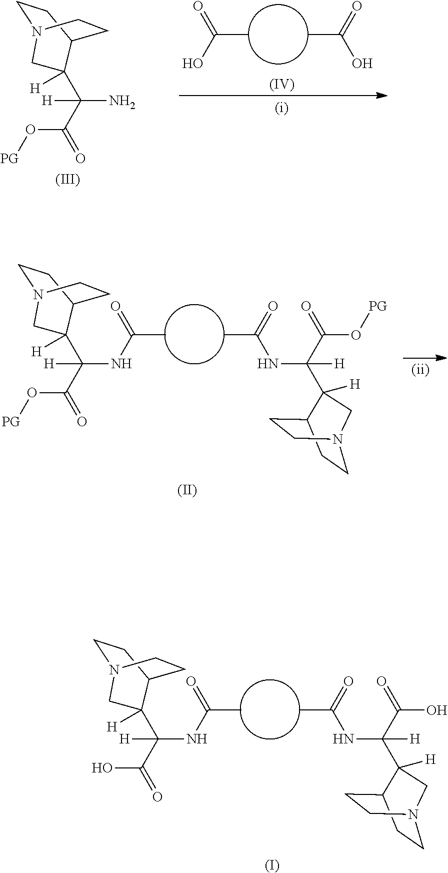

To a solution of the crude reaction mixture of 4A and 4B (105 g, 177 mmol) in IPA (700 mL) was added H₂O (3.21 g, 178 mmol) and the reaction was warmed to 45° C. A solution of (+) CSA (103 g, 442 mmol) in IPA (300 mL) was added and the reaction continued stirring at 45° C. for 12 hrs. The reaction mixture was cooled to 25° C. and filtered to obtain a white solid. The solid was washed with IPA (100 mL) and MTBE (100 mL) and dried under vacuum to obtain the title compound as a white solid (62.0 g, 93.5 mmol, 52.9% yield). ¹H-NMR: 400 MHz, DMSO-d₆: δ ppm 9.62-9.58 (br, s, 1H), 8.51 (br, s, 3H), 4.25-4.22 (d, J=10.4 Hz, 1H), 3.78 (s, 3H), 3.25-3.23 (m, 5H), 2.90-2.86 (d, J=14.8 Hz, 2H), 2.66 (m, 2H), 2.41-2.37 (d, J=14.8 Hz, 3H), 1.95-1.93 (m, 2H), 1.85-1.78 (m, 11H), 1.30-1.27 (m, 4H), 1.04 (s, 6H), 0.74 (s, 6H).

### Confirmation of Stereochemistry for Compound 5. 2 (+)-CSA Salt (R,R)

20 mg compound 4A was dissolved in 1.3 mL dichloromethane/cyclohexane/methanol (5:5:3). The solution was kept in a half sealed 4 mL vial and evaporated slowly at room temperature. Crystals were observed in the second day and a crystal was selected for X-ray crystallographic analysis.

The crystal was a colorless needle with the following dimensions: 0.10×0.02×0.02 mm 3. The symmetry of the crystal structure was assigned the monoclinic space group P2₁ with the following parameters: a=7.0236(2) Å, b=26.8204(6) Å, c=18.0068(5) Å, α=90°, β=99.114(3)°, γ=90°, V=3349.22(16) Å 3, Z=4, Dc=1.315 g/cm³, F(000)=1424.0, μ(Cu Kα)=1.918 mm⁻¹ and T=293(2) K using a Rigaku Oxford Diffraction XtaLAB Synergy four-circle diffractometer equipped with a HyPix-6000HE area detector. Cryogenic system: Oxford Cryostream 800 Cu: λ=1.54184 Å, 50W, Micro focus source with multilayer mirror (μ-CMF). Distance from the crystal to the CCD detector: d=35 mm Tube Voltage: 50 kV Tube Current: 1 mA.

The absolute configuration of 5. 2 (+)-CSA salt was assigned (R,R).

Alternatively, compound 4A (R,R) may be isolated according to the following method:

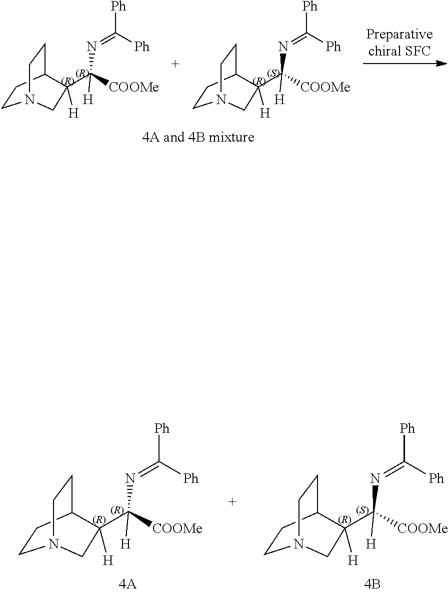

The crude reaction mixture of 4A and 4B was purified by silica gel column chromatography eluting with EA:MeOH 40:1 to 10:1) to afford a mixture of diastereomers. The diastereomers were resolved by chiral prep-SFC (column: DAICEL CHIRALPAK IG (250 mm×50 mm, 10 μm); mobile phase: [0.1% NH₃.H₂O EtOH]; B %: 45%; 320 min) to afford the two diastereomers of compound 4A (6.00 g, 16.5 mmol) as a brown oil and compound 4B (RS) (9.00 g, 24.8 mmol) as a brown oil.

### Diastereomer 1 (RR): (R)-methyl 2-((diphenylmethylene)amino)-2-((3R)-quinuclidin-3-yl)acetate

¹H-NMR 400 MHz (DMSO-d₆): δ ppm: 7.64-7.34 (m, 8H), 7.25-7.09 (m, 2H), 4.09 (d, J=8.8 Hz, 1H), 3.60 (s, 3H), 2.92-2.77 (m, 1H), 2.73-2.60 (m, 2H), 2.55 (br d, J=6.4 Hz, 1H), 2.43-2.21 (m, 3H), 1.66-1.33 (m, 3H), 1.19 (br d, J=5.2 Hz, 2H).

SFC: Rt=1.633 min, 100%

### Diastereomer 2 (RS): (S)-methyl 2-((diphenylmethylene)amino)-2-((3R)-quinuclidin-3-yl)acetate

¹H-NMR 400 MHz (DMSO-d₆): δ ppm: 7.62-7.35 (m, 8H), 7.18 (dd, J=1.6, 7.4 Hz, 2H), 3.96 (d, J=10.0 Hz, 1H), 3.65 (s, 3H), 2.95-2.80 (m, 1H), 2.65 (br t, J=7.6 Hz, 2H), 2.48 (br s, 1H), 2.4-2.23 (m, 2H), 2.17 (br dd, J=7.2, 13.6 Hz, 1H), 1.65-1.40 (m, 3H), 1.13-1.05 (m, 1H), 0.96-0.78 (m, 1H).

SFC: Rt=1.854 min, 100%

Alternatively, compounds 4A and 4B may be separated using preparative TLC as described below: A mixture of 4A and 4B were purified by Preparative TLC (EtOAc:MeOH (NH₃, 7M)=10:1) to give 202.25 mg of 4A (91.7% purity, 98.6% ee.) as a yellow solid, 114.50 mg of 4B (97.7% purity, 94.3% ee.) as a yellow oil.

LCMS MS m/z 363.2 [M+H]⁺

4A: ¹H NMR: (400 MHz, CDCl₃): δ ppm 7.66-7.58 (m, 2H), 7.52-7.45 (m, 3H), 7.43-7.31 (m, 3H), 7.19 (dd, J=1.6, 7.4 Hz, 2H), 4.22 (d, J=8.3 Hz, 1H), 3.73-3.68 (m, 3H), 3.17-3.03 (m, 1H), 2.92-2.82 (m, 2H), 2.64-2.51 (m, 3H), 1.77-1.55 (m, 3H), 1.52-1.40 (m, 1H), 1.37-1.25 (m, 1H).

4B: ¹H NMR (400 MHz, CDCl₃): δ ppm 7.65-7.56 (m, 2H), 7.54-7.44 (m, 3H), 7.43-7.29 (m, 3H), 7.20 (dd, J=2.9, 6.4 Hz, 2H), 4.08 (d, J=10.0 Hz, 1H), 3.76-3.72 (m, 3H), 3.24-3.09 (m, 1H), 3.00-2.80 (m, 2H), 2.61-2.38 (m, 3H), 1.81-1.58 (m, 3H), 1.28-1.15 (m, 1H), 1.12-1.00 (m, 1H)

Alternatively, the HCl salt of compound 5 may be obtained by following the procedure below: To a solution of the above-prepared 4A stereoisomer (390.00 mg, 1.08 mmol) in THF (6 mL) was added HCl (12 M (aq), 780.09 μL, 37% purity) at 0° C. The reaction mixture was stirred for 1 hour at 0° C. The mixture was concentrated to remove THF. To the residue was added methyl tertiary butyl ether (20 mL) and water (20 mL). The aqueous layer was concentrated under reduced pressure to give (R)-methyl 2-amino-2-((3R)-quinuclidin-3-yl)acetate (250.00 mg, crude, 2HCl salt) as a yellow solid.

Preparation of Compound 5 Free Parent from Compound 5 CSA Salt

### (R)-methyl 2-amino-2-((3R)-quinuclidin-3-yl)acetate

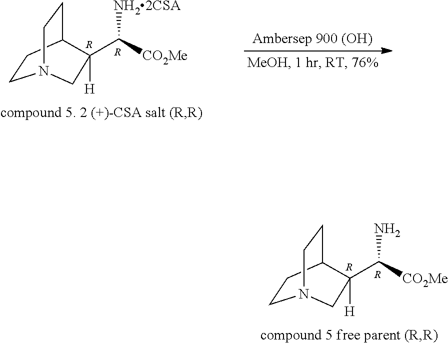

To a suspension of Ambersep 900 (470 g) in MeOH (900 mL) was added methyl (2R)-2-amino-2-[(3R)-1-azabicyclo[2.2.2]octan-3-yl]acetate bis (+) Camphorsulfonic acid salt (Preparation 1, 47.0 g, 70.9 mmol) and the mixture was stirred at 20° C. under N₂ for 1 hour. The reaction mixture was filtered and concentrated in vacuo to afford the title compound (11.0 g, 55.5 mmol, 78.3% yield) as a yellow oil. ¹H-NMR 400 MHz (DMSO-d₆): δ ppm: 7.64-7.34 (m, 8H), 7.25-7.09 (m, 2H), 4.09 (d, J=8.8 Hz, 1H), 3.60 (s, 3H), 2.92-2.77 (m, 1H), 2.73-2.60 (m, 2H), 2.55 (br d, J=6.4 Hz, 1H), 2.43-2.21 (m, 3H), 1.66-1.33 (m, 3H), 1.19 (br d, J=5.2 Hz, 2H).

The hydrochloride salt of compound 5 may also be converted to the free parent using the Ambersep 900 method described above.

### Synthesis of Additional Examples

### Additional Example 1, Compound ID APL-2191 or P2B-B

### (R,2R,2′R)-2,2′-(terephthaloylbis(azanediyl))bis(2-((R)-quinuclidin-3-yl)acetic acid)

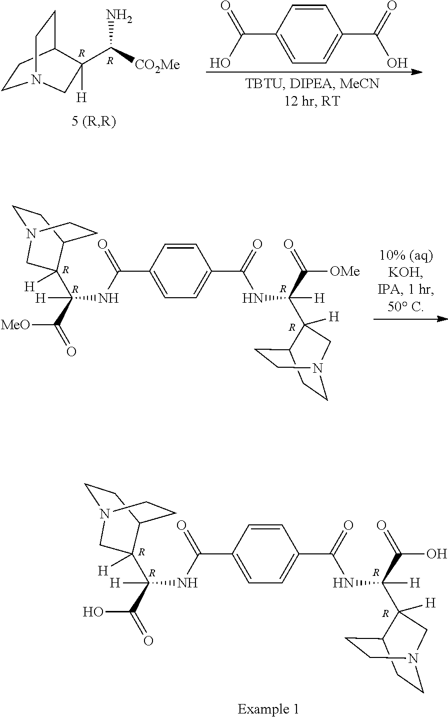

Step 1

To a methyl (2R)-2-amino-2-[(3R)-1-azabicyclo[2.2.2]octan-3-yl]acetate

(Preparation 2, 330 g, 333 mmol, 20% solution in MeCN) and benzene-1,4-dicarboxylic acid (20.50 g, 123 mmol) in MeCN (1.30 L) was added TBTU (88.2 g, 275 mmol) under N₂, followed by DIEA (65.3 g, 505 mmol, 88.0 mL). The reaction was stirred at 25° C. for 12 hrs. The reaction mixture was concentrated in vacuo to afford a crude yellow oil that was taken directly on to the next step.

Step 2

To a solution of the crude reaction mixture from Step 1 (64.9 g, 123 mmol) in IPA (1.07 L) was added KOH (69.2 g, 123 mmol, 1.07 L, 10% aqueous) and the reaction was stirred at 50° C. under N₂ for 1 hour. The reaction mixture was filtered and the mother liquor was extracted with ethyl acetate (2×300 mL). The aqueous layer was adjusted to pH=4-5 with formic acid and stirred for 12 hours. The resultant white solid was filtered and stirred in water (740 mL) at 90° C. for 2 hours before cooling to 25° C. The solid was filtered, washed with water (2×300 mL) and dried under vacuum to afford the title compound as a white solid (31.4 g, 48.6 mmol, 39.5% yield).

¹H-NMR 400 MHz (D₂O): δ ppm: 7.84 (s, 4H), 4.53 (br d, J=10.8 Hz, 2H), 3.54-3.36 (m, 2H), 3.35-3.26 (m, 8H), 3.07 (br dd, J=7.6, 12.2 Hz, 2H), 2.53-2.51 (m, 2H), 2.19-1.98 (m, 4H), 1.94-1.91 (m, 6H).

LCMS (Method 1): Rt=2.275 min, MS m/z [M+H]⁺ 499.4, theoretical mass: 498.6

HPLC (Method 1): Rt=3.908 min, 99.7%

Elemental Analysis: C, 45.89%; H, 7.96%; N, 8.18%, theoretical+10H₂O: C, 46.01%; H, 8.02%; N, 8.25%. 15 mg compound 4A was dissolved in 1.2 ml ethanol/H₂O (1:1) at 60° C. The solution was filtered through 0.45 μm microporous filter and kept in a sealed 4 ml vial at room temperature. Needle crystals were observed in the solution and a crystal selected for X-ray crystallographic analysis

The crystal was a colorless needle with the following dimensions: 0.30×0.04×0.04 mm3. The symmetry of the crystal structure was assigned the orthorhombic space group C222₁ with the following parameters: a=15.9948(2) Å, b=22.5673(3) Å, c=9.5013(2) Å, α=90°, β=90°, γ=90°, V=3429.58(10) Å 3, Z=4, Dc=1.280 g/cm 3, F(000)=1424.0, μ(Cu Kα)=0.889 mm⁻¹, and T=110(14) K using Rigaku Oxford Diffraction XtaLAB Synergy four-circle diffractometer equipped with a HyPix-6000HE area detector. Cryogenic system: Oxford Cryostream 800 Cu: λ=1.54184 Å, 50W, Micro focus source with multilayer mirror (μ-CMF). Distance from the crystal to the CCD detector: d=35 mm Tube Voltage: 50 kV Tube Current: 1 mA.

The absolute configuration of Example 1 was assigned (R,R, R,R)

### Preparation of Additional Example 1 HCl Salt

To the suspension of APL-2191 (30.9 g, 45.5 mmol, 1 eq, 10H₂O) in H₂O (760 mL) and EtOH (760 mL) was added HCl (12 M, 7.61 mL, 2.01 eq) at 25° C. and stirred for 12 hr. The reaction mixture was concentrated under vacuum. APL-2191.2HCl (28.2 g, 39.0 mmol, 85.7% yield, 10H₂O) was obtained as a crystalline off-white solid.

LCMS (Method 1): Rt=2.300 min, MS m/z 250.1 [M+H/2]⁺

HPLC (Method 2): Rt=3.889 min, 99.3%

¹H-NMR 400 MHz (D₂O): δ ppm: 7.85 (s, 4H), 4.67 (d, J=11.2 Hz, 2H), 3.63-3.50 (m, 2H), 3.41-3.22 (m, 8H), 3.10 (ddd, J=1.8, 6.8, 13.2 Hz, 2H), 2.73-2.58 (m, 2H), 2.30-2.16 (m, 4H), 2.10-1.88 (m, 6H).

The following Additional Examples were prepared using the same procedure as described for Example 1 (see General method below) using the appropriate dicarboxylic acid as described for each Example, and compound 5 (R,R). The Examples were purified as individually described for Step 1 and Step 2.

### General Method for Additional Examples 2-11

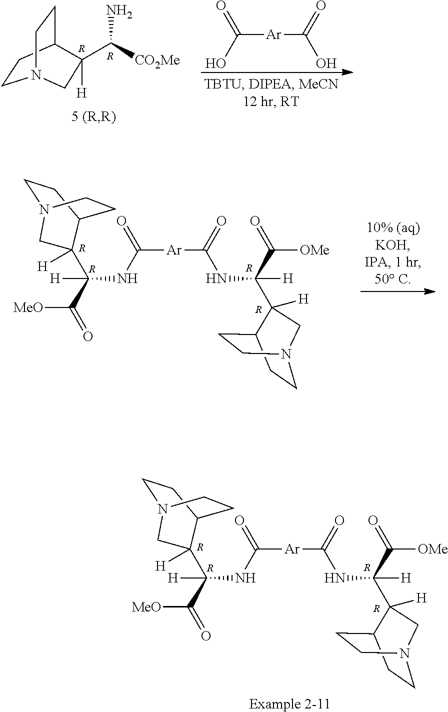

Step 1

To a solution of compound 5 (2.70 eq) in ACN (10 V) was added TBTU (2.23 eq) and the appropriate carboxylic acid (1 eq) at 20° C. under nitrogen. DIEA (4.11 eq) was added to the mixture, and the mixture was stirred at 20° C. for 6 hrs under N₂. The reaction mixture was concentrated under vacuum and purified as described for each Example.

Step 2

To a solution of the bis-methyl esters (1.00 eq) in IPA (20.0 V) was added aqueous KOH (10.0%, 10.0 eq) at 20° C. The mixture was stirred at 50° C. for 1 hr, cooled to room temperature and purified as described for each Example.

### Additional Example 2, Compound ID APL-6968

### (R,2R,2′R)-2,2′-((pyridine-2,5-dicarbonyl)bis(azanediyl))bis(2-((R)-quinuclidin-3-yl)acetic acid)

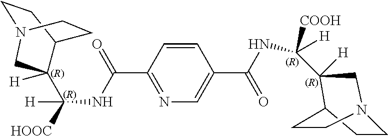

Additional Example 2 was prepared according to the General Method using pyridine-2,5-dicarboxylic acid.

Step 1

The residue was purified by prep-HPLC (column: Phenomenex Gemini-NX C18 75×30 mm, 3 μm; mobile phase: [water (0.225% FA)-ACN]; B %: 1%-20%, 7 min) to give the bis methyl ester (330 mg, 524 umol, 43.8% yield, 91.2% purity, FA) as a white solid.

¹H-NMR 400 MHz (CDCl₃): δ ppm: 8.93 (br s, 1H), 8.82-8.60 (m, 2H), 8.50-8.40 (m, 2H), 8.26-8.24 (m, 1H), 8.02-8.01 (m, 1H), 4.92-4.84 (m, 1H), 4.82-4.73 (m, 1H), 3.80 (d, J=14.0 Hz, 6H), 3.28-3.12 (m, 7H), 2.31-2.12 (m, 12H), 1.99-1.77 (m, 10H).

LCMS (Method 1) Rt=0.685 min, MS m/z [M+H]⁺ 528.2

Step 2

The residue was purified by prep-HPLC (column: Waters Atlantis T3 150×30 mmx, 5 μm; mobile phase: [water (0.225% FA)-ACN]; B %: 1%-20%, 10 min) to afford Additional Example 2 (74.0 mg, 132 gmol, 97.4% purity, FA) as a white solid.

MS (Method 8): MS m/z 499.9 [M+H]⁺, theoretical mass: 499.2

HPLC (Method 1): Rt=2.31 min

¹H-NMR 400 MHz (D₂O): δ ppm: 8.97 (d, J=1.6 Hz, 1H), 8.43 (s, 1H), 8.33-8.31 (m, 1H), 8.13 (d, J=8.0 Hz, 1H), 4.56 (dd, J=8.4, 10.4 Hz, 2H), 3.60-3.52 (m, 2H), 3.39-3.24 (m, 8H), 3.13-3.04 (m, 2H), 2.62-2.51 (m, 2H), 2.28-2.19 (m, 4H), 2.05-1.89 (m, 6H).

### Additional Example 3, Compound ID APL-6969

### (R,2R,2′R)-2,2′-((pyrazine-2,5-dicarbonyl)bis(azanediyl))bis(2-((R)-quinuclidin-3-yl)acetic acid)

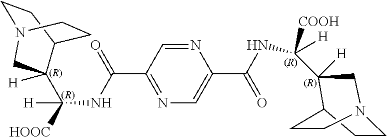

Additional Example 3 was prepared according to the General Method using pyrazine-2,5-dicarboxylic acid.

Step 1

The residue was purified by prep-HPLC (column: Waters Xbridge 150×25 mm, 5 μm; mobile phase: [water (10 mM NH₄HCO₃)-ACN]; B %: 14%-44%, 9 min) to give the bis methyl ester (90.0 mg, 124 gmol, 10.4% yield, 72.7% purity) as a white solid.

LCMS (Method 1): Rt=0.704 min, MS m/z 529.3 [M+H]⁺

Step 2

The mixture was filtered, FA (aq, 20% in water) was added to adjust the mixture to pH=7˜8 and the mixture was purified by prep-HPLC (column: Waters Xbridge 150×25 mm, 5 μm; mobile phase: [water (10 mM NH₄HCO₃)-ACN]; B %: 1%-10%, 9 min) to give Additional Example 3 (51.0 mg, 98.0 gmol, 57.5% yield, 96.0% purity) as a white solid.

MS (Method 2): MS [M+H]⁺ 501.1, theoretical mass: 500.2

HPLC (Method 3): Rt=0.824 min

¹H-NMR 400 MHz (D₂O): δ ppm: 9.21 (s, 2H), 8.38 (br s, 4H), 4.55 (d, J=3.60 Hz, 2H), 3.53-3.48 (m, 2H), 3.40-3.20 (m, 8H), 3.10-3.01 (m, 2H), 2.63-2.52 (m, 2H), 2.21 (br s, 4H), 2.07-1.85 (m, 6H).

### Additional Example 4, Compound ID APL-6970

### (R,2R,2′R)-2,2′-((pyridazine-3,6-dicarbonyl)bis(azanediyl))bis(2-((R)-quinuclidin-3-yl)acetic acid)

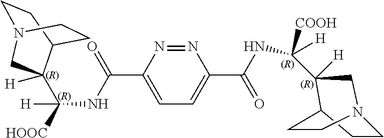

Additional Example 4 was prepared according to the General Method using pyridazine-3,6-dicarboxylic acid.

Step 1

The residue was purified by prep-HPLC (column: Phenomenex Gemini-NX C18 75×30 mm, 3 μm; mobile phase: [water (10 mM NH₄HCO₃)-ACN]; B %: 5%-35%, 8 min) to give the bis methyl ester (110 mg, 144 gmol, 22.0% yield, 81.1% purity) as a white solid.

¹H-NMR 400 MHz (CDCl₃): δ ppm: 7.86 (d, J=8.4 Hz, 2H), 7.72 (s, 1H), 7.65 (dd, J=1.6, 8.0 Hz, 1H), 7.40 (d, J=8.4 Hz, 2H), 7.29 (d, J=8.0 Hz, 1H), 6.55-6.50 (m, 2H), 4.96-4.91 (m, 2H), 3.79 (s, 6H), 3.16-3.05 (m, 3H), 3.02-2.69 (m, 11H), 2.31 (s, 3H), 2.09-1.87 (m, 9H), 1.79-1.63 (m, 5H).

LCMS (Method 1): Rt=0.807 min, MS m/z 617.3

Step 2

The residue was purified by prep-HPLC (column: Waters Atlantis T3 150×30 mm, 5 μm; mobile phase: [water (0.225% FA)-ACN]; B %: 1%-20%, 10 min) to give Additional Example 4 (87.0 mg, 154 gmol, 32.7% yield, 97.3% purity, FA) as a white solid.

LCMS (Method 8): Rt=2.296 min, MS m/z 501.4 [M+H]⁺, theoretical mass: 500.3

¹H-NMR 400 MHz (D₂O): δ ppm: 8.41 (s, 2H), 8.35 (s, 0.25H), 4.62 (d, J=10.8 Hz, 2H), 3.62-3.52 (m, 2H), 3.42-3.22 (m, 8H), 3.19-3.09 (m, 2H), 2.68-2.55 (m, 2H), 2.30-2.18 (m, 4H), 2.09-1.86 (m, 6H).

### Additional Example 5, Compound ID APL-6971

### (R)-2-(4′-(((R)-carboxy((R)-quinuclidin-3-yl)methyl)carbamoyl)-[1,1′-biphenyl]-4-ylcarboxamido)-2-((R)-quinuclidin-3-yl)acetic acid

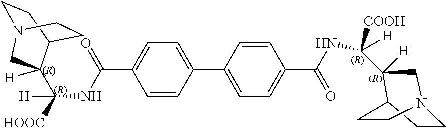

Additional Example 5 was prepared according to the General Method using [1,1′-biphenyl]-4,4′-dicarboxylic acid.

Step 1

The crude material was obtained as a colorless liquid and taken on directly to the next step.

LCMS (Method 1) Rt=0.789 min, MS m/z 603.4 [M+H]⁺

Step 2

The mixture was filtered, and the FA (aq, 20% in water) was added the mixture adjust to pH=7˜8. Purified by prep-HPLC (column: Waters Xbridge 150×25 mm, 5 μm; mobile phase: [water (10 mM NH₄HCO₃)-ACN]; B %: 1%-10%, 9 min) to give Additional Example 5 (37.0 mg, 63.7 gmol, 15.5% yield, 99.0% purity) as a white solid.

LCMS (Method 4): Rt=1.43 min, MS m/z 575.3 [M+H]⁺, theoretical mass: 574.2

¹H-NMR 400 MHz (D₂O): δ ppm: 7.89-7.77 (m, 8H), 4.55 (d, J=10.8 Hz, 2H), 3.59-3.50 (m, 2H), 3.42-3.18 (m, 8H), 3.13-3.02 (m, 2H), 2.58-2.48 (m, 2H), 2.30-2.15 (m, 4H), 2.09-1.84 (m, 6H).

### Additional Example 6, Compound ID APL-6972

### (R)-2-(4′-(((R)-carboxy((R)-quinuclidin-3-yl)methyl)carbamoyl)-2′-methyl-[1,1′-biphenyl]-4-ylcarboxamido)-2-((R)-quinuclidin-3-yl)acetic acid

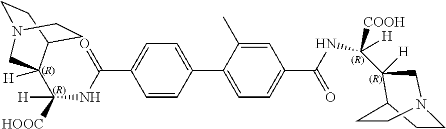

Additional Example 6 was prepared according to the General Method using 2-methyl-[1,1′-biphenyl]-4,4′-dicarboxylic acid.

Step 1

The residue was purified by prep-HPLC (column: Phenomenex Gemini-NX C18 75×30 mm, 3 μm; mobile phase: [water (10 mM NH₄HCO₃)-ACN]; B %: 5%-35%, 8 min) to give the bis ester (110 mg, 144 gmol, 22.0% yield, 81.1% purity) as a white solid.

LC-MS (Method 1): Rt=0.807 min, MS m/z 617.3 [M+H]⁺

¹H-NMR 400 MHz (CDCl₃): δ ppm: 7.86 (d, J=8.4 Hz, 2H), 7.72 (s, 1H), 7.65 (dd, J=1.6, 8.0 Hz, 1H), 7.40 (d, J=8.4 Hz, 2H), 7.29 (d, J=8.0 Hz, 1H), 6.55-6.50 (m, 2H), 4.96-4.91 (m, 2H), 3.79 (s, 6H), 3.16-3.05 (m, 3H), 3.02-2.69 (m, 11H), 2.31 (s, 3H), 2.09-1.87 (m, 9H), 1.79-1.63 (m, 5H).

Step 2

The residue was purified by prep-HPLC (column: Waters Atlantis T3 150×30 mm, 5 μm; mobile phase: [water (0.225% FA)-ACN]; B %: 1%-20%, 10 min) to give Additional Example 6 (FA salt, 16 mg, 5.44 gmol, 4.13% yield, 95.0% purity) as a white solid.

LCMS (Method 4): Rt=1.597 min, MS m/z 589.3 [M+H]⁺, theoretical mass: 588.3

¹H-NMR 400 MHz (D₂O+DMSO): δ ppm: 8.24 (s, 1H), 7.90-7.80 (m, 2H), 7.71 (s, 1H), 7.68-7.62 (m, 1H), 7.43 (br d, J=8.4 Hz, 2H), 7.34-7.27 (m, 1H), 3.35-3.32 (m, 2H), 3.26-3.05 (m, 9H), 2.91-2.83 (m, 2H), 2.22 (s, 3H), 2.15-2.07 (m, 4H), 1.92-1.68 (m, 7H).

### Additional Example 7, Compound ID APL-6973

### (R,2R,2′R)-2,2′-((naphthalene-2,6-dicarbonyl)bis(azanediyl))bis(2-((R)-quinuclidin-3-yl)acetic acid)

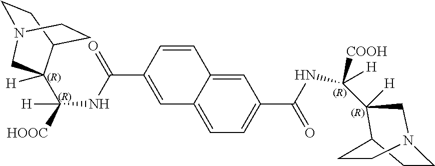

Additional Example 7 was prepared according to General Method 1 using naphthalene-2,6-dicarboxylic acid.

Step 1

The residue was purified by prep-HPLC (column: 3_Phenomenex Luna C18 75×30 mm, 3 μm; mobile phase: [water (0.1% TFA)-ACN]; B %: 5%-35%, 7 min), and the mixture was lyophilized to give the bis ester (160 mg, 277 gmol, 60.0% yield) as a white solid.

LC-MS (Method 1): Rt=0.770 min, MS m/z [M+H]+ 577.4

Step 2

The mixture was filtered, and the FA (aq, 20% in water) was added the mixture adjust to pH 7˜8. The residue was purified by prep-HPLC (column: Waters Xbridge 150×25 mm, 5 μm; mobile phase: [water (10 mM NH4HCO3)-ACN]; B %: 1%-10%, 9 min) to give Additional Example 7 (35.0 mg, 62.0 gmol, 22.0% yield, 96.0% purity) as a white solid.

LCMS (Method 5): Rt=0.282 min, MS m/z 549.1 [M+H]⁺, theoretical mass: 548.2

HPLC (Method 6): Rt=1.624 min

¹H-NMR 400 MHz (D₂O): δ ppm: 8.23 (s, 2H), 7.95 (d, J=10.0 Hz, 2H), 7.77 (d, J=10.0 Hz, 2H), 4.59 (d, J=11.2 Hz, 2H), 3.64-3.52 (m, 2H), 3.44-3.23 (m, 10H), 3.15-3.04 (m, 2H), 2.63-2.52 (m, 2H), 2.31-2.18 (m, 5H), 2.09-1.86 (m, 7H).

### Additional Example 8, Compound ID APL-6974

### (R,2R,2′R)-2,2′-((2,5-dimethylterephthaloyl)bis(azanediyl))bis(2-((R)-quinuclidin-3-yl)acetic acid)

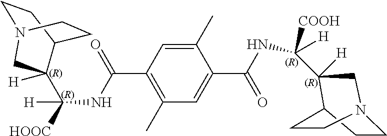

Additional Example 8 was prepared according to the General Method using 2,5-dimethylbenzene-1,4-dicarboxylic acid.

Step 1

The crude product was triturated with ACN (5 mL) and MeOH (3 mL) at 20° C. for 10 min and filtered to give the bis ester (114 mg, 185 gmol, 17.9% yield, 90.1% purity) as a white solid.

LCMS (Method 1): Rt=0.771 min, MS m/z 555.3 [M+H]⁺,

¹H-NMR 400 MHz (DMSO): δ ppm: 8.75 (d, J=6.8 Hz, 1H), 7.18 (br s, 1H), 4.50-4.46 (m, 1H), 3.68 (s, 3H), 3.17-2.91 (s, 12H), 2.78-2.68 (m, 1H), 2.30 (s, 3H), 2.20-2.19 (m, 1H), 1.96-1.90 (m, 1H), 1.83-1.68 (m, 2H), 1.74-1.53 (m, 2H), 1.16 (d, J=6.0 Hz, 1H).

Step 2

The residue was purified by prep-HPLC (column: Waters Atlantis T3 150×30 mm, 5 μm; mobile phase: [water (0.225% FA)-ACN]; B %: 1%-20%, 10 min) to give Additional Example 8 (10.0 mg, 17.1 gmol, 9.50% yield, 98.1% purity, FA) as a white solid.

LCMS (Method 8): Rt=2.773 min, MS m/z 527.3 [M+H]⁺, theoretical mass: 526.3

¹H-NMR 400 MHz (D₂O+DMSO): δ ppm: 7.16 (s, 2H), 4.34 (br d, J=10.4 Hz, 2H), 3.41-3.33 (m, 2H), 3.22-3.10 (m, 7H), 2.94-2.88 (m, 2H), 2.36-2.27 (m, 3H), 2.21 (s, 6H), 2.16-2.01 (m, 4H), 1.90-1.70 (m, 6H).

LCMS m/z 527.3 [M+H]⁺, theoretical mass: 526.3, Rt=2.77 minutes, 100%

### Additional Example 9, Compound ID APL-6975

### (R,2R,2′R)-2,2′-((2-methylterephthaloyl)bis(azanediyl))bis(2-((R)-quinuclidin-3-yl)acetic acid)

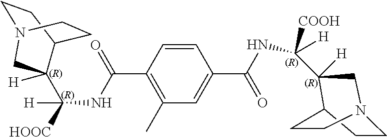

Additional Example 9 was prepared according to the General Method using 2-methylbenzene-1,4-dicarboxylic acid.

Step 1

The residue was purified by prep-HPLC (column: Phenomenex luna C18 150×25 mm, 10 μm; mobile phase: [water (0.225% FA)-ACN]; B %: 0%-20%, 10 min) to give the bis ester (500 mg, 647 gmol, 23.3% yield, 70.0% purity) as a white solid.

LCMS (Method 1): Rt=0.707 min, MS m/z 541.2 [M+H]⁺

Step 2

The mixture was filtered, and FA (aq, 20% in water) was added the mixture adjust to pH 7˜8. The mixture was purified by prep-HPLC (column: Waters Xbridge 150×25 mm, 5 μm; mobile phase: [water (10 mM NH₄HCO₃)-ACN]; B %: 1%-10%, 9 min) to give Additional Example 9 (116 mg, 202.26 gmol, 54.67% yield, 97.4% purity, FA) as a white solid.

LCMS (Method 8): Rt=2.353 min, MS m/z 513.0 [M+H]⁺, theoretical mass: 512.3

¹H-NMR 400 MHz (D₂O): δ ppm: 8.38 (m, 1H), 7.64-7.59 (m, 2H), 7.42 (d, J=8.0 Hz, 1H), 4.53-4.48 (m, 2H), 3.60-3.49 (m, 2H), 3.39-3.21 (m, 8H), 3.12-2.99 (m, 2H), 2.54-2.40 (m, 2H), 2.35 (s, 3H), 2.30-2.14 (m, 4H), 2.04-1.86 (m, 6H).

### Additional Example 10, Compound ID APL-6976

### (R,2R,2′R)-2,2′-((2,5-bis(benzyloxy)terephthaloyl)bis(azanediyl))bis(2-((R)-quinuclidin-3-yl)acetic acid)

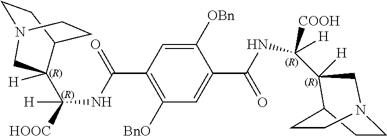

Additional Example 10 was prepared according to the General Method using 2,5-bis(benzyloxy)benzene-1,4-dicarboxylic acid.

Step 1

The residue was purified by prep-HPLC (column: Phenomenex luna C18 150×25 mm, 10 μm; mobile phase: [water (0.225% FA)-ACN]; B %: 11%-41%, 10 min to give the bis ester (180 mg, 211 gmol, 39.9% yield, 92.1% purity, FA) as a white solid.

¹H-NMR 400 MHz (CDCl₃): δ ppm: 8.52 (d, J=8.4 Hz, 2H), 8.37 (s, 1H), 7.93 (s, 2H), 7.58-7.40 (m, 10H), 5.27-5.17 (m, 4H), 4.79-4.75 (m, 2H), 3.69 (s, 6H), 3.34-3.14 (m, 6H), 3.07-2.95 (m, 2H), 2.90-2.75 (m, 4H), 2.11-2.01 (m, 2H), 1.99-1.88 (m, 6H), 1.85-1.70 (m, 4H).

Step 2

The residue was purified by prep-HPLC (column: Phenomenex luna C18 150×25 mm, 10 μm; mobile phase: [water (0.225% FA)-ACN]; B %: 1%-30%, 10 min) to give Additional Example 10 (67.0 mg, 87.6 gmol, 40.4% yield, 99.0% purity, FA) as a white solid.

LCMS (Method 1): Rt=0.799 min, MS m/z 711.3 [M+H]⁺, theoretical mass: 710.33 HPLC (method 7), Rt=2.519 min.

¹H-NMR 400 MHz (D₂O): δ ppm: 7.64 (s, 2H), 7.59-7.50 (m, 10H), 5.25-5.17 (m, 4H), 4.57 (d, J=10.4 Hz, 2H), 3.28-3.10 (m, 6H), 2.98-2.88 (m, 2H), 2.75-2.67 (m, 2H), 2.47-2.35 (m, 2H), 2.13-2.02 (m, 6H), 2.08-1.87 (m, 2H), 1.86-1.73 (m, 4H).

### Additional Example 11, Compound ID P2B-E

### 2-[[3-[[carboxy-[(3R)-quinuclidin-3-yl]methyl]carbamoyl] benzoyl]amino]-2-[(3R)-quinuclidin-3-yl]acetic acid

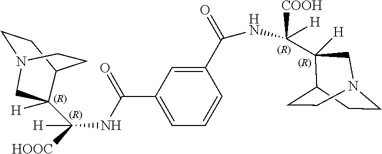

Step 1

To a solution of methyl 2-amino-2-[(3R)-quinuclidin-3-yl]acetate (100.00 mg, 504.39 gmol) in CHCl₃ (4 mL) was added benzene-1,3-dicarbonyl chloride (51.20 mg, 252.20 gmol) at 30° C. The mixture was stirred for 16 hours at 30° C. The mixture was concentrated under reduced pressure to give a crude product as a yellow solid.

LCMS: Rt=0.881 min, MS m/z 527.3 [M+H]⁺

Step 2

To a solution of the bis methyl ester (165.00 mg, 313.31 gmol) in THF (4 mL) was added LiOH (96.00 mg, 4.01 mmol, 12.79) in H₂O (4 mL) at 30° C. The mixture was stirred for 2 hours at 30° C. The mixture was concentrated under reduced pressure to remove THF. To the residue was added water (10 mL) and 1M HCl (aq) to pH=2. The mixture was concentrated under reduced pressure to give a crude product. The crude product was purified by preparative HPLC to afford Additional Example 11 (37.20 mg, 63.79 gmol, 40.72% yield, 98% purity, 2HCl) as a white solid.

¹H-NMR 400 MHz (D₂O): δ ppm: 8.10-8.01 (m, 1H), 7.88 (dd, J=1.7, 7.8 Hz, 2H), 7.54 (t, J=7.8 Hz, 1H), 4.62 (d, J=11.0 Hz, 2H), 3.59-3.50 (m, 2H), 3.38-3.15 (m, 8H), 3.11-2.99 (m, 2H), 2.71-2.54 (m, 2H), 2.26-2.07 (m, 4H), 2.06-1.78 (m, 6H).

LCMS Rt=5.9 min, MS m/z=499.3 [M+H]⁺, theoretical mass: 498

### Biological Assays

### MIRA Immunoturbidimetric Assay

The CRP immunoturbidimetric assay on the Roche COBAS MIRA Plus autoanalyser, utilises two different sized latex particles that are covalently coupled with two different monoclonal antibodies with specificity for different CRP epitopes (10). The assay was validated by Roche for measurement of native pentameric CRP, for which it has high sensitivity and specificity and a high upper detection limit; it was calibrated against a standard produced in our laboratory. Serendipitously, one of the assay's antibodies binds to an epitope present on the ligand binding B face of CRP. Thus, when the binding pocket is occupied by ligand or is occluded, for example by B face to B face complexing of pentamers, the assay fails to detect CRP although it is demonstrable by other types of assays that employ antibodies which bind to different epitopes. Bivalent compounds such as BPC8 and APL-2191 were designed to crosslink pairs of CRP pentamers. Therefore, inhibition of CRP recognition in the MIRA assay is a convenient tool to monitor the efficacy and potency of complex formation between such ligands and CRP (4).

CRP concentrations were measured in the presence and absence of ligands by the COBAS MIRA autoanalyser. Concentrated Tris-calcium buffer (×10 TC) was prepared in MilliQ water from trishydroxymethyamine (100 mM), calcium chloride (20 mM) and sodium chloride (1.4 M). The pH was adjusted to 8.0 using HCl and sodium azide was added (0.1% w/v); the buffer was stored at 4° C. A tenfold diluted working buffer (TC) was prepared by dilution 100 ml of the ×10 concentrated buffer with 900 ml of MilliQ water. Human CRP was isolated, purified and characterised as previously reported (5, 8, 14, 15) and stored frozen at −80° C. When required, stock CRP was thawed at 37° C. and working dilutions prepared that were kept at 4° C. for the duration of an experiment. CRP concentration was determined spectrophotometrically (Beckman Coulter DU 650) in quartz cuvettes with a 1 cm light path, by measuring A₂₈₀ after correction for absorbance at 320 nm (light scattering) and using the measured absorption coefficient A(1%, 1 cm)=17.5 for human CRP (7). Human CRP at ˜90 μg/ml (0.78 μM of pentamer) in TC buffer was prepared from a stock solution; a 75 μl aliquot was used in the assay. Compounds were supplied by Wuxi AppTec (Wuhan, China) as solids. They were dissolved in TC buffer at suitable concentrations, depending on solubility, of up to 10 mM (labelled S1). They were then serially diluted 1:2 with TC buffer (100 μl ligand+200 μl TC) to provide up to 9 dilutions, S2-S10. A TC buffer control (S0) was included in each assay. A 15 μl volume of each ligand solution was incubated with 75 μl of CRP for 1 h at room temperature. The final concentrations were 0.73 μM native pentameric CRP, ligands S1-S10=up to 625-0.03 μM, corresponding to ligand:CRPᵣ ratios of 850-0.04. Where compounds were of reduced solubility in TC buffer, lower stock concentrations were used (from 0.6 mM), corresponding to final top assay concentrations of 100 μM.

Data are expressed as measured CRP (mg/L) against final total ligand concentration (M) and were plotted using Sigmaplot (V14) using the 4 parameter logistic curve y=min+(max−min)/(1+(x/EC₅₀)⁻Hill slope) to calculate EC₅₀. Where appropriate, samples were also measured in whole normal human serum following the addition of a known amount of human CRP. All compounds were assayed in comparison with a highly purified preparation of bis(phosphocholine)octane (BPC8), that was prepared by Carbogen AMCIS AG and diluted into sterile water at 10 mM concentration. It was stored at −80° C. The solution was diluted into TC buffer as required.

Table 1 shows the data for the MIRA immunoturbidimetric assay for Additional Examples 1-12

TABLE 1

| Example No. | Compound ID | IC50 (μM) |
|---|---|---|
| 1 | APL-2191 | 0.60 |
| 2 | APL-6968 | 0.65 |
| 3 | APL-6969 | 1.22 |
| 4 | APL-6970 | 0.58 |
| 5 | APL-6971 | 0.73 |
| 6 | APL-6972 | 0.56 |
| 7 | APL-6973 | 0.51 |
| 8 | APL-6974 | 0.59 |
| 9 | APL-6975 | 0.55 |
| 10 | APL-6976 | 0.67 |
| 11 | P2B-E | 2.02 |
| 12 | BPC8 | 1.2 |

The Examples of formula (I) are RR,RR stereoisomers. The other stereoisomers of this structure have lesser or no activity. The SS,SS isomer is the most active alternative isomer (denoted QA,QA Quinuclidine, Amino Acid: SS,SS IC50 34.4 uM, RS,RS IC50>1000 uM, SR,SR IC50>1000 uM, RS,RR>1000 uM). Alternative isomers may be prepared by one skilled in the art according to the methods above using the desired stereoisomers with a suitable protecting group strategy employed.

Each document cited in this text (“application cited documents”) and each document cited or referenced in each of the application cited documents, and any manufacturer's specifications or instructions for any products mentioned in this text and in any document incorporated into this text, are hereby incorporated herein by reference; and, technology in each of the documents incorporated herein by reference can be used in the practice of this invention.

The present invention claims priority from United Kingdom Patent Application GB2002299.2 filed on 19 Feb. 2020, the entire content of which is also expressly incorporated herein by reference.

### REFERENCES

1. Pepys M. B. (2018) “The Pentraxins 1975-2018: Serendipity, Diagnostics and Drugs.” Front. Immunol. 9: 2382. doi: 10.3389/fimmu.2018.02382.
2. Griselli, M. Herbert, J., Hutchinson, W. L., Taylor, K. M., Sohail, M., Krausz, T. and Pepys, M. B., (1999) “C-reactive protein and complement are important mediators of tissue damage in acute myocardial infarction.” J. Exp. Med. 190: 1733-1739.
3. Gill, R., Kemp, J. A., Sabin, C. and Pepys, MB (2004) “Human C-Reactive Protein Increases Cerebral Infarct size after middle cerebral artery occlusion in adult rats.” J. Cereb. Blood Flow Metab. 24: 1214-1218.
4. Pepys, M. B., Hirschfield, G. M., Tennent, G. A., Gallimore, J. R., Kahan, M. C., Bellotti, V., Hawkins, P. N., Myers, R. M., Smith, M. D., Polara, A., Cobb, A. J. A., Ley, S. V., Aquilina, J. A., Robinson, C. V., Sharif, I., Gray, G. A., Sabin, C. A., Jenvey, M. C., Kolstoe, S. E., Thompson, D. and Wood, S. P. (2006) “Targeting C-reactive protein for the treatment of cardiovascular disease.” Nature 440: 1217-1221.
5. Pepys, M. B., Gallimore, J. R., Lloyd, J., Li, Z., Graham, D., Taylor, G. W., Ellmerich, S., Mangione, P. P., Tennent, G. A., Hutchinson, W. L., Millar, D. J., Bennett, G., More, J., Evans, D., Mistry, Y., Poole, S. and Hawkins, P. N. (2012) “Isolation and characterization of pharmaceutical grade human pentraxins, serum amyloid P component and C-reactive protein, for clinical use.” J. Immunol. Methods 384: 92-102.
6. de Beer, F. C., Baltz, M. L., Munn, E. A., Feinstein, A., Taylor, J., Bruton, C., Clamp, J. R. and Pepys, M. B. (1982) “Isolation and characterisation of C-reactive protein and serum amyloid P component in the rat.” Immunology 45: 55-70.
7. Nelson, S. R., Tennent, G. A., Sethi, D., Gower, P. E., Ballardie, F. W., Amatayakul-Chantler, S. and Pepys, M. B. (1991) “Serum amyloid P component in chronic renal failure and dialysis.” Clin. Chim. Acta 200(2-3): 191-200.
8. de Beer, F. C. and Pepys, M. B. (1982) “Isolation of human C-reactive protein and serum amyloid P component.” J. Immunol. Methods 50: 17-31.
9. Vigushin, D. M., Pepys, M. B. and Hawkins, P. N. (1993) “Metabolic and scintigraphic studies of radioiodinated human C-reactive protein in health and disease” J. Clin. Invest. 91: 1351-1357.
10. Eda, S., Kaufmann, J., Roos, W. and Pohl, S. (1998) “Development of a new microparticle-enhanced turbidometric assay for C-reactive protein with superior features in analytical sensitivity and dynamic range.” J. Clin. Lab. Anal. 12: 137-144.
11. Khuseyinova, N., Imhof, A., Trischler, G., Rothenbacher, D., Hutchinson, W. L., Pepys, M. B. and Koenig, W. (2003) “Determination of C-reactive protein: comparison of three high-sensitivity immunoassays.” Clin. Chem. 49: 1691-1695.
12. Thompson, D. et al. (1999) “The physiological structure of human C-reactive protein and its complex with phosphocholine.” Structure 7: 169-177.
13. Laurell, C.-B. (1972) “Electroimmunoassay.” Scand. J. Clin. Lab. Invest. 29(Suppl. 124): 21-37.
14. Pepys, M. B., Dash, A. C. and Ashley, J. (1977) “Isolation of C-reactive protein by affinity chromatography.” Clin. Exp. Immunol. 30: 32-37.
15. Carlucci, F., Cook, H. T., Garg, A., Pepys, M. B. and Botto, M. (2010) “Lack of effect of a single injection of human C-reactive protein on murine lupus or nephrotoxic nephritis.” Arthritis Rheum. 62: 245-249.

1. An agent for use in medicine, wherein the agent comprises a compound of Formula (I):
B-L-B′ (I)
wherein:
B and B′ are independently selected from groups of formula B-I: 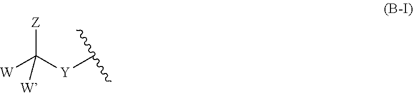
wherein:
Z is selected from —COOH, —CH₂COOH, —PO(OH)(OR¹), or —CH₂PO(OH)(OR¹),
wherein R¹ is H or a phosphate protecting group;
W is an alicyclic amine group having from 5 to 12 carbon atoms and at least one amine nitrogen atom;
W′ is H, or W′ is linked to W to form said alicyclic amine group; and
Y is selected from —NH—, —N(CH₃)—, —CH₂—, —NHCO—, —CH₂CONH—, —CONH—, —CH₂NHCO—, or —NHCH₂—; and
L is a linker group selected from: a direct bond; a saturated or unsaturated chain of from 1 to 12 carbon atoms in which from 1 to 4 of the carbon atoms are optionally replaced by O or S, and wherein the chain is optionally substituted by one or more groups selected from halogen, C1-C6 alkyl, C2-C6 alkenyl, C6-C12 (hetero)aryl, C6-C12 (hetero)arylC1-C4alkyl, or C1-C6 alkoxy; or L is a group of formula -L¹-Cy-L²- wherein Cy is a (hetero)aryl or (hetero)cycloalkyl group and L¹ and L² are independently selected from a direct bond or C1-C4 alkenyl groups in which one or two of the carbon atoms are optionally replaced by O or S, including individual stereoisomers thereof, stereoisomer mixtures thereof, and pharmaceutically acceptable salts, solvates, prodrugs or derivatives thereof.

2. An agent for use in medicine according to claim 1 wherein B and B′ are the same

3. An agent for use in medicine according to claim 2, wherein the compound of Formula (I) is a palindromic compound.

4. An agent for use in medicine according to any preceding claim, wherein the alicyclic amine group W is selected from the group consisting of: a piperidine, a pyrrolidine, a piperazine, a pyrimidine, a morpholine, or an aza or diaza bicyclic [2.2.2], [2.2.1] or [3.2.1] bicyclic group, optionally wherein the amine nitrogen is alkylated with one or more C1-C4 alkyl groups to provide a tertiary or quaternary amine group in the ring.

5. An agent for use in medicine according to claim 4, wherein the alicyclic amine group W is quinuclidin-3-yl, quinuclidin-4-yl, N-methylpyrollidone-3-yl or N-methylpiperidine-4-yl.

6. An agent for use in medicine according to any preceding claim, wherein the groups B and/or B′ are selected from the following groups B-XIV to B-XXI: 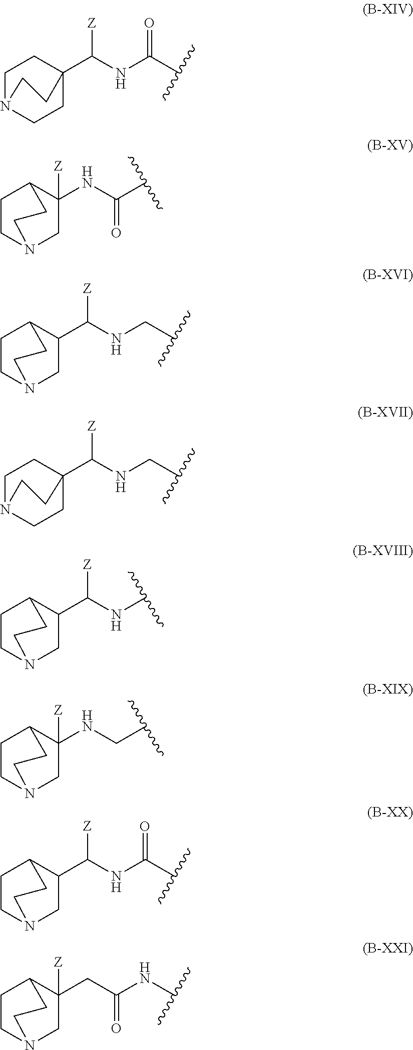
wherein Z is as defined in claim 1, preferably wherein Z is —COOH or —PO(OH)OR¹, wherein R¹ is a phosphate protecting group as defined above, suitably wherein R¹ is benzyl (C₆H₅CH₂—).

7. An agent for use in medicine according to claim 6, wherein the groups B and B′ are selected from the groups B-XVI and B-XX wherein Z is —COOH or —PO(OH)OR¹ wherein R¹ is selected from the group consisting of C1-C7 alkyl and C6-C12arylC1-C4alkyl.

8. An agent for use in medicine according to any preceding claim, wherein R¹ is selected from C1-C7 alkyl groups, C1-C7 alkenyl groups, or a C5-C6 aryl group linked to the phosphate through a C1-C4 alkylene group, any of which may optionally substituted with one or more halogen, —CN, or nitro groups, preferably a benzyl (C₆H₅CH₂—) group.

9. An agent for use in medicine according to any preceding claim, wherein the linker group L is selected from a direct bond, a saturated or unsaturated alkylene or alkenylene chain of from 1 to 8 carbon atoms wherein the chain is optionally substituted by one or more C1-C4 alkyl groups or phenyl groups, or a linker group selected from one of L-I to L-IV as follows: 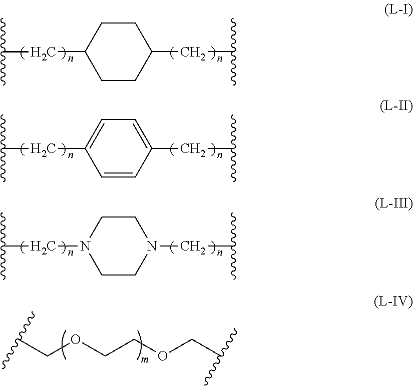
wherein n and m are 0, 1 or 2.

10. An agent for use in medicine according to any preceding claim, wherein the linker group L is selected from a direct bond, an alkylene (—CₙH₂ₙ—) or alkenylene (—CₙH₂ₙ₋₂—) chain of 2, 4, 6 or 8 carbon atoms, or a linker group from selected from one of L-V to L-VIII as follows: 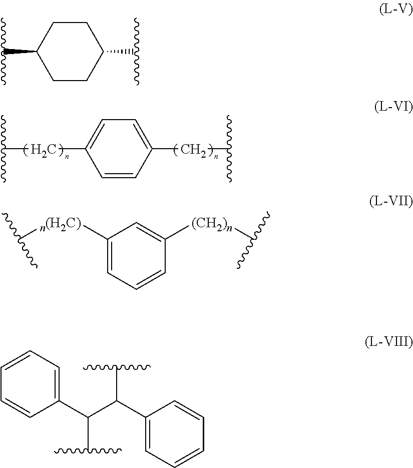
wherein n is 0 or 1.

11. An agent for use in medicine according to any preceding claim, wherein the compound of Formula (I) is has the following Formula (II): 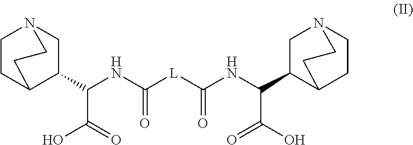
or the following Formula (III): 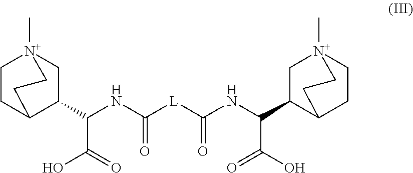
wherein L is a direct bond or a linker group of formula —(CH₂)ₙ— wherein n is from 1 to about 8, preferably wherein L is a direct bond or a linker group of formula —(CH₂)ₙ— wherein n is 2, 4, 6 or 8.

12. An agent for use in medicine according to claim 1, wherein the agent comprises a compound of Formula (IV): 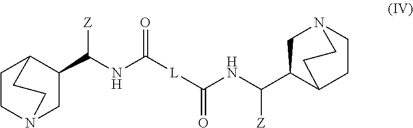
wherein:
Z are independently selected from —COOH, —CH₂COOH, —PO(OH)(OR¹), or —CH₂PO(OH)(OR¹), wherein R¹ is a phosphate protecting group; and
L is a linker group selected from:
a direct bond;
—CH₂CH₂— or —CH═CH— (preferably trans-CH═CH—), optionally substituted by one or more groups selected from halogen, hydroxy, trifluoromethyl, C1-C4 alkyl, C1-C4 alkoxy, C2-C4 alkenyl, or C6-C12 (hetero)aryl;
an aryl linker group Ar; or
a group of Formula (VI): 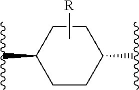
wherein R represents one, two or three optional substituents selected from halogen, hydroxy, C1-C4 (cyclo)alkyl or C1-C4 (cyclo)alkoxy having the alkyl group optionally substituted with one or more halogen atoms, or C2-C4 alkenyl
including individual stereoisomers thereof, stereoisomer mixtures thereof, and pharmaceutically acceptable salts, solvates, prodrugs or derivatives thereof.

13. An agent for use in medicine according to any of claims 1 to 10, wherein the compound of Formula (I) has the following Formula (IX): 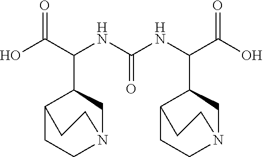 [SMILES: O=C(NC(C(=O)O)[C@H]1CN2CCC1CC2)NC(C(=O)O)[C@H]1CN2CCC1CC2]
or the following Formula (X): 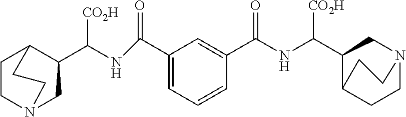 [SMILES: O=C(NC(C(=O)O)[C@H]1CN2CCC1CC2)c1cccc(C(=O)NC(C(=O)O)[C@H]2CN3CCC2CC3)c1]
or the following Formula (XI): 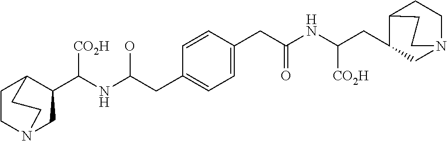 [SMILES: O=C(Cc1ccc(CC(O)NC(C(=O)O)[C@H]2CN3CCC2CC3)cc1)NC(C[C@H]1CN2CCC1CC2)C(=O)O]
or the following Formula (IV): 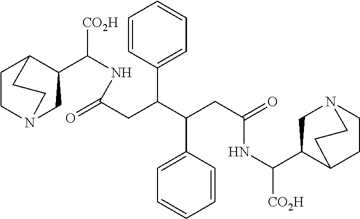 [SMILES: O=C(CC(c1ccccc1)C(CC(=O)NC(C(=O)O)[C@H]1CN2CCC1CC2)c1ccccc1)NC(C(=O)O)[C@H]1CN2CCC1CC2]
or the following Formula (XII):

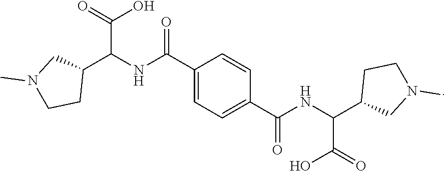

14. An agent for use in medicine according to any of claims 1 to 10, wherein the compound of Formula (I) has the following Formula (VIII): 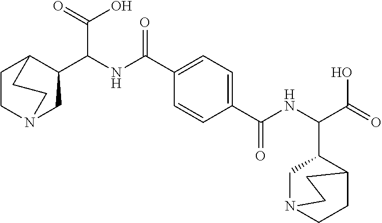 [SMILES: O=C(NC(C(=O)O)[C@H]1CN2CCC1CC2)c1ccc(C(=O)NC(C(=O)O)[C@H]2CN3CCC2CC3)cc1]
or the following Formula (XIII): 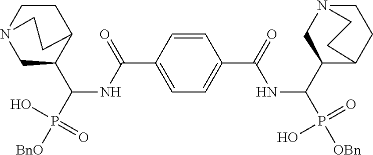
wherein Bn represents a benzyl group;
or the following Formula (XI):

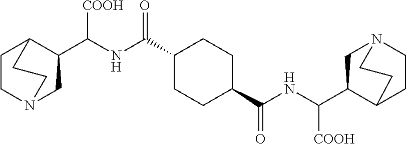 [SMILES: O=C(O)C(NC(=O)[C@H]1CC[C@H](C(=O)NC(C(=O)O)[C@H]2CN3CCC2CC3)CC1)[C@H]1CN2CCC1CC2]

15. An agent for use in medicine according to any preceding claim, wherein the compound of Formula (I) is an inhibitor of human C-reactive protein (CRP) having an IC₅₀ of about 200 μM or less as determined by the MIRA immunoturbidimetric assay as described herein, preferably about 50 μM or less, more preferably about 20 μM or less, still more preferably about 10 μM or less, or about 5 μM or less, or about 1 μM or less.

16. An agent according to any preceding claim, for use in the treatment or prevention of tissue damage in a subject having an inflammatory and/or tissue damaging condition.

17. An agent according to claim 16, wherein the inflammatory and/or tissue damaging condition comprises one or more of acute coronary syndrome, unstable angina, plaque rupture, and/or incipient atherothrombosis.

18. An agent according to claim 16, wherein the inflammatory and/or tissue damaging condition is selected from an infection, an allergic complication of infection, an inflammatory disease, ischemic or other necrosis, traumatic tissue damage and malignant neoplasia.

19. An agent according to claim 18, wherein the condition is an infection selected from a bacterial infection including sepsis, a viral infection, and a parasitic infection.

20. An agent according to claim 18, wherein the condition is an inflammatory disease selected from rheumatoid arthritis, juvenile chronic (rheumatoid) arthritis, ankylosing spondylitis, psoriatic arthritis, systemic vasculitis, polymyalgia rheumatica, Reiter's disease, Crohn's disease and familial Mediterranean fever and other autoinflammatory conditions.

21. An agent according to claim 18, wherein the condition is tissue necrosis selected from myocardial infarction, ischaemic stroke, tumour embolization and acute pancreatitis.

22. An agent according to claim 18, wherein the condition is trauma selected from elective surgery, burns, chemical injury, fractures and compression injury.

23. Use according to claim 18, wherein the condition is malignant neoplasia selected from lymphoma, Hodgkin's disease, carcinoma and sarcoma.

24. An agent according to claim 18, wherein the condition is an allergic complication of infection selected from rheumatic fever, glomerulonephritis, and erythema nodosum leprosum.

25. An agent according to claim 18, wherein the condition is an infection with a severe acute respiratory syndrome (SARS) virus, such as SARS-Cov-2.

26. A pharmaceutical composition comprising an agent according to any of claims 1 to 15 in admixture with one or more pharmaceutically acceptable excipients, diluents or carriers.
